(19) United States
(12) Patent Application Publication
(10) Pub. No.: US 2019/0240225 A1
(43) Pub. Date: Aug. 8, 2019

(54) COMBINATION OF A BCL-2 INHIBITOR AND A MCL-1 INHIBITOR, USES AND PHARMACEUTICAL COMPOSITIONS THEREOF

(71) Applicants: LES LABORATOIRES SERVIER, SURESNES CEDEX (FR); NOVARTIS AG, Basel (CH)

(72) Inventors: Andrew WEI, Surrey Hills (AU); Donia MOUJALLED, Lalor (AU); Giovanna POMILIO, Hadfield (AU); Ana Leticia MARAGNO, Croissy-sur-Seine (FR); Olivier GENESTE, Rueil-Malmaison (FR); Audrey CLAPERON, Courbevoie (FR); Heiko MAACKE, Holm (DE); Ensar HALILOVIC, Canton, MA (US); Dale PORTER, Cambridge, MA (US); Erick MORRIS, Newton, MA (US); Youzhen WANG, Newton, MA (US); Sneha SANGHAVI, Cambridge, MA (US); Prakash MISTRY, Basel (CH)

(73) Assignees: LES LABORATOIRES SERVIER, Suresnes Cedex (FR); NOVARTIS AG, Basel (FR)

(21) Appl. No.: 16/318,925

(22) PCT Filed: Jul. 21, 2017
(86) PCT No.: PCT/EP2017/068453

§ 371 Date: Jan. 18, 2019

Related U.S. Application Data
(60) Provisional application No. 62/464,554, filed on Feb. 28, 2017.
(60) Provisional application No. 62/517,252, filed on Jun. 9, 2017.

(30) Foreign Application Priority Data
Jul. 22, 2016 (EP) 16180918.1
Oct. 28, 2016 (EP) 16306420.7

(51) Int. Cl. A61K 31/519; A61K 31/4725; A61P 35/02

(52) U.S. Cl. CPC A61K 31/519; A61K 9/0019; A61P 35/02; A61K 31/4725

(57) ABSTRACT

A combination comprising a BCL-2 inhibitor and a MCL1 inhibitor, and compositions and uses thereof.

### FIELD OF THE INVENTION

The present invention relates to a combination of a BCL-2 inhibitor and a MCL1 inhibitor. The invention also relates to the use of said combination in the treatment of cancer, in particular leukaemia, lymphoma, multiple myeloma, neuroblastoma and lung cancer, and more especially acute myeloid leukaemia, T-cell acute lymphoblastic leukemia, B-cell acute lymphoblastic leukemia, mantle cell lymphoma, diffuse large B-cell lymphoma and small cell lung cancer. Also provided are pharmaceutical formulations suitable for the administration of such combinations.

### BACKGROUND OF THE INVENTION

Apoptosis is a highly regulated cell death pathway that is initiated by various cytotoxic stimuli, including oncogenic stress and chemotherapeutic agents. It has been shown that evasion of apoptosis is a hallmark of cancer and that efficacy of many chemotherapeutic agents is dependent upon the activation of the intrinsic mitochondrial pathway. Three distinct subgroups of the BCL-2 family proteins control the intrinsic apoptosis pathway: (i) the pro-apoptotic BH3 (the BCL-2 homology 3)-only proteins; (ii) the pro-survival members such as BCL-2 itself, BCL-XL, Bcl-w, MCL1 and BCL-2a1; and (iii) the pro-apoptotic effector proteins BAX and BAK (Czabotar et al, Nature Reviews Molecular cell biology 2014 Vol 15:49-63). Overexpression of the anti-apoptotic members of BCL-2 family is observed in many cancers, particularly in hematological malignancies such as mantle cell lymphoma (MCL), follicular lymphoma/diffuse large B-cell lymphoma (FL/D) and multiple myeloma (Adams and Cory Oncogene 2007 Vol 26:1324-1337). Pharmacological inhibition of the anti-apoptotic proteins BCL-2, BCL-XL and Bcl-w by the recently developed BH3-mimetics drugs such as ABT-199 and ABT-263 has emerged as a therapeutic strategy to induce apoptosis and cause tumor regression in cancer (Zhang et al, Drug Resist Updat 2007 Vol 10(6):207-17). Nevertheless, mechanisms of resistance to these drugs have been observed and investigated (Choudhary G S et al, Cell Death and Disease 2015 Vol 6, e1593; doi:10.1038/cddis.2014.525).

Acute myeloid leukaemia (AML) is a rapidly fatal blood cancer arising from clonal transformation of hematopoietic stem cells resulting in paralysis of normal bone marrow function and deaths due to complications from profound pancytopenia. AML accounts for 25% of all adult leukaemias, with the highest incidence rates occurring in the United States, Australia and Europe (WHO. GLOBOCAN 2012. Estimated cancer incidence, mortality and prevalence worldwide in 2012. International Agency for Research on Cancer). Globally, there are approximately 88,000 new cases diagnosed annually. AML continues to have the lowest survival rate of all leukaemias, with expected 5-year survival of only 24%. Although the standard therapy for AML (cytarabine in combination with anthracyclines) was conceived over 4 decades ago, the introduction of successful targeted therapies for this disease has remained an elusive goal. Furthermore, there remains a need for a chemotherapy-free treatment option for patients with AML. The concept of targeted therapy in AML has been hampered by the realisation that this disease evolves as a multi-clonal hierarchy, with rapid outgrowth of leukaemic sub-clones as a major cause of drug resistance and disease relapse (Ding L et al, Nature 2012 481:506-10). Recent clinical investigations have demonstrated the efficacy of BCL-2 inhbibitors in the treatment of AML (Konopleva M et al, American Society of Hematology 2014:118). Although these inhibitors are clinically active, it is likely that other BCL-2 family members will need to be targeted in order to enhance the overall efficacy in AML. In addition to BCL-2, MCL1 has also been identified as an important regulator of cell survival in AML (Glaser S P et al, Genes & development 2012 26:120-5).

Multiple myeloma (MM) is a rare and incurable disease that is characterized by the accumulation of clonal plasma cells in the bone marrow (BM) and accounts for 10% of all haematological malignancies. In Europe, there are approximately 27,800 new cases each year. Due to the availability of new agents in recent years including bortezomib and lenalidomide, and autologous stem cell transplant (ASCT), the survival rate has improved. However, the response to these new agents is frequently not durable and it became an evidence that new treatments are needed, especially for relapsed/refractory patients and patients with unfavorable prognostic (unfavorable cytogenetic profile). Recent investigations suggest a promising activity of BCL-2 inhibitors in a sub-group of multiple myeloma patients (Touzeau C, Dousset C, Le Gouill S, et al. Leukemia. 2014; 28(1):210-212). MCL1 has also been identified as an important regulator of cell survival in multiple myeloma (Derenne S, Monia B, Dean N M, et al. Blood. 2002; 100(1):194-199; Zhang B, Gojo I, Fenton R G. Blood. 2002; 99(6):1885-1893).

Diffuse Large B-Cell Lymphoma (DLBCL) is the most common type (25-35%) of Non-Hodgkin Lymphoma with 24 000 new patients/year. DLBCL is a heterogeneous disease with over a dozen subtypes, including double-hit/MYC translocation, Activated B-Cell (ABC) and Germinal Center B-cell (GCB). Modern immune chemotherapy (R-CHOP) cures approximately 60% of patients with DLBCL, but for the 40% remaining, there is little therapeutic option and the prognostic is poor. Thus, there is a high medical need to increase cure rates and clinical outcomes in high risk DLBCL such as ABC subtype (35% of DLBCL) that display constitutive activation of the prosurvival NF-κB pathway.

Neuroblastoma (NB) is the most common extra-cranial solid tumor in infants and children, representing 8%-10% of all childhood tumors stratified currently into low-, intermediate-, or high-risk. It accounts for approximately 15% of all cancer-related deaths in the pediatric population. The incidence of NB is 10.2 cases per million children under 15 years of age, and nearly 500 new cases are reported annually. The median age of diagnosis is 22 months. Outcomes in patients with NB have improved steadily over the last 30 years with 5-year survival rates rising from 52% to 74%. However, it is estimated that 50-60% of patients in the high-risk group experience relapse, and as such, they have only seen a modest decrease in mortality. The median time to relapse was 13.2 months, and 73% of those who relapsed were 18 months or older. Taken together, NB overall survival rates remain quite abysmal (˜20% at 5 years) despite more aggressive therapies (Colon and Chung, Adv Pediatr 2013 58:297-311). The mainstay of treatment consists of chemotherapy, surgical resection, and/or radiotherapy. However, many aggressive NB have developed resistance to chemotherapeutic agents, making the likelihood of relapse quite high (Pinto et al, J Clin Oncol 201533:3008-11). Standards of care for NB depending on risk stratification are frequently carboplatin, cisplatin cyclophosphamide, doxorubicin, etoposide, cytokines (GM-CSF and IL2), and vincristine. Relapse after initial response to chemotherapy is the major reason for treatment failure especially in high-risk NB.

Chemoresistance may derive from the activation of prosurvival BCL-2 proteins (e.g. BCL-2 and MCL1 proteins). NB express high level of BCL-2 and MCL1 and low level of BCL-XL. Inhibition of BCL-2 sensitizes cell to death and induces NB tumor regression in vivo (Ham et al, Cancer Cell 29:159-172). Antagonisms of BCL-2 and MCL1 restore chemotherapy in high-risk NB (Lestini et al, Cancer Biol Ther 2009 8:1587-1595; Tanos et al, BMC Cancer 2016 16:97). Thus, there is strong rational to combine BCL-2 and MCL1 inhibitors in naïve or resistant patients.

The present invention provides a novel combination of a BCL-2 inhibitor and a MCL1 inhibitor. The results show that with the development of potent small molecules targeting BCL-2 and MCL1, highly synergistic pro-apoptotic activity is revealed in primary human AML samples (FIG. 2A and 17) as well as in AML (FIGS. 9, 13 and 14), multiple myeloma (Example 4), lymphoma (FIGS. 4 and 12), neuroblastoma (FIG. 10), T-ALL, B-ALL cell lines (FIG. 11) and in small cell lung cancer cell lines (FIGS. 15 (a)-(e)). We also show that combined BCL-2 and MCL1 targeting in vivo is efficacious at tolerated doses in AML and lymphoma xenograft models in mouse and rats (FIGS. 2, 5, 6, 7, 8 and 16), and dramatically increases time to relapse in AML (FIGS. 2B and 2C). Furthermore, in clonogenic assays, we demonstrate that BCL-2+MCL1 targeting is specifically toxic to leukemogenic cells, but not normal hematopoietic stem cells (FIG. 3), in contrast to prior MCL1 gene targeting experiments in mice. Prior to the development of these potent and selective inhibitors, the feasibility of targeting both BCL-2 and MCL1, remained uncertain. Previous lineage-specific deletion models indicated potential risk to cardiac (Wang X et al, Genes & development. 2013; 27(12):1351-1364; Thomas R L et al, Genes & development.

2013; 27(12):1365-1377), granulocyte/hematopoietic (Opferman J et al, Science's STKE. 2005; 307(5712):1101; Dzhagalov I et al, Blood. 2007; 109(4):1620-1626; Steimer D A et al, Blood. 2009; 113(12):2805-2815), thymocyte (Dunkle A et al, Cell Death & Differentiation. 2010; 17(6):994-1002), neuronal (Arbour N et al, Journal of Neuroscience. 2008; 28(24):6068-6078) and liver function (Hikita H et al, Hepatology. 2009; 50(4):1217-1226; Vick B et al, Hepatology. 2009; 49(2):627-636) resulting from long-term ablation of MCL1. Despite these concerns, weekly, twice weekly and even daily (during 5 consecutive days) intravenous delivery of a new potent short-acting pharmacological inhibitor of MCL1 has recently been shown to be well tolerated and active against a range of cancers in vivo, including AML (Kotschy A et al, Nature. 2016; 538(7626):477-482; WO 2015/097123). The short half-life of MCL1 protein may permit sufficient time for its regeneration in critical organs, thereby permitting physiological tolerance to MCL1 inhibitors short-term exposure (Yang T et al, Journal of cellular physiology. 1996; 166(3):523-536). Until now, pulsatile inhibition of BCL-2 and MCL1 mimicking a drug-like effect has not been possible using genetically engineered approaches. The studies using BCL-2 and MCL1 inhibitors according to the present invention provide proof-of-concept demonstration that intermittent exposure to these drugs may be sufficient to trigger apoptosis and clinical response among highly sensitive diseases, such as AML, without concurrent toxicity to major organ systems.

The synergistic effect of targeting both BCL-2 and MCL1 in vitro and in vivo and the non-toxicity to normal marrow production when targeting both anti-apoptotic proteins have only been demonstrated through combination of potent small molecule inhibitors. These aspects were not anticipated by the results of gene targeting experiments, which would predict that MCL1 deletion is poorly tolerated by hematopoietic stem cells.

### SUMMARY OF THE INVENTION

The present invention relates to a combination comprising (a) a BCL-2 inhibitor of formula (I):

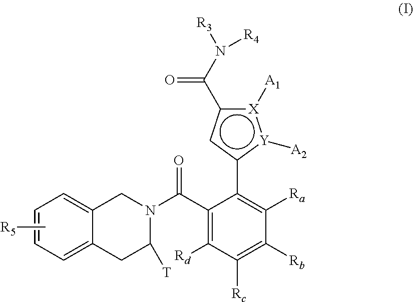

wherein:
X and Y represent a carbon atom or a nitrogen atom, it being understood that they may not simultaneously represent two carbons atoms or two nitrogen atoms,
A₁ and A₂, together with the atoms carrying them, form an optionally substituted, aromatic or non-aromatic heterocycle Het composed of 5, 6 or 7 ring members which may contain, in addition to the nitrogen represented by X or by Y, from one to 3 hetero atoms selected independently from oxygen, sulphur and nitrogen, it being understood that the nitrogen in question may be substituted by a group representing a hydrogen atom, a linear or branched (C₁-C₆)alkyl group or a group —C(O)—O-Alk wherein Alk is a linear or branched (C₁-C₆)alkyl group,
   or A₁ and A₂ independently of one another represent a hydrogen atom, a linear or branched (C₁-C₆)polyhaloalkyl, a linear or branched (C₁-C₆)alkyl group or a cycloalkyl,
T represents a hydrogen atom, a linear or branched (C₁-C₆)alkyl group optionally substituted by from one to three halogen atoms, a group (C₁-C₄)alkyl-NR₁R₂, or a group (C₁-C₄)alkyl-OR₆,
R₁ and R₂ independently of one another represent a hydrogen atom or a linear or branched (C₁-C₆)alkyl group,
   or R₁ and R₂ form with the nitrogen atom carrying them a heterocycloalkyl,
R₃ represents a linear or branched (C₁-C₆)alkyl group, a linear or branched (C₂-C₆)alkenyl group, a linear or branched (C₂-C₆)alkynyl group, a cycloalkyl group, a (C₃-C₁₀)cycloalkyl-(C₁-C₆)alkyl group wherein the alkyl moiety is linear or branched, a heterocycloalkyl group, an aryl group or a heteroaryl group, it being understood that one or more of the carbon atoms of the preceding groups, or of their possible substituents, may be deuterated,
R₄ represents an aryl group, a heteroaryl group, a cycloalkyl group or a linear or branched (C₁-C₆)alkyl group, it being understood that one or more of the carbon atoms of the preceding groups, or of their possible substituents, may be deuterated,
R₅ represents a hydrogen or halogen atom, a linear or branched (C₁-C₆)alkyl group, or a linear or branched (C₁-C₆)alkoxy group,
R₆ represents a hydrogen atom or a linear or branched (C₁-C₆)alkyl group,
Rₐ, R_{b}, R_{c} and R_{d}, each independently of the others, represent R₇, a halogen atom, a linear or branched (C₁-C₆)alkoxy group, a hydroxy group, a linear or branched (C₁-C₆)polyhaloalkyl group, a trifluoromethoxy group, —NR₇R₇′, nitro, R₇—CO—(C₀-C₆)alkyl-, R₇—CO—NH—(C₀-C₆)alkyl-, NR₇R₇′—CO—(C₀-C₆)alkyl-, NR₇R₇′—CO—(C₀-C₆)alkyl-O—, R₇—SO₂—NH—(C₀-C₆)alkyl-, R₇—NH—CO—NH—(C₀-C₆)alkyl-, R₇—O—CO—NH—(C₀-C₆)alkyl-, a heterocycloalkyl group, or the substituents of one of the pairs (Rₐ,R_{b}), (R_{b},R_{c}) or (R_{c},R_{d}) form together with the carbon atoms carrying them a ring composed of from 5 to 7 ring members, which may contain from one to 2 hetero atoms selected from oxygen and sulphur, it also being understood that one or more carbon atoms of the ring defined hereinbefore may be deuterated or substituted by from one to 3 groups selected from halogen and linear or branched (C₁-C₆)alkyl,
R₇ and R₇′ independently of one another represent a hydrogen, a linear or branched (C₁-C₆)alkyl, a linear or branched (C₂-C₆)alkenyl, a linear or branched (C₂-C₆)alkynyl, an aryl or a heteroaryl, or R₇ and R₇′ together with nitrogen atom carrying them form a heterocycle composed of from 5 to 7 ring members,
   it being understood that when the compound of formula (I) contains a hydroxy group, the latter may be optionally converted into one of the following groups: —OPO(OM)(OM′), —OPO(OM)O⁻M₁⁺), —OPO(O⁻M₁⁺)(O⁻M₂⁺), OPO(O⁻)(O⁻)M₃²⁺, OPO(OM)(O[CH₂CH₂O]ₙCH₃), or —OPO(O⁻M₁⁺)(O[CH₂CH₂O]ₙCH₃), wherein M and M′ independently of one another represent a hydrogen atom, a linear or branched (C₁-C₆)alkyl group, a linear or branched (C₂-C₆)alkenyl group, a linear or branched (C₂-C₆)alkynyl group, a cycloalkyl or a heterocycloalkyl, both composed of from 5 to 6 ring members, while M₁⁺ and M₂⁺ independently of one another represent a pharmaceutically acceptable monovalent cation, M₃²⁺ represents a pharmaceutically acceptable divalent cation, and n is an integer from 1 to 5,
   it being understood that:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens),
“cycloalkyl” means any mono- or bi-cyclic, non-aromatic, carbocyclic group containing from 3 to 10 ring members,
“heterocycloalkyl” means any mono- or bi-cyclic, non-aromatic, condensed or Spiro group composed of 3 to 10 ring members and containing from 1 to 3 hetero atoms selected from oxygen, sulphur, SO, SO₂ and nitrogen,
   it being possible for the aryl, heteroaryl, cycloalkyl and heterocycloalkyl groups so defined and the groups alkyl, alkenyl, alkynyl and alkoxy to be substituted by from 1 to 3 groups selected from: linear or branched (C₁-C₆)alkyl optionally substituted by a hydroxyl, a morpholine, 3-3-difluoropiperidine or a 3-3-difluoropyrrolidine; (C₃-C₆)spiro; linear or branched (C₁-C₆)alkoxy optionally substituted by a morpholine; (C₁-C₆)alkyl-S—; hydroxyl; oxo; N-oxide; nitro; cyano; —COOR′; —OCOR′; NR′R″; linear or branched (C₁-C₆)polyhaloalkyl; trifluoromethoxy; (C₁-C₆)alkylsulphonyl; halogen; aryl optionally substituted by one or more halogens; heteroaryl; aryloxy; arylthio; cycloalkyl; heterocycloalkyl optionally substituted by one or more halogen atoms or alkyl groups, wherein R′ and R″ independently of one another represent a hydrogen atom or a linear or branched (C₁-C₆)alkyl group optionally substituted by a methoxy,
   it being possible for the Het group defined in formula (I) to be substituted by from one to three groups selected from linear or branched (C₁-C₆)alkyl, hydroxy, linear or branched (C₁-C₆)alkoxy, NR₁′R₁″ and halogen, it being understood that R₁′ and R₁″ are as defined for the groups R′ and R″ mentioned hereinbefore,
   or its enantiomers, diastereoisomers, or addition salts thereof with a pharmaceutically acceptable acid or base,
   and (b) a MCL1 inhibitor.

Said compounds of formula (I), their synthesis, their use in the treatment of cancer and pharmaceutical formulations thereof, are described in WO 2013/110890, WO 2015/011397, WO 2015/011399 and WO 2015/011400, the contents of which are incorporated by reference.

In certain embodiments, the MCL1 inhibitor is selected from A-1210477 (Cell Death and Disease 2015 6, e1590; doi:10.1038/cddis.2014.561) and the compounds described in WO 2015/097123, WO 2016/207216, WO 2016/207217, WO 2016/207225, WO 2016/207226, or in WO 2016/033486, the contents of which are incorporated by reference.

The present invention also relates to a combination comprising (a) a BCL-2 inhibitor and (b) a MCL1 inhibitor of formula (II):

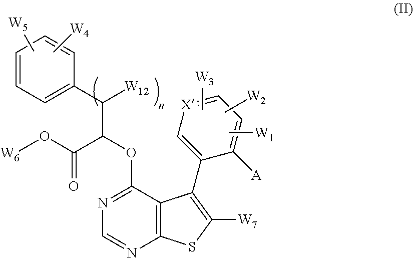

wherein:
A represents a linear or branched (C₁-C₆)alkyl group, a linear or branched (C₂-C₆)alkenyl group, a linear or branched (C₂-C₆)alkynyl group, a linear or branched (C₁-C₆)alkoxy group, —S—(C₁-C₆)alkyl group, a linear or branched (C₁-C₆)polyhaloalkyl, a hydroxy group, a cyano, —NW₁₀W₁₀′, -Cy₆ or an halogen atom,
W₁, W₂, W₃, W₄ and W₅ independently of one another represent a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)polyhaloalkyl, a hydroxy group, a linear or branched (C₁-C₆)alkoxy group, —S—(C₁-C₆)alkyl group, a cyano, a nitro group, -alkyl(C₀-C₆)—NW₈W₈′, —O-Cy₁, -alkyl(C₀-C₆)-Cy₁, -alkenyl(C₂-C₆)-Cy₁, -alkynyl(C₂-C₆)-Cy₁, —O-alkyl(C₁-C₆)—W₉, —C(O)—OW₈, —O—C(O)—W₈, —C(O)—NW₈W₈′, —NW₈—C(O)—W₈′, —NW₈—C(O)—OW₈′, -alkyl(C₁-C₆)—NW₈—C(O)—W₈′, —SO₂— NW₈W₈′, —SO₂-alkyl(C₁-C₆),
   or the substituents of one of the pairs (W₁, W₂), (W₂, W₃), (W₁, W₃), (W₄, W₅) when grafted onto two adjacent carbon atoms, form together with the carbon atoms carrying them an aromatic or non-aromatic ring composed of from 5 to 7 ring members, which may contain from one to 3 heteroatoms selected from oxygen, sulphur and nitrogen, it being understood that resulting ring may be substituted by a group selected from a linear or branched (C₁-C₆)alkyl group, —NW₁₀W₁₀′, -alkyl(C₀-C₆)-Cy₁ or an oxo,
X′ represents a carbon or a nitrogen atom,
W₆ represents a hydrogen, a linear or branched (C₁-C₈)alkyl group, an aryl, an heteroaryl group, an arylalkyl(C₁-C₆) group, an heteroarylalkyl(C₁-C₆) group,
W₇ represents a linear or branched (C₁-C₆)alkyl group, a linear or branched (C₂-C₆)alkenyl group, a linear or branched (C₂-C₆)alkynyl group, -Cy₃, -alkyl(C₁-C₆)-Cy₃, -alkenyl(C₂-C₆)-Cy₃, -alkynyl(C₂-C₆)-Cy₃, -Cy₃-Cy₄, -alkynyl(C₂-C₆)—O-Cy₃, -Cy₃-alkyl(C₀-C₆)—O-alkyl(C₀-C₆)-Cy₄, an halogen atom, a cyano, —C(O)—W₁₁, —C(O)—NW₁₁W₁₁′,
W₈ and W₈′ independently of one another represent a hydrogen atom, a linear or branched (C₁-C₆)alkyl group, or -alkyl(C₀-C₆)-Cy₁,
   or (W₈, W₈′) form together with the nitrogen atom carrying them an aromatic or non-aromatic ring composed of from 5 to 7 ring members, which may contain in addition to the nitrogen atom from one to 3 heteroatoms selected from oxygen, sulphur and nitrogen, it being understood that the nitrogen in question may be substituted by a group representing a hydrogen atom, or a linear or branched (C₁-C₆)alkyl group and it being understood that one or more of the carbon atoms of the possible substituents, may be deuterated,
W₉ represents -Cy₁, -Cy₁-alkyl(C₀-C₆)-Cy₂, -Cy₁alkyl(C₀-C₆)—O-alkyl(C₀-C₆)-Cy₂, -Cy₁-alkyl(C₀-C₆)—NW₈-alkyl(C₀-C₆)-Cy₂, -Cy₁-Cy₂-O-alkyl(C₀-C₆)-Cy₅, —C(O)—NW₈W₈′, —NW₈W₈′, —OW₈, —NW₈—C(O)—W₈′, —O-alkyl(C₁-C₆)—OW₈, —SO₂—W₅, —C(O)—OW₈, —NH—C(O)—NH—W₈,

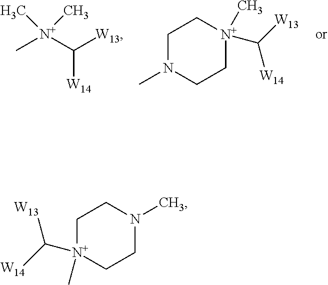

it being possible for the ammonium so defined to exist as a zwitterionic form or to have a monovalent anionic counterion,
W₁₀, W₁₀′, W₁₁ and W₁₁′ independently of one another represent a hydrogen atom or a linear or branched (C₁-C₆)alkyl group,
W₁₂ represents a hydrogen or a hydroxy group,
W₁₃ represents a hydrogen atom or a linear or branched (C₁-C₆)alkyl group,
W₁₄ represents a —O—P(O)(O⁻)(O⁻) group, a —O—P(O)(O⁻)(OW₁₆) group, a —O—P(O)(OW₁₆)(OW₁₆′) group, a —O—SO₂—O⁻ group, a —O—SO₂—OW₁₆ group, -Cy₇, a —O—C(O)—W₁₅ group, a —O—C(O)—OW₁₅ group or a —O—C(O)—NW₁₅W₁₅′ group,
W₁₅ and W₁₅′ independently of one another represent a hydrogen atom, a linear or branched (C₁-C₆)alkyl group or a linear or branched amino(C₁-C₆)alkyl group,
W₁₆ and W₁₆′ independently of one another represent a hydrogen atom, a linear or branched (C₁-C₆)alkyl group or an arylalkyl(C₁-C₆) group,
Cy₁, Cy₂, Cy₃, Cy₄, Cy₅, Cy₆ and Cy₇ independently of one another, represent a cycloalkyl group, a heterocycloalkyl group, an aryl or an heteroaryl group,
n is an integer equal to 0 or 1,
   it being understood that:
“aryl” means a phenyl, naphthyl, biphenyl, indanyl or indenyl group,
“heteroaryl” means any mono- or bi-cyclic group composed of from 5 to 10 ring members, having at least one aromatic moiety and containing from 1 to 3 heteroatoms selected from oxygen, sulphur and nitrogen,
“cycloalkyl” means any mono- or bi-cyclic non-aromatic carbocyclic group containing from 3 to 10 ring members,
“heterocycloalkyl” means any mono- or bi-cyclic non-aromatic carbocyclic group containing from 3 to 10 ring members, and containing from 1 to 3 heteroatoms selected from oxygen, sulphur and nitrogen, which may include fused, bridged or Spiro ring systems,
   it being possible for the aryl, heteroaryl, cycloalkyl and heterocycloalkyl groups so defined and the alkyl, alkenyl, alkynyl, alkoxy, to be substituted by from 1 to 4 groups selected from linear or branched (C₁-C₆)alkyl which may be substituted by a group representing a linear or branched (C₁-C₆)alkoxy which may be substituted by a linear or branched (C₁-C₆)alkoxy, a linear or branched (C₁-C₆)polyhaloalkyl, hydroxy, halogen, oxo, —NW′W″, —O—C(O)—W′, or —CO—NW′W″; linear or branched (C₂-C₆)alkenyl group; linear or branched (C₂-C₆)alkynyl group which may be substituted by a group representing a linear or branched (C₁-C₆)alkoxy; linear or branched (C₁-C₆)alkoxy which may be substituted by a group representing a linear or branched (C₁-C₆)alkoxy, a linear or branched (C₁-C₆)polyhaloalkyl, a linear or branched (C₂-C₆)alkynyl, —NW′W″, or hydroxy; (C₁-C₆)alkyl-S— which may be substituted by a group representing a linear or branched (C₁-C₆)alkoxy; hydroxy; oxo; N-oxide; nitro; cyano; —C(O)—OW′; —O—C(O)—W′; —CO—NW′W″; —NW′W″; —(C═NW′)—OW″; linear or branched (C₁-C₆)polyhaloalkyl; trifluoromethoxy; or halogen; it being understood that W′ and W″ independently of one another represent a hydrogen atom or a linear or branched (C₁-C₆)alkyl group which may be substituted by a group representing a linear or branched (C₁-C₆)alkoxy; and it being understood that one or more of the carbon atoms of the preceding possible substituents, may be deuterated,
   its enantiomers, diastereoisomers or atropisomers, or addition salts thereof with a pharmaceutically acceptable acid or base.

Said compounds of formula (II), their synthesis, their use in the treatment of cancer and pharmaceutical formulations thereof, are described in WO 2015/097123, the content of which is incorporated by reference.

In certain embodiments, the BCL-2 inhibitor is selected from the following compounds: 4-(4-{[2-(4-chlorophenyl)-4,4-dimethylcyclohex-1-en-1-yl]methyl}piperazin-1-yl)-N-[(3-nitro-4-{[(oxan-4-yl)methyl]amino}phenyl)sulfonyl]-2-[(1H-pyrrolo[2,3-b]pyridin-5-yl)oxy]benzamide (venetoclax or ABT-199); 4-(4-{[2-(4-chlorophenyl)-5,5-dimethylcyclohex-1-en-1-yl]methyl}piperazin-1-yl)-N-(4-{[(2R)-4-(morpholin-4-yl)-1-(phenylsulfanyl)butan-2-yl]amino}-3-(trifluoromethanesulfonyl)benzenesulfonyl] benzamide (navitoclax or ABT-263); oblimersen (G3139); obatoclax (GX15-070); HA14-1; (±)-gossypol (BL-193); (−)-gossypol (AT-101); apogossypol; TW-37; antimycin A, ABT-737 (Oltersdorf T et al, Nature 2005 Jun. 2; 435(7042):677-81) and compounds described in WO 2013/110890, WO 2015/011397, WO 2015/011399 and WO 2015/011400, the contents of which are incorporated by reference.

According to a first aspect of the invention, there is provided a combination comprising:

(a) a BCL-2 inhibitor of formula (I) as described herein, and
(b) a MCL1 inhibitor of formula (II) as described herein.

In another embodiment, the invention provides a combination comprising:

(a) Compound 1: N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide, or a pharmaceutically acceptable salt thereof, and
(b) a MCL1 inhibitor,
for simultaneous, sequential or separate use.

In another embodiment, the invention provides a combination comprising:

(a) Compound 4: 5-(5-chloro-2-{[3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide, or a pharmaceutically acceptable salt thereof, and
(b) a MCL1 inhibitor,
for simultaneous, sequential or separate use.

Alternatively, the invention provides a combination comprising:

(a) a BCL-2 inhibitor, and
(b) Compound 2: (2R)-2-{[(5Sₐ)-5-{3-chloro-2-methyl-4-[2-(4-methylpiperazin-1-yl)ethoxy]phenyl}-6-(5-fluorofuran-2-yl)thieno[2,3-d]pyrimidin-4-yl]oxy}-3-(2-{[1-(2,2,2-trifluoroethyl)-1H-pyrazol-5-yl]methoxy}phenyl)propanoic acid,
for simultaneous, sequential or separate use.

In another embodiment, the invention provides a combination comprising:

(a) a BCL-2 inhibitor, and
(b) Compound 3: (2R)-2-{[(5Sₐ)-5-{3-chloro-2-methyl-4-[2-(4-methylpiperazin-1-yl)ethoxy]phenyl}-6-(4-fluorophenyl)thieno[2,3-d]pyrimidin-4-yl]oxy}-3-(2-{[2-(2-methoxyphenyl)pyrimidin-4-yl]methoxy}phenyl)propanoic acid,
for simultaneous, sequential or separate use.

In another embodiment, the invention provides a combination as described herein, for use in the treatment of cancer.

In another embodiment, the invention provides the use of a combination as described herein, in the manufacture of a medicament for the treatment of cancer.

In another embodiment, the invention provides a medicament containing, separately or together,

(a) a BCL-2 inhibitor of formula (I) and
(b) a MCL1 inhibitor,
or
(a) a BCL-2 inhibitor and
(b) a MCL1 inhibitor of formula (II),
for simultaneous, sequential or separate administration, and wherein the BCL-2 inhibitor and the MCL1 inhibitor are provided in effective amounts for the treatment of cancer.

In another embodiment, the invention provides a method of treating cancer, comprising administering a jointly therapeutically effective amount of:

(a) a BCL-2 inhibitor of formula (I) and
(b) a MCL1 inhibitor,
or
(a) a BCL-2 inhibitor and
(b) a MCL1 inhibitor of formula (II),
to a subject in need thereof.

In another embodiment, the invention provides a method for sensitizing a patient who is (i) refractory to at least one chemotherapy treatment, or (ii) in relapse after treatment with chemotherapy, or both (i) and (ii), wherein the method comprises administering a jointly therapeutically effective amount of:

(a) a BCL-2 inhibitor of formula (I) and
(b) a MCL1 inhibitor,
or
(a) a BCL-2 inhibitor and
(b) a MCL1 inhibitor of formula (II),
to said patient.

In a particular embodiment, the BCL-2 inhibitor is N-(4-hydroxyphenyl)-3{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide hydrochloride (Compound 1, HCl).

In a particular embodiment, the BCL-2 inhibitor is 5-(5-chloro-2-{[3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide hydrochloride (Compound 4, HCl).

In another embodiment, the BCL-2 inhibitor is ABT-199.

In another embodiment, the MCL1 inhibitor is (2R)-2-{[(5Sₐ)-5-{3-chloro-2-methyl-4-[2-(4-methylpiperazin-1-yl)ethoxy]phenyl}-6-(5-fluorofuran-2-yl)thieno[2,3 -d]pyrimidin-4-yl]oxy}-3-(2-{[1-(2,2,2-trifluoroethyl)-1H-pyrazol-5-yl]methoxy}phenyl)propanoic acid (Compound 2).

In another embodiment, the MCL1 inhibitor is (2R)-2-{[(5Sₐ)-5-{3-chloro-2-methyl-4-[2-(4-methylpiperazin-1-yl)ethoxy]phenyl}-6-(4-fluorophenyl)thieno[2,3-d]pyrimidin-4-yl]oxy}-3-(2-{[2-(2-methoxyphenyl)pyrimidin-4-yl]methoxy}phenyl)propanoic acid (Compound 3).

### BRIEF DESCRIPTION OF THE FIGURES

FIG. 1. Expression of BCL-2 and MCL1 is prevalent in AML. 7 AML cell lines and 13 primary AML samples with >70% blasts were immunoblotted for indicated proteins, showing that BCL-2 and MCL1 proteins are dominantly expressed in contrast to BCL-XL, which was expressed in a lower proportion of samples.

FIG. 2. Combined BCL-2/MCL1 targeting has synergistic activity in AML in vitro and in vivo. (A) 54 primary AML samples were incubated with a 6-log concentration range of Compound 1 (HCl salt), Compound 2 or a 1:1 concentration in RPMI/15% FCS for 48h and the LC₅₀ determined (B) Four cohorts of NSG mice were engrafted with luciferase expressing MV4; 11 cells. Tumour engraftment was verified on day 10 (baseline) and then Compound 1, HCl 100 mg/d orally on weekdays (expressed as the free base) or Compound 2 25 mg/kg IV twice weekly administration commenced for 4 weeks. The impact of Compound 2 and the combination with Compound 1 was evidenced by reduced luciferase bulk on days 14 and 28 after starting therapy and increased overall survival (C).

FIG. 3. Toxicity assessment of combined BCL-2/MCL1 targeting on normal CD34+ cells from normal donors or leukaemic blasts. Sorted normal CD34+ or leukaemic blasts were plated and treated with Compound 1, HCl and Compound 2 at 1:1 ratio at the indicated concentrations. Combined Compound 1+Compound 2 is toxic to leukaemic but not normal CD34+ progenitors.

FIG. 4. Cell growth inhibition effect and synergy combination matrices for inhibition of cell growth (left) and Loewe excess inhibition (right) afforded by Compound 3 in combination with Compound 1, HCl in DB cells (A) and Toledo cells (B). Values in the effect matrix range from 0 (no inhibition) to 100 (total inhibition). Values in the synergy matrix represent the extent of growth inhibition in excess of the theoretical additivity calculated based on the single agent activities of Compound 3 and Compound 1, HCl at the concentrations tested. Synergistic effects occurred across a broad range of single agent concentrations.

FIG. 5. Anti-tumor effects of Compound 1, HCl, Compound 3 and the combination of Compound 1, HCl+Compound 3 in lymphoma Karpass422 xenograft model in rats.

FIG. 6. Body weight changes in animals treated with Compound 1, HCl, Compound 3 and the combination of Compound 1, HCl+Compound 3 in lymphoma Karpass422 xenograft model in rats.

FIG. 7. Anti-tumor effects of Compound 1, HCl, Compound 3 and the combination of Compound 1, HCl+Compound 3 in DLBCL Toledo xenograft model in mice.

FIG. 8. Body weight changes in animals treated with Compound 1, HCl, Compound 3 and the combination of Compound 1, HCl+Compound 3 in DLBCL Toledo xenograft model in mice.

FIG. 9. Cell growth inhibition effect and synergy combination matrices for inhibition of cell growth (left) and Loewe excess inhibition (right) afforded by Compound 3 (MCL1 inhibitor) in combination with Compound 1, HCl (BCL-2 inhibitor) in the AML cell line OCI-AML3 in two independent experiments. Values in the effect matrix range from 0 (no inhibition) to 100 (total inhibition). Values in the synergy matrix represent the extent of growth inhibition in excess of the theoretical additivity calculated based on the single agent activities of Compound 3 and Compound 1, HCl at the concentrations tested. Synergistic effects occurred across a broad range of single agent concentrations.

FIG. 10. Cell growth inhibition effect and synergy combination matrices for inhibition of cell growth (left) and Loewe excess inhibition (right) afforded by Compound 3 (MCL1 inhibitor) in combination with Compound 1, HCl (BCL-2 inhibitor) in the NB cell line LAN-6 in two independent experiments (N1: upper panel; N2: lower panel). Values in the effect matrix range from 0 (no inhibition) to 100 (total inhibition). Values in the synergy matrix represent the extent of growth inhibition in excess of the theoretical additivity calculated based on the single agent activities of Compound 3 and Compound 1, HCl at the concentrations tested.

FIG. 11. Cell growth inhibition effect and synergy combination matrices for inhibition of cell growth (left) and Loewe excess inhibition (right) afforded by Compound 3 (MCL1 inhibitor) in combination with Compound 1, HCl (BCL-2 inhibitor) in the B-ALL cell line NALM-6 in two independent experiments (N1: upper panel; N2: lower panel)

FIG. 12. Cell growth inhibition effect and synergy combination matrices for inhibition of cell growth (left) and Loewe excess inhibition (right) afforded by Com pound 3 (MCL1 inhibitor) in combination with Compound 1, HCl (BCL-2 inhibitor) in the MCL cell line Z-138.

FIG. 13. Cell growth inhibition effect and synergy combination matrices for inhibition of cell growth (left) and Loewe excess inhibition (right) afforded by Compound 3 (MCL1 inhibitor) in combination with ABT-199 (BCL-2 inhibitor) in AML cell line OCI-AML3 in two independent experiments (N1: upper panel; N2: lower panel).

FIG. 14. Exemplary cell growth inhibition effect and synergy combination matrices for inhibition of cell growth (left) and Loewe excess inhibition (right) afforded by Compound 3 (MCL1 inhibitor) in combination with Compound 4, HCl (BCL-2 inhibitor) in AML cell lines (ML-2 cells in A and OCI-AML-3 in B).

FIGS. 15 (a)-(e). Dose matrices for inhibition (left), Loewe excess inhibition (middle) and growth inhibition afforded by Compound 3 (MCL1 inhibitor) in combination with Compound 1, HCl (BCL-2 inhibitor) in a panel of SCLC cell lines.

FIGS. 16 (a)-(b). Anti-tumor effects of Compound 1, HCl, ABT-199, Compound 3 and the combination of Compound 1, HCl or ABT-199 +Compound 3 in Patient-derived primary AML model HAMLX5343 in mice.

FIG. 17. Heat-map comparison of AML sensitivity (LC₅₀) to BH3-mimetic monotherapy, or drug combinations (tested in 1:1 ratio), relative to chemotherapy (idarubicin) after 48 h exposure. Cell viability of each primary AML samples after 48 h in DMSO is shown.

### DETAILED DESCRIPTION OF THE INVENTION

The invention therefore provides in Embodiment E1, a combination comprising (a) a BCL-2 inhibitor of formula (I):

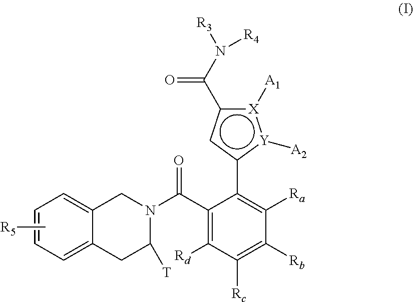

wherein:
X and Y represent a carbon atom or a nitrogen atom, it being understood that they may not simultaneously represent two carbons atoms or two nitrogen atoms,
A₁ and A₂, together with the atoms carrying them, form an optionally substituted, aromatic or non-aromatic heterocycle Het composed of 5, 6 or 7 ring members which may contain, in addition to the nitrogen represented by X or by Y, from one to 3 hetero atoms selected independently from oxygen, sulphur and nitrogen, it being understood that the nitrogen in question may be substituted by a group representing a hydrogen atom, a linear or branched (C₁-C₆)alkyl group or a group —C(O)—O-Alk wherein Alk is a linear or branched (C₁-C₆)alkyl group, or A₁ and A₂ independently of one another represent a hydrogen atom, a linear or branched (C₁-C₆)polyhaloalkyl, a linear or branched (C₁-C₆)alkyl group or a cycloalkyl,
T represents a hydrogen atom, a linear or branched (C₁-C₆)alkyl group optionally substituted by from one to three halogen atoms, a group (C₁-C₄)alkyl-NR₁R₂, or a group (C₁-C₄)alkyl-OR₆,
R₁ and R₂ independently of one another represent a hydrogen atom or a linear or branched (C₁-C₆)alkyl group,
   or R₁ and R₂ form with the nitrogen atom carrying them a heterocycloalkyl,
R₃ represents a linear or branched (C₁-C₆)alkyl group, a linear or branched (C₂-C₆)alkenyl group, a linear or branched (C₂-C₆)alkynyl group, a cycloalkyl group, a (C₃-C₁₀)cycloalkyl-(C₁-C₆)alkyl group wherein the alkyl moiety is linear or branched, a heterocycloalkyl group, an aryl group or a heteroaryl group, it being understood that one or more of the carbon atoms of the preceding groups, or of their possible substituents, may be deuterated,
R₄ represents an aryl group, a heteroaryl group, a cycloalkyl group or a linear or branched (C₁-C₆)alkyl group, it being understood that one or more of the carbon atoms of the preceding groups, or of their possible substituents, may be deuterated,
R₅ represents a hydrogen or halogen atom, a linear or branched (C₁-C₆)alkyl group, or a linear or branched (C₁-C₆)alkoxy group,
R₆ represents a hydrogen atom or a linear or branched (C₁-C₆)alkyl group,
Rₐ, R_{b}, R_{c} and R_{d}, each independently of the others, represent R₇, a halogen atom, a linear or branched (C₁-C₆)alkoxy group, a hydroxy group, a linear or branched (C₁-C₆)polyhaloalkyl group, a trifluoromethoxy group, —NR₇R₇′, nitro, R₇—CO—(C₀-C₆)alkyl-, R₇—CO—NH—(C₀-C₆)alkyl-, NR₇R₇′—CO—(C₀-C₆)alkyl-, NR₇R₇′—CO—(C₀-C₆)alkyl-O—, R₇—SO₂—NH—(C₀-C₆)alkyl-, R₇—NH—CO—NH—(C₀-C₆)alkyl-, R₇—O—CO—NH—(C₀-C₆)alkyl-, a heterocycloalkyl group, or the substituents of one of the pairs (Rₐ,R_{b}), (R_{b},R_{c}) or (R_{c},R_{d}) form together with the carbon atoms carrying them a ring composed of from 5 to 7 ring members, which may contain from one to 2 hetero atoms selected from oxygen and sulphur, it also being understood that one or more carbon atoms of the ring defined hereinbefore may be deuterated or substituted by from one to 3 groups selected from halogen and linear or branched (C₁-C₆)alkyl,
R₇ and R₇′ independently of one another represent a hydrogen, a linear or branched (C₁-C₆)alkyl, a linear or branched (C₂-C₆)alkenyl, a linear or branched (C₂-C₆)alkynyl, an aryl or a heteroaryl, or R₇ and R₇′ together with nitrogen atom carrying them form a heterocycle composed of from 5 to 7 ring members,
   it being understood that when the compound of formula (I) contains a hydroxy group, the latter may be optionally converted into one of the following groups: —OPO(OM)(OM′), —OPO(OM)(O⁻M₁⁺), —OPO(O⁻M₁⁺)(O⁻M₂⁺), —OPO(O⁻)(O⁻)M₃²⁺, —OPO(OM)(O[CH₂CH₂O]ₙCH₃), or —OPO(O⁻M₁⁺)(O[CH₂CH₂O]ₙCH₃), wherein M and M′ independently of one another represent a hydrogen atom, a linear or branched (C₁-C₆)alkyl group, a linear or branched (C₂-C₆)alkenyl group, a linear or branched (C₂-C₆)alkynyl group, a cycloalkyl or a heterocycloalkyl, both composed of from 5 to 6 ring members, while M₁⁺ and M₂⁺ independently of one another represent a pharmaceutically acceptable monovalent cation, M₃²⁺ represents a pharmaceutically acceptable divalent cation, and n is an integer from 1 to 5,
   it being understood that:
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens),
“cycloalkyl” means any mono- or bi-cyclic, non-aromatic, carbocyclic group containing from 3 to 10 ring members,
“heterocycloalkyl” means any mono- or bi-cyclic, non-aromatic, condensed or Spiro group composed of 3 to 10 ring members and containing from 1 to 3 hetero atoms selected from oxygen, sulphur, SO, SO₂ and nitrogen,
   it being possible for the aryl, heteroaryl, cycloalkyl and heterocycloalkyl groups so defined and the groups alkyl, alkenyl, alkynyl and alkoxy to be substituted by from 1 to 3 groups selected from: linear or branched (C₁-C₆)alkyl optionally substituted by a hydroxyl, a morpholine, 3-3-difluoropiperidine or a 3-3-difluoropyrrolidine; (C₃-C₆)spiro; linear or branched (C₁-C₆)alkoxy optionally substituted by a morpholine; (C₁-C₆)alkyl-S—; hydroxyl; oxo; N-oxide; nitro; cyano; —COOR′; —OCOR′; NR′R″; linear or branched (C₁-C₆)polyhaloalkyl; trifluoromethoxy; (C₁-C₆)alkylsulphonyl; halogen; aryl optionally substituted by one or more halogens; heteroaryl; aryloxy; arylthio; cycloalkyl; heterocycloalkyl optionally substituted by one or more halogen atoms or alkyl groups, wherein R′ and R″ independently of one another represent a hydrogen atom or a linear or branched (C₁-C₆)alkyl group optionally substituted by a methoxy,
   it being possible for the Het group defined in formula (I) to be substituted by from one to three groups selected from linear or branched (C₁-C₆)alkyl, hydroxy, linear or branched (C₁-C₆)alkoxy, NR₁′R₁″ and halogen, it being understood that R₁′ and R₁″ are as defined for the groups R′ and R″ mentioned hereinbefore,
   or its enantiomers, diastereoisomers, or addition salts thereof with a pharmaceutically acceptable acid or base,
   and (b) a MCL1 inhibitor,
   for simultaneous, sequential or separate use.

The invention also provides in embodiment E2 a combination comprising (a) a BCL-2 inhibitor and (b) a MCL1 inhibitor of formula (II):

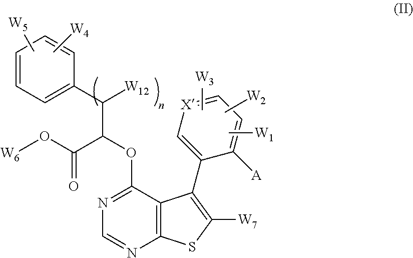

wherein:
A represents a linear or branched (C₁-C₆)alkyl group, a linear or branched (C₂-C₆)alkenyl group, a linear or branched (C₂-C₆)alkynyl group, a linear or branched (C₁-C₆)alkoxy group, —S—(C₁-C₆)alkyl group, a linear or branched (C₁-C₆)polyhaloalkyl, a hydroxy group, a cyano, -Cy₆ or an halogen atom,
W₁, W₂, W₃, W₄ and W₅ independently of one another represent a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)polyhaloalkyl, a hydroxy group, a linear or branched (C₁-C₆)alkoxy group, —S—(C₁-C₆)alkyl group, a cyano, a nitro group, -alkyl(C₀-C6)—NW₈W₈′, —O-Cy₁, -alkyl(C₀-C6)-Cy₁, -alkenyl(C₂-C₆)-Cy₁, -alkynyl(C₂-C₆)-Cy₁-C(O)—OW₈, —O—C(O)—W₈, —C(O)—NW₈W₈′, —NW₈—C(O)—W₈′, —NW₈—C(O)—OW₈′, -alkyl(C₁-C₆)—NW₈—C(O)—W₈′, —SO₂—NW₈W₈′, —SO₂-alkyl(C₁-C₆),
   or the substituents of one of the pairs (W₁, W₂), (W₂, W₃), (W₁, W₃), (W₄, W₅) when grafted onto two adjacent carbon atoms, form together with the carbon atoms carrying them an aromatic or non-aromatic ring composed of from 5 to 7 ring members, which may contain from one to 3 heteroatoms selected from oxygen, sulphur and nitrogen, it being understood that resulting ring may be substituted by a group selected from a linear or branched (C₁-C₆)alkyl group, —NW₁₀W₁₀′, -alkyl(C₀-C6)-Cy₁ or an oxo,
X′ represents a carbon or a nitrogen atom,
W₆ represents a hydrogen, a linear or branched (C₁-C₈)alkyl group, an aryl, an heteroaryl group, an arylalkyl(C₁-C₆) group, an heteroarylalkyl(C₁-C₆) group,
W₇ represents a linear or branched (C₁-C₆)alkyl group, a linear or branched (C₂-C₆)alkenyl group, a linear or branched (C₂-C₆)alkynyl group, -Cy₃, -alkyl(C₁-C₆)-Cy₃, -alkenyl(C₂-C₆)-Cy₃, -alkynyl(C₂-C₆)-Cy₃, -Cy₃-Cy₄, -alkynyl(C₂-C₆)—O-Cy₃, -Cy₃-alkyl(C₀-C₆)—O-alkyl(C₀-C₆)-Cy₄, an halogen atom, a cyano, —C(O)—W₁₁, —C(O)—NW₁₁W₁₁′,
W₈ and W₈′ independently of one another represent a hydrogen atom, a linear or branched (C₁-C₆)alkyl group, or -alkyl(C₀-C₆)-Cy₁,
   or (W₈, W₈′) form together with the nitrogen atom carrying them an aromatic or non-aromatic ring composed of from 5 to 7 ring members, which may contain in addition to the nitrogen atom from one to 3 heteroatoms selected from oxygen, sulphur and nitrogen, it being understood that the nitrogen in question may be substituted by a group representing a hydrogen atom, or a linear or branched (C₁-C₆)alkyl group and it being understood that one or more of the carbon atoms of the possible substituents, may be deuterated,
W₉ represents -Cy₁, -Cy₁-alkyl(C₀-C₆)-Cy₂, -Cy₁-alkyl(C₀-C₆)—O-alkyl(C₀-C₆)-Cy₂, -Cy₁-alkyl(C₀-C6)—NW₈-alkyl(C₀-C6)-Cy₂, -Cy₁-Cy₂-O-alkyl(C₀-C6)-Cy₅, —C(O)—NW₈W₈′, —NW₈W₈′, —OW₈, —NW₈—C(O)—W₈′, —O-alkyl(C₁-C₆)—OW₈, —SO₂—W₈, —C(O)—OW₈, —NH—C(O)—NH—W₈,

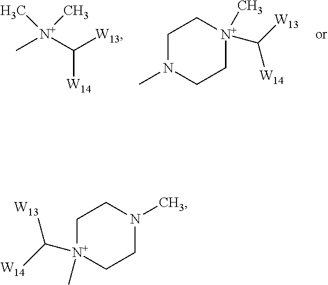

it being possible for the ammonium so defined to exist as a zwitterionic form or to have a monovalent anionic counterion,
W₁₀, W₁₀′, W₁₁ and W₁₁′ independently of one another represent a hydrogen atom or a linear or branched (C₁-C₆)alkyl group,
W₁₂ represents a hydrogen or a hydroxy group,
W₁₃ represents a hydrogen atom or a linear or branched (C₁-C₆)alkyl group,
W₁₄ represents a —O—P(O)(O⁻)(O⁻) group, a —O—P(O)(O′)(OW₁₆) group, a —O—P(O)(OW₁₆)(OW₁₆′) group, a —O—SO₂—O′ group, a —O—SO₂—OW₁₆ group, -Cy₇, a —O—C(O)—W₁₅ group, a —O—C(O)—OW₁₅ group or a —O—C(O)—NW₁₅W₁₅′ group,
W₁₅ and W₁₅′ independently of one another represent a hydrogen atom, a linear or branched (C₁-C₆)alkyl group or a linear or branched amino(C₁-C₆)alkyl group,
W₁₆ and W₁₆′ independently of one another represent a hydrogen atom, a linear or branched (C₁-C₆)alkyl group or an arylalkyl(C₁-C₆) group,
Cy₁, Cy₂, Cy₃, Cy₄, Cy₅, Cy₆ and Cy₇ independently of one another, represent a cycloalkyl group, a heterocycloalkyl group, an aryl or an heteroaryl group,
n is an integer equal to 0 or 1,
   it being understood that:
“aryl” means a phenyl, naphthyl, biphenyl, indanyl or indenyl group,
“heteroaryl” means any mono- or bi-cyclic group composed of from 5 to 10 ring members, having at least one aromatic moiety and containing from 1 to 3 heteroatoms selected from oxygen, sulphur and nitrogen,
“cycloalkyl” means any mono- or bi-cyclic non-aromatic carbocyclic group containing from 3 to 10 ring members,
“heterocycloalkyl” means any mono- or bi-cyclic non-aromatic carbocyclic group containing from 3 to 10 ring members, and containing from 1 to 3 heteroatoms selected from oxygen, sulphur and nitrogen, which may include fused, bridged or spiro ring systems,
   it being possible for the aryl, heteroaryl, cycloalkyl and heterocycloalkyl groups so defined and the alkyl, alkenyl, alkynyl, alkoxy, to be substituted by from 1 to 4 groups selected from linear or branched (C₁-C₆)alkyl which may be substituted by a group representing a linear or branched (C₁-C₆)alkoxy which may be substituted by a linear or branched (C₁-C₆)alkoxy, a linear or branched (C₁-C₆)polyhaloalkyl, hydroxy, halogen, oxo, —NW′W″, —O—C(O)—W′, or —CO—NW′W″; linear or branched (C₂-C₆)alkenyl group; linear or branched (C₂-C₆)alkynyl group which may be substituted by a group representing a linear or branched (C₁-C₆)alkoxy; linear or branched (C₁-C₆)alkoxy which may be substituted by a group representing a linear or branched (C₁-C₆)alkoxy, a linear or branched (C₁-C₆)polyhaloalkyl, a linear or branched (C₂-C₆)alkynyl, —NW′W″, or hydroxy; (C₁-C₆)alkyl-S— which may be substituted by a group representing a linear or branched (C₁-C₆)alkoxy; hydroxy; oxo; N-oxide; nitro; cyano; —C(O)—OW′; —O—C(O)—W′; —CO—NW′W″; —NW′W″; (C═NW′)—OW″; linear or branched (C₁-C₆)polyhaloalkyl; trifluoromethoxy; or halogen; it being understood that W′ and W″ independently of one another represent a hydrogen atom or a linear or branched (C₁-C₆)alkyl group which may be substituted by a group representing a linear or branched (C₁-C₆)alkoxy; and it being understood that one or more of the carbon atoms of the preceding possible substituents, may be deuterated,
   its enantiomers, diastereoisomers or atropisomers, or addition salts thereof with a pharmaceutically acceptable acid or base,
   for simultaneous, sequential or separate use.

Further enumerated embodiments (E) of the invention are described herein. It will be recognized that features specified in each embodiment may be combined with other specified features to provide further embodiments of the present invention.

E3. A combination according to E1, wherein the MCL1 inhibitor is a compound of formula (II) as defined in E2.

E4. A combination according to any of E1 to E3, wherein the BCL-2 inhibitor is N-(4-hydroxyphenyl)-3-{6-[((3S)-3-(4-morpholinylmethyl)-3,4-dihydro-2(1H)-isoquinolinyl) carbonyl]-1,3-benzodioxol-5-yl}-N-phenyl-5,6,7,8-tetrahydro-1-indolizine carboxamide.

E5. A combination according to any of E1 to E3, wherein the BCL-2 inhibitor is 5-(5-chloro-2-{[(3S)-3-(morpholin-4-ylmethyl)-3,4-dihydroisoquinolin-2(1H)-yl]carbonyl}phenyl) -N-(5 -cyano-1,2-dimethyl-1H-pyrrol-3-yl)-N-(4-hydroxyphenyl)-1,2-dimethyl-1H-pyrrole-3-carboxamide.

E6. A combination according to E4, wherein N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide is in the form of the hydrochloride salt.

E7. A combination according to E5, wherein 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide is in the form of the hydrochloride salt.

E8. A combination according to E4 or E6, wherein the dose of N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide during the combination treatment is from 50 mg to 1500 mg.

E9. A combination according to any of E1 to E8, wherein the BCL-2 inhibitor is administered once a week.

E10. A combination according to E6 or E8, wherein N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide is administered during the combination treatment once a day.

E11. A combination according to any of E1 to E3, wherein the BCL-2 inhibitor is ABT-199.

E12. A combination according to any of E1 to E11, wherein the MCL1 inhibitor is (2R)-2-{[(5Sₐ)-5-{3-chloro-2-methyl-4-[2-(4-methylpiperazin-1-yl)ethoxy]phenyl}-6-(5-fluorofuran-2-yl)thieno[2,3-d]pyrimidin-4-yl]oxy}-3-(2-{[1-(2,2,2-trifluoroethyl)-1H-pyrazol-5-yl]methoxy}phenyl)propanoic acid.

E13. A combination according to any of E1 to E11, wherein the MCL1 inhibitor is (2R)-2-{[(5Sₐ)-5-{3-chloro-2-methyl-4-[2-(4-methylpiperazin-1-yl)ethoxy]phenyl}-6-(4-fluorophenyl)thieno[2,3-d]pyrimidin-4-yl]oxy}-3-(2-{[2-(2-methoxyphenyl)pyrimidin-4-yl]methoxy}phenyl)propanoic acid.

E14. A combination according to any of E1 to E13, wherein the BCL-2 inhibitor and the MCL1 inhibitor are administered orally.

E15. A combination according to any of E1 to E13, wherein the BCL-2 inhibitor is administered orally and the MCL1 inhibitor is administered intravenously.

E16. A combination according to any of E1 to E13, wherein the BCL-2 inhibitor and the MCL1 inhibitor are administered intravenously.

E17. A combination according to any of E1 to E16, for use in the treatment of cancer.

E18. The combination for use according to E17, wherein the BCL-2 inhibitor and the MCL1 inhibitor are provided in amounts which are jointly therapeutically effective for the treatment of cancer.

E19. The combination for use according to E17, wherein the BCL-2 inhibitor and the MCL1 inhibitor are provided in amounts which are synergistically effective for the treatment of cancer.

E20. The combination for use according to E17, wherein the BCL-2 inhibitor and the MCL1 inhibitor are provided in synergistically effective amounts which enable a reduction of the dose required for each compound in the treatment of cancer, whilst providing an efficacious cancer treatment, with eventually a reduction in side effects.

E21. The combination for use according to any of E17 to E20, wherein the cancer is leukaemia.

E22. The combination for use according to E21, wherein the cancer is acute myeloid leukaemia, T-ALL or B-ALL.

E23. The combination for use according to any of E17 to E20, wherein the cancer is myelodysplastic syndrome or myeloproliferative disease.

E24. The combination for use according to any of E17 to E20, wherein the cancer is lymphoma.

E25. The combination for use according to any of E24, wherein the lymphoma is a non-Hodgkin lymphoma.

E26. The combination for use according to any of E25, wherein the non-Hodgkin lymphoma is diffuse large B-cell lymphoma or mantle-cell lymphoma.

E27. The combination for use according to any of E17 to E20, wherein the cancer is multiple myeloma.

E28. The combination for use according to any of E17 to E20, wherein the cancer is neuroblastoma.

E29. The combination for use according to any of E17 to E20, wherein the cancer is small cell lung cancer.

E30. A combination according to any of E1 to E16, further comprising one or more excipients.

E31. The use of a combination according to any of E1 to E16, in the manufacture of a medicament for the treatment of cancer.

E32. The use according to E31, wherein the cancer is leukaemia.

E33. The use according to E32, wherein the cancer is acute myeloid leukaemia, T-ALL or B-ALL.

E34. The use according to E31, wherein the cancer is myelodysplastic syndrome or myeloproliferative disease.

E35. The use according to E31, wherein the cancer is lymphoma.

E36. The use according to E35, wherein the lymphoma is a non-Hodgkin lymphoma.

E37. The use according to E36, wherein the non-Hodgkin lymphoma is diffuse large B-cell lymphoma or mantle-cell lymphoma.

E38. The use according to E31, wherein the cancer is multiple myeloma.

E39. The use according to E31, wherein the cancer is neuroblastoma.

E40. The use according to E31, wherein the cancer is small cell lung cancer.

E41. A medicament containing, separately or together,

(a) a BCL-2 inhibitor of formula (I) as defined in E1, and
(b) a MCL1 inhibitor,
for simultaneous, sequential or separate administration, and wherein the BCL-2 inhibitor and the MCL1 inhibitor are provided in effective amounts for the treatment of cancer.

E42. A medicament containing, separately or together,

(a) a BCL-2 inhibitor, and
(b) a MCL1 inhibitor of formula (II) as defined in E2,
for simultaneous, sequential or separate administration, and wherein the BCL-2 inhibitor and the MCL1 inhibitor are provided in effective amounts for the treatment of cancer.

E43. A method of treating cancer, comprising administering a jointly therapeutically effective amount of (a) a BCL-2 inhibitor of formula (I) as defined in E1, and

(b) a MCL1 inhibitor,
to a subject in need thereof.

E44. A method of treating cancer, comprising administering a jointly therapeutically effective amount of (a) a BCL-2 inhibitor, and

(b) a MCL1 inhibitor of formula (II) as defined in E2,
to a subject in need thereof.

E45. A method for sensitizing a patient who is (i) refractory to at least one chemotherapy treatment, or (ii) in relapse after treatment with chemotherapy, or both (i) and (ii), wherein the method comprises administering a jointly therapeutically effective amount of (a) a BCL-2 inhibitor of formula (I) as defined in E1, and (b) a MCL1 inhibitor, to said patient.

E46. A method for sensitizing a patient who is (i) refractory to at least one chemotherapy treatment, or (ii) in relapse after treatment with chemotherapy, or both (i) and (ii), wherein the method comprises administering a jointly therapeutically effective amount of (a) a BCL-2 inhibitor, and (b) a MCL1 inhibitor of formula (II) as defined in E2, to said patient.

“Combination” refers to either a fixed dose combination in one unit dosage form (e.g., capsule, tablet, or sachet), non-fixed dose combination, or a kit of parts for the combined administration where a compound of the present invention and one or more combination partners (e.g. another drug as explained below, also referred to as “therapeutic agent” or “co-agent”) may be administered independently at the same time or separately within time intervals, especially where these time intervals allow that the combination partners show a cooperative, e.g. synergistic effect.

The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “fixed dose combination” means that the active ingredients, e.g. a compound of formula (I) and one or more combination partners, are both administered to a patient simultaneously in the form of a single entity or dosage.

The term “non-fixed dose combination” means that the active ingredients, e.g. a compound of the present invention and one or more combination partners, are both administered to a patient as separate entities either simultaneously or sequentially, with no specific time limits, wherein such administration provides therapeutically effective levels of the two compounds in the body of the patient. The latter also applies to cocktail therapy, e.g. the administration of three or more active ingredients.

“Cancer” means a class of disease in which a group of cells display uncontrolled growth. Cancer types include haematological cancer (lymphoma and leukaemia) and solid tumors including carcinoma, sarcoma, or blastoma. In particular “cancer” refers to leukaemia, lymphoma or multiple myeloma, and more especially to acute myeloid leukaemia.

The term “jointly therapeutically effective” means that the therapeutic agents may be given separately (in a chronologically staggered manner, especially a sequence-specific manner) in such time intervals that they prefer, in the warm-blooded animal, especially human, to be treated, still show a (preferably synergistic) interaction (joint therapeutic effect). Whether this is the case can, inter alia, be determined by following the blood levels, showing that both compounds are present in the blood of the human to be treated at least during certain time intervals.

“Synergistically effective” or “synergy” means that the therapeutic effect observed following administration of two or more agents is greater than the sum of the therapeutic effects observed following the administration of each single agent.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both.

As used herein, a subject is “in need of” a treatment if such subject would benefit biologically, medically or in quality of life from such treatment.

In another aspect, provided is a method for sensitizing a human who is (i) refractory to at least one chemotherapy treatment, or (ii) in relapse after treatment with chemotherapy, or both (i) and (ii), wherein the method comprises administering a BCL-2 inhibitor of formula (I) in combination with a MCL1 inhibitor, as described herein, to the patient. A patient who is sensitized is a patient who is responsive to the treatment involving administration of a BCL-2 inhibitor of formula (I) in combination with a MCL1 inhibitor, as described herein, or who has not developed resistance to such treatment.

“Medicament” means a pharmaceutical composition, or a combination of several pharmaceutical compositions, which contains one or more active ingredients in the presence of one or more excipients.

‘AML’ means acute myeloid leukaemia.

‘T-ALL’ and ‘B-ALL’ means T-cell acute lymphoblastic leukemia and B-cell acute lymphoblastic leukemia.

‘free base’ refers to compound when not in salt form.

In the pharmaceutical compositions according to the invention, the proportion of active ingredients by weight (weight of active ingredients over the total weight of the composition) is from 5 to 50%.

Among the pharmaceutical compositions according to the invention there will be more especially used those which are suitable for administration by the oral, parenteral and especially intravenous, per- or trans-cutaneous, nasal, rectal, perlingual, ocular or respiratory route, more specifically tablets, dragées, sublingual tablets, hard gelatin capsules, glossettes, capsules, lozenges, injectable preparations, aerosols, eye or nose drops, suppositories, creams, ointments, dermal gels etc.

The pharmaceutical compositions according to the invention comprise one or more excipients or carriers selected from diluents, lubricants, binders, disintegration agents, stabilisers, preservatives, absorbents, colourants, sweeteners, flavourings etc.

By Way of Non-Limiting Example There may be Mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: magnesium aluminium silicate, starch, gelatin, tragacanth, methylcellulose, sodium carboxymethylcellulose and polyvinylpyrrolidone,
as disintegrants: agar, alginic acid and its sodium salt, effervescent mixtures.

The compounds of the combination may be administered simultaneously or sequentially. The administration route is preferably the oral route, and the corresponding pharmaceutical compositions may allow the instantaneous or delayed release of the active ingredients. The compounds of the combination may moreover be administered in the form of two separate pharmaceutical compositions, each containing one of the active ingredients, or in the form of a single pharmaceutical composition, in which the active ingredients are in admixture.

Preference is given to the pharmaceutical compositions being tablets.

### Pharmaceutical Composition of Compound 1 HCl Salt Film-Coated Tablet Containing 50 mg and 100 mg of Drug Substance

| | Amount (mg) | | |
|---|---|---|---|
| | 50 mg | 100 mg | |
| | strength | strength | Function |
| Tablet | | | |
| Compound 1 HCl salt | 52.58 | 105.16 | Drug Substance |
| equivalent in base to | 50 | 100 | |
| Lactose monohydrate | 178.51 | 357.02 | Diluent |
| Maize starch | 66.6 | 133.2 | Disintegrant |
| Povidone | 23.31 | 46.62 | Binder |
| Magnesium stearate | 3.33 | 6.66 | Lubricant |
| Silica, colloidal anhydrous | 0.67 | 1.34 | Flow agent |
| Sodium starch glycolate | 10 | 20 | Disintegrant |
| (Type A) | | | |
| For an uncoated tablet with | 335 | 670 | |
| a mass of | | | |
| Film-Coating | | | |
| Glycerol | 0.507 | 1.014 | Plasticizing agent |
| hypromellose | 8.419 | 16.838 | Film-coating agent |
| Macrogol 6000 | 0.538 | 1.076 | Smoothing agent |
| Magnesium stearate | 0.507 | 1.014 | Lubricant |
| Titanium dioxide | 1.621 | 3.242 | Pigment |
| Intermediary Vehicule | | | |
| Water, purified | qs. | qs. | Solvent |
| For a film-coated tablet with | 346.6 | 693.2 | |
| a mass of | | | |

### Pharmacological Data

### Material and Method for Examples 1-3

Primary AML patient samples: Bone marrow or peripheral blood samples from patients with AML were collected after informed consent in accordance with guidelines approved by The Alfred Hospital Human research ethics committees. Mononuclear cells were isolated by Ficoll-Paque (GE Healthcare, VIC, Aus) density-gradient centrifugation, followed by red cell depletion in ammonium chloride (NH₄Cl) lysis buffer at 37° C. for 10 minutes. Cells were then re-suspended in phosphate-buffered saline containing 2% Fetal Bovine serum (Sigma, NSW, Aus). Mononuclear cells were then suspended in RPMI-1640 (GIBCO VIC, Aus) medium containing penicillin and streptomycin (GIBCO) and heat inactivated fetal bovine serum 15% (Sigma).

Cell lines, cell culture and generating luciferase reporter cell lines: Cell lines MV4; 11, OCI-AML3, HL-60, HEL, K562, KG-1 and EOL-1 were maintained at 37° C., 5% CO₂ in RPMI-1640 (GIBCO) supplemented with 10% (v/v) fetal bovine serum (Sigma) and penicillin and streptomycin (GIBCO). MV4; 11 luciferase cell lines were generated by lentivral transductions.

Antibodies: Primary antibodies used for western blot analysis were MCL1, BCL-2, Bax, Bak, Bim, BCL-XL (generated in-house WEHI) and tubulin (T-9026,Sigma).

Cell Viability: Freshly purified mononuclear cells from AML patient samples were adjusted to a concentration of 2.5×10⁵/ml and 100 μL of cells aliquoted per well into 96 well plates (Sigma). Cells were then treated with Compound 1, HCl, Compound 2, ABT-199 (Active Biochem, NJ, USA) or idarubicin (Sigma), over a 6 log concentration range from 1 nM to 10 μM for 48 hr. For combinations assays, drugs were added at a 1:1 ratio from 1 nM to 10 μM and incubated at 37° C. 5% CO₂. Cells were then stained with sytox blue nucleic acid stain (Invitrogen, VIC, Aus) and fluorescence measured by flow cytometric analysis using the LSR-II Fortessa (Becton Dickinson, NSW, Aus). FACSDiva software was used for data collection, and FlowJo software for analysis. Blast cells were gated using forward and side scatter properties. Viable cells excluding sytox blue were determined at 6 concentrations for each drug and the 50% lethal concentration (LC₅₀, in KM) determined.

LC₅₀ determination and synergy: Graphpad Prism was used to calculate the LC₅₀ using non-linear regression. Synergy was determined by calculating the Combination Index (CI) based on the Chou Talalay method as described (Chou Cancer Res; 70(2) Jan. 15, 2010).

Colony assays: Colony forming assays were performed on freshly purified and frozen mononuclear fractions from AML patients. Primary cells were cultured in duplicate in 35 mm dishes (Griener-bio, Germany) at 1×10⁴ to 1×10⁵. Cells were plated in 0.6% agar (Difco NSW, Aus): AIMDM 2× (IMDM powder-Invitrogen), supplemented with NaHCO₃, dextran, Pen/Strep, B mercaptoethanol and asparagine):Fetal Bovine Serum (Sigma) at a 2:1:1 ratio. For optimal growth conditions all plates contained GM-CSF (10Ong per plate), IL-3(100ng/plate R&D Systems, USA) SCF (100 ng/plate R&D Systems) and EPO (4U/plate) (Growth was for 2-3 weeks in the presence and absence of drug at 37° C. at 5% CO₂ in a high humidity incubator. After incubation plates were fixed with 2.5% glutaraldehyde in saline and scored using the GelCount from Oxford Optronix (Abingdon, United Kingdom).

Western Blotting: Lysates were prepared in NP40 lysis buffer (10 mM Tris-HCl pH 7.4, 137 mM NaCl, 10% glycerol, 1% NP40), supplemented with protease inhibitor cocktail (Roche, Dee Why, NSW, Australia). Protein samples were boiled in reducing loading dye before separation on 4-12% Bis-Tris polyacrylamide gels (Invitrogen, Mulgrave, VIC, Australia), and transferred to Hybond C nitrocellulose membrane (GE, Rydalmere, NSW, Australia) for incubation with specified antibodies. All membrane-blocking steps and antibody dilutions were performed using 5% (v/v) skim milk in PBS containing 0.1% (v/v) Tween-20 phosphate-buffered saline (PBST) or Tris-buffered-saline, and washing steps performed with PBST or TB ST. Western blots were visualized by enhanced chemiluminescence (GE).

In vivo experimentation AML engraftment: Animal studies were performed under the institutional guidelines approved by the Alfred Medical Research and Education Precinct Animal Ethics Committee, MV4; 11 cells transduced with the luciferase reporter (pLUC2) were intravenously injected at 1×10⁵ cells into irradiated (100 Rad) non-obese diabetic/severe combined immunodeficient (NOD/SCID/IL2rγnull) mice as previously described (Jin et al., Cell Stem Cell 2 Jul. 2009, Volume 5, Issue 1, Pages 31-42). Engraftment was measured at day 7 by quantifying the percentage of hCD45+ cells in the PB by flow cytometry and by IVIS imaging of bioluminescent MV4; 11 cells. At day 10, mice received daily oral gavage of Compound 1, HCl (200 μL 100mg/kg—dosage expressed as the free base) dissolved in PEG400 (Sigma), absolute ethanol (Sigma) and distilled H₂0 40:10:60 or Compound 2 (200 μL 25 mg/kg) twice weekly dissolved in 50% 2-hydroxypropyl)-β-cyclodextrin (Sigma) and 50% 50mM HCl or the drug combination or vehicle, over 4 weeks. Blood counts were determined using a hematology analyzer (BioRad, Gladesville, NSW).

IVIS imaging: Bioluminescent imaging was performed using the Caliper IVIS Lumina III

XR imaging system. Mice were anaesthetised with isofleurine and injected intraperitoneally with 100 μL of 125 mg/kg luciferin (Perkin Elmer, Springvale, VIC).

### Material and Method for Example 4

Cell lines: Human myeloma cell lines (HMCLs) were derived from primary myeloma cells cultured in RPMI 1640 medium supplemented with 5% fetal calf serum from and 3 ng/mL recombinant IL-6 for IL-6 dependent cell lines. HMCLs are representative of phenotypic and genomic heterogeneity and the variability in patient's response to therapy.

MTT assay: Cell viability is measured using MTT (3-(4,5-dimethylthiazol-2-yl)-2,5-diphenyltetrazolium bromide) colorimetric survival assay. Cells are incubated with compounds in 96-well plates containing a final volume of 100 μl/well time. (2R)-2-{[(5Sₐ)-5-{3-chloro-2-methyl-4-[2-(4-methylpiperazin-1-yl)ethoxy]phenyl}-6-(5-fluorofuran-2-yl)thieno[2,3-d]pyrimidin-4-yl]oxy}-3-(2-{[1-(2,2,2-trifluoroethyl)-1H-pyrazol-5-yl]methoxy}phenyl)propanoic acid (Compound 2) is used at 9 different concentrations accordingly to single agent sensitivity. N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide hydrochloride (Compound 1, HCl) is used at a fixed dose—1 μM. At the end of each treatment, cells are incubated with 1 mg/mL MTT (50 μl MTT solution 2.5 mg/ml for each well) at 37° C. for 3 hours allowing the MTT to be metabolized. Lysis buffer (100 μl Lysis buffer: DMF (2:3)/SDS (1:3)) is added into each well to dissolve formazan cristals and after 18 h of incubation, absorbance in viable cells is measured at 570 nm using a spectrophotometer.

As control, cells are incubated with medium alone and with medium containing 0.1% DMSO. As myeloma cell growth control, myeloma cell absorbance is recorded every day (D0, D1, D2, D3 and D4).

All experiments are repeated 3 times, and each experimental condition is repeated at least in triplicate wells in each experiment.

The inhibition effect is calculated with the following formula:

Inhibition effect (%)=(1−Absorbance value of treated cells/Absorbance value of control cells)*100

### EXAMPLE 1: BCL-2 and MCL1 ARE THE DOMINANT PRO-SURVIVAL PROTEINS EXPRESSED IN AML

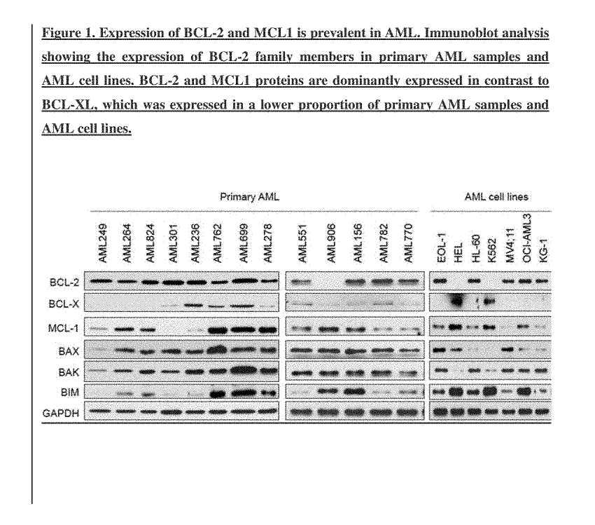

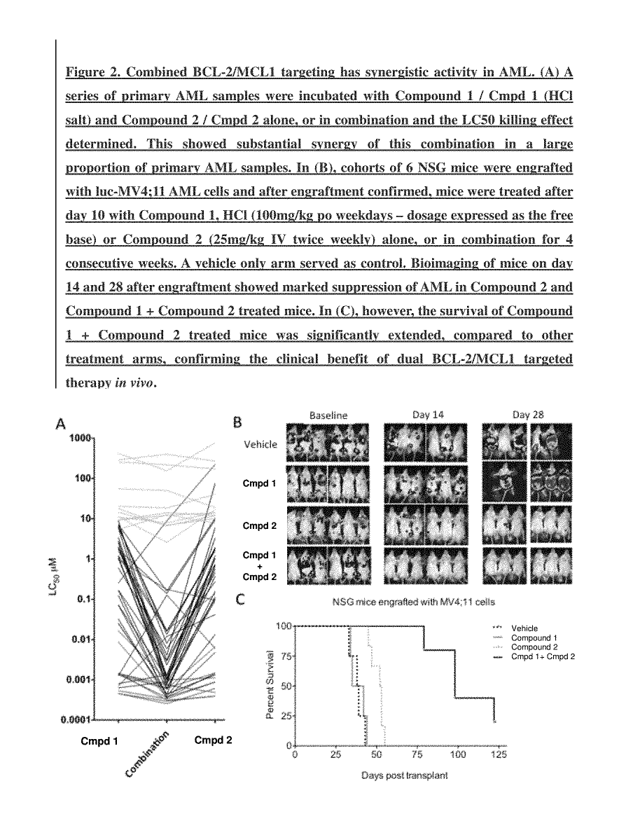

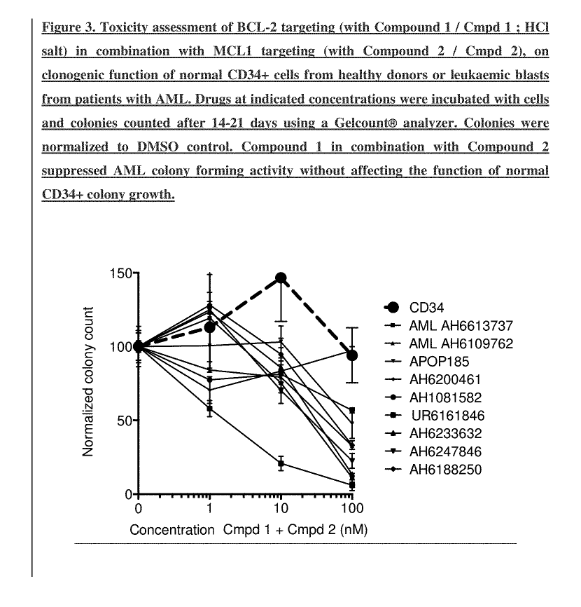

7 AML cell lines and 13 primary AML samples with >70% blasts were immunoblotted for proteins indicated in FIG. 1.

As illustrated in FIG. 1, a proteomic survey of the expression of BCL-2 family members in AML showed that, in addition to BCL-2, most primary AML samples and AML cell lines co-expressed the pro-survival protein MCL1. BCL-XL is less frequently expressed in AML.

### EXAMPLE 2: COMBINED BCL-2 and MCL1 TARGETING DISPLAYS SYNERGISTIC KILLING IN AML

54 AML patient samples were incubated with a 6-log concentration range of Compound 1 (HCl salt), Compound 2 or a 1:1 concentration in RPMI/15% FCS for 48 h and the LC₅₀ determined (FIG. 2A).

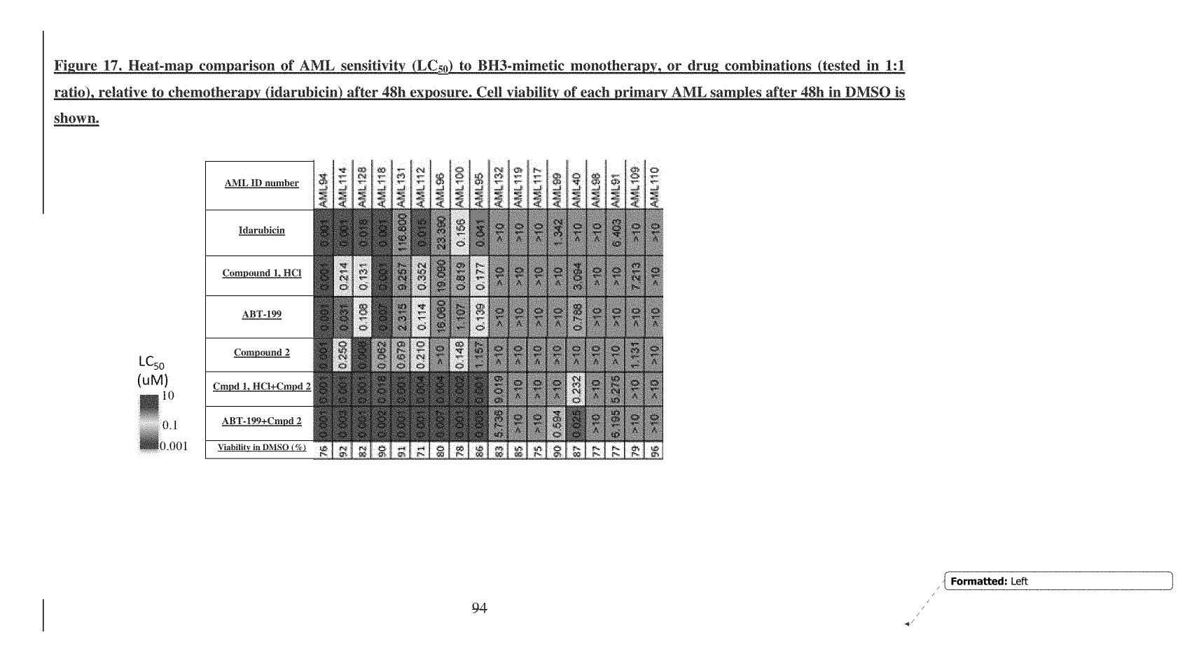

Approximately 20% of primary AML samples were highly sensitive to either Compound 1 or Compound 2, with the lethal concentration of drug required to kill 50% of primary AML blasts after 48 hours (LC₅₀) in the low nanomolar range (LC₅₀<10 nM) (FIG. 2A). In contrast, when Compound 1 and Compound 2 were combined, the proportion of AML samples that were sensitive increased dramatically to 70%, indicating synergistic activity when BCL-2 and MCL1 were simultaneously targeted (FIG. 2A). Some results are displayed in FIG. 17.

To verify the in vivo activity of this approach, luciferase expressing MV4; 11 AML cells were engrafted into NSG mice and treated with Compound 1 (HCl salt) or Compound 2 alone, or in combination and tumour burden assessed after 14 and 21 days of therapy (FIG. 2B). At the completion of 28 days of therapy, mice were followed for survival (FIG. 2C). These experiments showed that the combination of Compound 1 and Compound 2 was highly effective in vivo, validating the impressive activity observed using primary AML cells in vitro.

The data presented in FIGS. 2A-2C herein show the synergistic combination activity between Compound 1, HCl and Compound 2 in AML.

### EXAMPLE 3: COMBINED BCL-2 and MCL1 INHIBITION TARGETS LEUKAEMIC, BUT NOT NORMAL PROGENITOR FUNCTION

To assess the toxicity of BCL-2 inhibition combined with MCL1 inhibition on normal human CD34+ cells or ficolled blasts from patients with AML, clonogenic potential was assessed after 2 weeks exposure to combined therapies. Colonies were grown in agar supplemented with 10% FCS, IL3, SCF, GM-CSF and EPO over 14 days and colonies enumerated with an automated Gelcount® analyser. Assays for primary AML samples were performed in duplicate and averaged. Errors for CD34+ represent mean +/−SD of 2 independent normal donor samples. Results were normalised to the number of colonies counted in DMSO control. Indicated drug concentrations were plated on D1. Notably, Compound 1+Compound 2 suppressed AML colony forming activity without affecting the function of normal CD34+ colony growth.

Taken altogether, Examples 2 and 3 show that dual pharmacological inhibition of BCL-2 and MCL1 is a novel approach to treating AML without need for additional chemotherapy and with an acceptable therapeutic safety window.

### EXAMPLE 4: IN VITRO EVALUATION OF MULTIPLE MYELOMA CELL SURVIVAL IN RESPONSE TO A MCL1 INHIBITOR AS A SINGLE AGENT OR IN COMBINATION WITH A BCL-2 INHIBITOR

The sensitivity of 27 human multiple myeloma cell lines to Compound 1, Compound 2 or to Compound 2 in the presence of 1 μM of Compound 1 was analyzed by using MTT cell viability assay. 50% inhibitory concentrations (IC₅₀, in nM) were determined.

The results are displayed in the following table:

| | | | IC₅₀ of Compound 2 in |
|---|---|---|---|
| | Compound 1, | | the presence of 1 μM |
| Cell | HCl | Compound 2 | of Compound 1, HCl |
| lines | (IC₅₀ nM) | (IC₅₀ nM) | (nM) |
| AMO1 | 8610.3 | 0.5 | 0.2 |
| ANBL6 | 1905.0 | 79.5 | 20.8 |
| BCN | 22217.0 | 1111.4 | 59.3 |
| JIM3 | >30000 | 56.3 | 25.9 |
| JJN3 | 2692.0 | 15.6 | 2.4 |
| KMM1 | 23926.3 | 57.8 | 8.6 |
| KMS11 | 10486.7 | 44.1 | 3.9 |
| KMS12BM | 1393.7 | 44.1 | 0.03 |
| L363 | 7581.3 | 7.6 | 3.4 |
| LP1 | 9770.0 | 158.2 | 2.9 |
| MM1S | 21407.0 | 138.5 | 23.0 |
| NAN1 | 6659.0 | 5.7 | 1.4 |
| NAN3 | 8241.3 | 1.5 | 1.2 |
| NAN6 | 4074.8 | 7.1 | 1.8 |
| NAN8 | 9096.3 | 75.4 | 32.5 |
| NAN9 | 23157.6 | 9.7 | 1.1 |
| NCI-H929 | 15688.3 | 2.3 | 1.0 |
| OPM2 | 6460.7 | 9.4 | 1.2 |
| RPMI8226 | 3204.0 | 27.4 | 3.0 |
| SBN | 21273.7 | 221.1 | 14.6 |
| U266 | >30000 | 170.1 | 14.9 |
| XG1 | 9779.7 | 5.9 | 0.2 |
| XG11 | 7912.0 | 374.7 | 8.3 |
| XG2 | 15297.7 | 6.4 | 2.7 |
| XG3 | 7224.7 | 6.1 | 1.3 |
| XG6 | 8544.3 | 19.2 | 0.5 |
| XG7 | 18121.7 | 16.3 | 8.0 |

Strong synergistic activity was demonstrated when combining Compound 1 and Compound 2 in the majority of the cell lines as compared to the compounds alone.

### EXAMPLE 5: IN VITRO EFFECT ON PROLIFERATION OF COMBINING A MCL1 INHIBITOR WITH A BCL-2 INHIBITOR IN A PANEL OF 17 DIFFUSE LARGE B-CELL LYMPHOMA (DLBCL) CELL LINES

### Material and Method

Cell lines were sourced and maintained in the basic media supplemented with FCS (Fetal Calf Serum) as indicated in Table 1. In addition, all media contained penicillin (100 IU/ml), streptomycin (100 μg/ml) and L-glutamine (2 mM). Unless otherwise mentioned, culture media and supplements were from Amimed/Bioconcept (Allschwil, Switzerland).

Cell lines were cultured at 37° C. in a humidified atmosphere containing 5% CO₂ and expanded in T-75 flasks. In all cases cells were thawed from frozen stocks, expanded through ≥1 passage using appropriate dilutions, counted and assessed for viability using a CASY cell counter (Omni Life Science, Bremen, Germany) prior to plating 25 ul/well at the densities indicated in Table 1 into 384-well plates (Corning). All cell lines were determined to be free of mycoplasma contamination by PCR assay performed at Idexx Radii (Columbia, Mo., USA) and misidentification ruled out by assessment of a panel of 48 Small Nucleotide Polymorphisms (SNPs) at Asuragen (Austin, Tex., USA) or in-house.

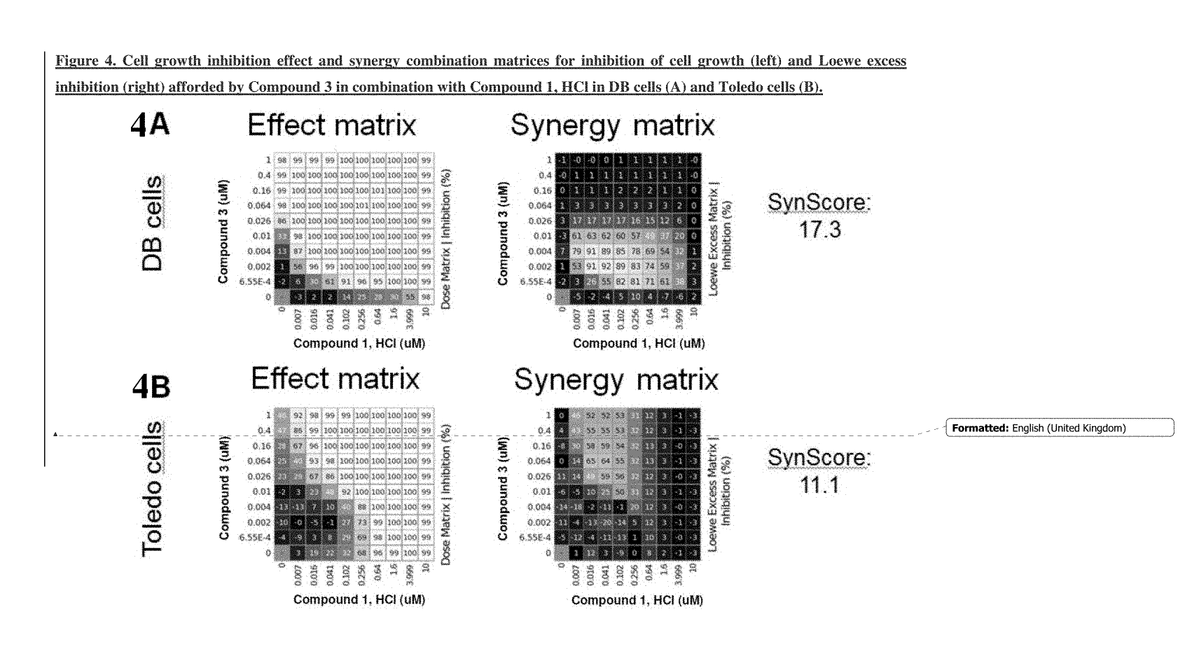

Stock solutions of compounds were prepared at a concentration of 10 mM in DMSO (Sigma) and stored at −20° C. Where necessary to afford a full dose-response curve, the stock solutions were pre-diluted in DMSO to 1′000-fold the desired start concentration (see Table 2). On the day after cell seeding, eight 2.5-fold serial dilutions of each compound were dispensed, either individually or in all possible permutations in a checkerboard fashion, directly into the cell assay plates using a non-contact 300D Digital Dispenser (ILCAN, Mannedorf, Switzerland) as outlined in FIG. 4. The final concentration of DMSO was 0.2% in all wells.

Effects of the single agents as well as their checkerboard combinations on cell viability were assessed after 2 days of incubation at 37° C./5% CO₂ by quantification of cellular ATP levels using CellTiterGlo (Promega, Madison, Wis., USA) at 25 μL reagent/well and n=2 replicate plates per condition. Luminescence was quantified on a M1000 multipurpose platereader (ILCAN, Mannedorf, Switzerland). The number/viability of cells at time of compound addition was likewise assessed and used to assess the degree of the population doubling time of a particular cell line.

Single agent IC₅₀s were calculated using standard four-parametric curve fitting. Potential synergistic interactions between compound combinations were assessed using the Excess Inhibition 2D matrix according to the Loewe additivity model and are reported as Synergy Score (Lehar et al, Nat Biotechnol. 2009 July; 27(7): 659-666). All calculations were performed using the Combination Analysis Module in-house software. IC₅₀ are defined as the compound concentration at which the CTG signal is reduced to 50% of that measured for the vehicle (DMSO) control.

The interpretation of the Synergy Score is as follows:

### SS˜0→Additive

### SS>1→Weak Synergy

### SS>2→Synergy

TABLE 1
Identity and assay conditions for the 17 Diffuse Large B-Cell
Lymphoma cell lines used in the combination experiments.

| Cell | Medium | | Doubling | Cell number |
|---|---|---|---|---|
| line | (source) | % FCS | time (hours) | seeded/well |
| DB | RPMI (ATCC) | 10 | 31.7 | 500 |
| DOHH-2 | RPMI (DSMZ) | 10 | 25.3 | 500 |
| HT | RPMI (ATCC) | 10 | 34.3 | 2000 |
| JM1 | Iscove's | 10 | 22.8 | 500 |
| | MEM*(ATCC) | | | |
| KARPAS-422 | RPMI (DSMZ) | 10 | 26.5 | 500 |
| NU-DHL-1 | RPMI (DSMZ) | 20 | 28.0 | 500 |
| OCI-LY-19 | MEM alpha | 20 | 25.8 | 500 |
| | (DSMZ) | | | |
| Pfeiffer | RPMI (ATCC) | 10 | 46.2 | 2000 |
| RL | RPMI (ATCC) | 10 | 28.9 | 500 |
| SU-DHL-10 | RPMI (DSMZ) | 20 | 105.7 | 1000 |
| SU-DHL-4 | RPMI (DSMZ) | 10 | 25.2 | 500 |
| SU-DHL-5 | RPMI (DSMZ) | 20 | 25.9 | 500 |
| SU-DHL-6 | RPMI (DSMZ) | 20 | 30.1 | 500 |
| SU-DHL-8 | RPMI (DSMZ) | 20 | 23.6 | 500 |
| Toledo | RPMI (ATCC) | 10 | 49.6 | 2000 |
| U-937 | RPMI (ATCC) | 10 | 28.7 | 500 |
| WSU-DLCL2 | RPMI (DSMZ) | 10 | 26.1 | 500 |

*This medium was further complemented with 50 μM 2-mercaptoethanol. Doubling times were calculated based on the difference in ATP levels at the end compared to the beginning of compound incubation.

TABLE 2
Single agent IC₅₀ values for Compound 3 and Compound 1, HCl, as well as the
synergy scores for their combination are indicated. Interactions were deemed
synergistic when scores ≥ 2.0 where observed.

| | Compound 3 | | | Compound 1, HCl | | | Combination | |
|---|---|---|---|---|---|---|---|---|
| | Start | Abs | Max | Start | Abs | | | Synergy |
| | conc | IC₅₀ | Inh | conc | IC₅₀ | Max Inh | Synergy | Score |
| Cell Line | [uM] | [uM] | [%] | [uM] | [uM] | [%] | Score | Error |
| DB | 1 | 0.0129 | 99.2 | 10 | 2.76 | 95.7 | 17.3 | 0.18 |
| DOHH-2 | 0.1 | 0.00122 | 98.3 | 10 | 0.156 | 101.9 | 2.90 | 0.11 |
| HT | 1 | 0.00638 | 99.3 | 10 | ≥10 | 37.2 | 2.35 | 0.06 |
| JM1 | 1 | 0.0588 | 99.7 | 10 | 0.697 | 99.1 | 5.83 | 0.25 |
| KARPAS- | 1 | 0.00214 | 98.3 | 10 | 2.18 | 90.9 | 9.74 | 0.32 |
| 422 | | | | | | | | |
| NU-DHL-1 | 1 | 0.0579 | 98.1 | 10 | 0.0515 | 102.1 | 4.97 | 0.12 |
| OCI-LY-19 | 1 | 0.0604 | 98.5 | 10 | 0.0895 | 100.1 | 3.92 | 0.07 |
| Pfeiffer | 1 | 0.0426 | 82.7 | 10 | 4.50 | 92.0 | 3.44 | 0.19 |
| RL | 10 | 3.03 | 95.4 | 10 | 0.281 | 99.0 | 12.7 | 0.38 |
| SU-DHL- | 1 | 0.00384 | 98.3 | 10 | ≥10 | 43.9 | 2.44 | 0.26 |
| 10 | | | | | | | | |
| SU-DHL-4 | 1 | 0.0178 | 99.5 | 10 | 0.86 | 96.9 | 10.8 | 0.28 |
| SU-DHL-5 | 0.1 | 0.00094 | 98.2 | 10 | ≥10 | 49.6 | 1.45 | 0.14 |
| SU-DHL-6 | 1 | 0.00213 | 99.1 | 10 | 0.614 | 101.0 | 4.57 | 0.21 |
| SU-DHL-8 | 10 | 0.305 | 95.6 | 10 | ≥10 | 28.7 | 9.88 | 0.20 |
| Toledo | 1 | ≥1 | 45.8 | 10 | 0.137 | 101.9 | 11.1 | 0.51 |
| U-937 | 1 | 0.00832 | 97.1 | 10 | 6.83 | 62.1 | 5.63 | 0.20 |
| WSU- | 1 | 0.00792 | 99.0 | 10 | 1.02 | 99.3 | 8.67 | 0.13 |
| DLCL2 | | | | | | | | |

“Start conc” means start concentration.
“Abs IC₅₀” means absolute IC₅₀.
“Max Inh” means maximum inhibition.

### Results

The effect on proliferation of combining the MCL1 inhibitor Compound 3 with the BCL-2 inhibitor Compound 1, HCl was assessed in a panel of 17 Diffuse Large B-Cell Lymphoma (DLBCL) cell lines.

Compound 3 as single agent strongly inhibited the growth of the majority of the 17 DLBCL lines tested (Table 1). Thus, 14 cell lines displayed IC₅₀s below 100 nM, and an additional 1 cell lines displayed IC₅₀s between 100 nM and 1 uM. Only 2 cell lines displayed an IC₅₀ above 1 uM.

Compound 1, HCl as single agent also inhibited the growth of the majority of the 17 DLBCL lines tested, although slightly less potent (Table 2). Thus, 2 cell lines displayed IC₅₀s below 100 nM, and 6 cell lines displayed IC₅₀s between 100 nM and 1 uM. Nine cell line displayed an IC₅₀ above 1 uM (four of which above 10 uM).

In combination, Compound 3 and Compound 1, HCl treatment caused synergistic growth inhibition (i.e. Synergy Scores above 2—Lehar et al, Nat Biotechnol. 2009 July; 27(7): 659-666) in 16 out of 17 DLBCL cell lines tested (Table 2). In 5 cell lines, the synergy effect was marked, with synergy scores between 5 and 10. In 4 cell lines, the synergy effect was exceptional, achieving synergy scores between 10 and 17.3. Importantly, the synergy was not dependent on single agent anti-proliferative effects, and in fact was particularly strong at concentrations of Compound 3 and Compound 1 that did not display an anti-proliferative effect on their own. For example, in DB cells, Compound 3 and Compound 1 at the second lowest concentration tested elicited a growth inhibition of only 1 and 2%, respectively, while the respective combination of the two compounds afforded a growth inhibition of 96% (FIG. 4A, left panel), thus being 91% above the additivity calculated based on the single agent activities (FIG. 4A, right panel). As an additional example, in Toledo cells, in which Compound 3 was less potent and achieved only partial growth inhibition (46%) at the highest concentration tested, the combination with the second lowest concentrations of Compound 1 resulted in synergistic growth inhibition of 98% (FIG. 4B, left panel), thus being 52% above the additivity calculated based on the single agent activities (FIG. 4B, right panel).

Furthermore, it is noteworthy that the synergistic effects occurred across a broad range of single agent concentrations, which should prove beneficial in vivo with respect to flexibility concerning dosing levels and scheduling.

In summary, the combination of Compound 3 and Compound 1 afforded strong to exceptional synergistic growth inhibition in the majority of DLBCL cell lines tested.

### EXAMPLE 6: In Vivo EFFICACY IN KARPAS422 XENOGRAFTS WITH COMBINATION OF A MCL1 INHIBITOR (COMPOUND 3) AND A BCL-2 INHIBITOR (COMPOUND 1)

### Material and Method

### Tumour Cell Culture and Cell Inoculation

Karpas 422 human B-cell non-Hodgkin's lymphoma (NHL) cell line was established from the pleural effusion of a patient with chemotherapy-resistant NHL. The cells were obtained from the DSMZ cell bank and cultured in RPMI-1640 medium (BioConcept Ltd. Amimed,) supplemented with 10% FCS (BioConcept Ltd. Amimed), 2 mM L-glutamine (BioConcept Ltd. Amimed), 1 mM sodium pyruvate (BioConcept Ltd. Amimed) and 10 mM HEPES (Gibco) at 37° C. in an atmosphere of 5% CO₂ in air. Cells were maintained between 0.5 and 1.5×106 cells/mL. To establish Karpas 422 xenografts cells were harvested and re-suspended in HBSS (Gibco) and mixed with Matrigel (BD Bioscience) (1:1 v/v) before injecting 200 μL containing 1×107 cells subcutaneously in the right flanks of animals which were anesthetized with isoflurane. Twenty four hours prior to cell inoculation all animals were irradiated with 5Gy over 2 minutes using a γ-irradiator.

### Tumour Growth

Tumour growth was monitored regularly post cell inoculation and animals were randomised into treatment groups (n=5) when tumour volume reached appropriate volume. During the treatment period tumour volume was measured about twice a week using calipers. Tumour size, in mm³, was calculated from: (L×W2×π/6). Where W=width and L=length of the tumour.

### Treatment

Tumour bearing animals (rats) were enrolled into treatment groups (n=5) when their tumours reached an appropriate size to form a group with a mean tumour volume of about 450 mm³. The treatment groups were as outlined in Table 3. The vehicle for Compound 1, HCl or Compound 1, HCl was administered by oral (po) gavage 1 h before vehicle for Compound 3 or Compound 3 which was administered by 15 minutes iv infusion. For iv infusion animals were anesthetized with isoflurane/O₂ and the vehicle or Compound 3 administered via a cannula in the tail vein. Animals were weighed at dosing day(s) and dose was body weight adjusted, dosing volume was 10 ml/kg for both compounds.

### Body Weights

Animals were weighed at least 2 times per week and examined frequently for overt signs of any adverse effects.

### Data Analysis and Statistical Evaluation

Tumour data were analyzed statistically using GraphPad Prism 7.00 (GraphPad Software). If the variances in the data were normally distributed, the data were analyzed using one-way ANOVA with post hoc Dunnett's test for comparison of treatment versus control group. The post hoc Tukey's test was used for intragroup comparison. Otherwise, the Kruskal-Wallis ranked test post hoc Dunn's was used. When applicable, results are presented as mean±SEM.

As a measure of efficacy the % T/C value is calculated at the end of the experiment according to:

(Δtumour volume^{treated}/Δtumour volume^{control})*100

Tumour regression was calculated according to:

−(Δtumour volume^{treated}/tumour volume^{treated at start})*100

wherein Atumour volumes represent the mean tumour volume on the evaluation day minus the mean tumour volume at the start of the experiment.

TABLE 3
Treatment groups for combination efficacy in Karpass422 xenograft bearing rats

| | | Dose (expressed | | Number |
|---|---|---|---|---|
| Groups | Treatment | as the free base) | Schedule | of rats |
| 1 | Vehicle for Com- | 10 ml/kg + 10 ml/kg | QW, po 1 h | 5 |
| | pound 1, HCl | | before + | |
| | (PEG400/EtOH/Phosal | | QW, iv | |
| | 50 PG (30/10/60)), po | | infusion | |
| | 1 h before vehicle for | | | |
| | Compound 3, 15 | | | |
| | minutes iv infusion | | | |
| | 10 ml/kg | | | |
| 2 | Vehicle for Compound | 0 mg/kg + 20 mg/kg | QW, po 1 h | 5 |
| | 1, HCl + | | before + | |
| | Compound 3 | | QW, iv | |
| | | | infusion | |
| 3 | Compound 1, HCl + | 150 mg/kg + 0 mg/kg | QW, po 1 h | 5 |
| | Vehicle | | before + | |
| | for Compound 3 | | QW, iv | |
| | | | infusion | |
| 4 | Compound 1, HCl + | 150 mg/kg + 20 mg/kg | QW, po 1 h | 5 |
| | Compound 3 | | before + | |
| | | | QW, iv | |
| | | | infusion | |

Treatments were initiated when the average tumour volume was about 450 mm³. Compound 1, HCl was formulated in PEG400/EtOH/Phosal 50 PG (30/10/60) and Compound 3 was placed in solution.

QW means once-weekly.

### Results

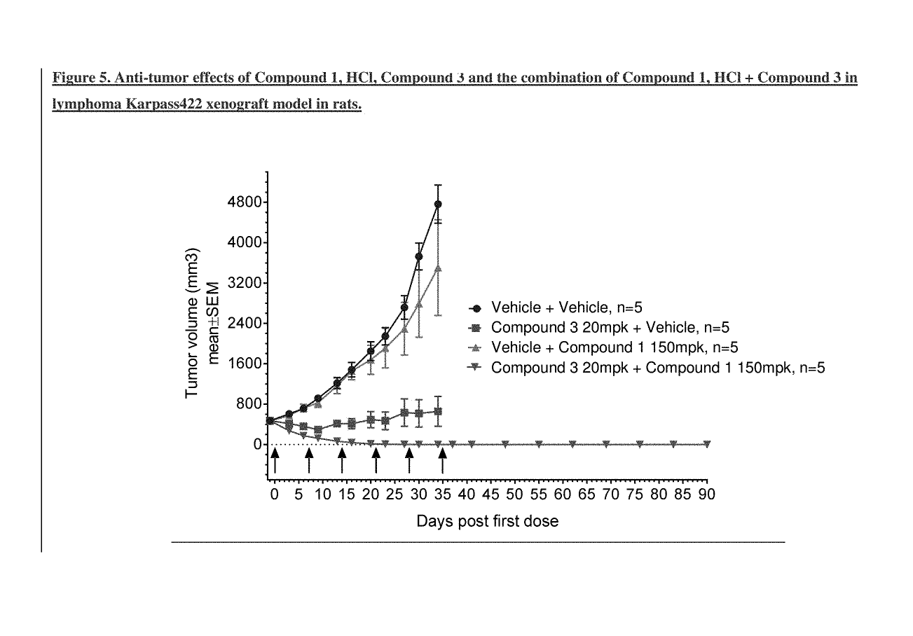

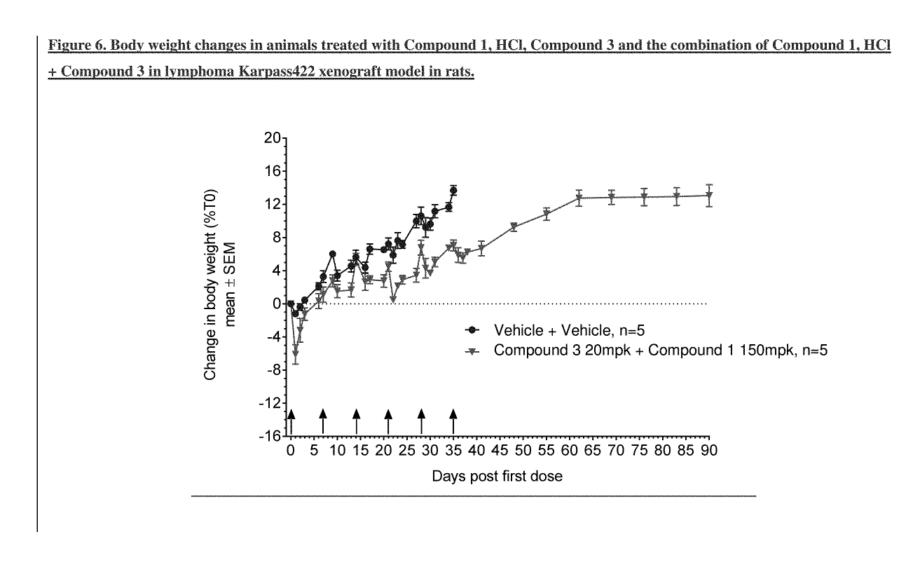

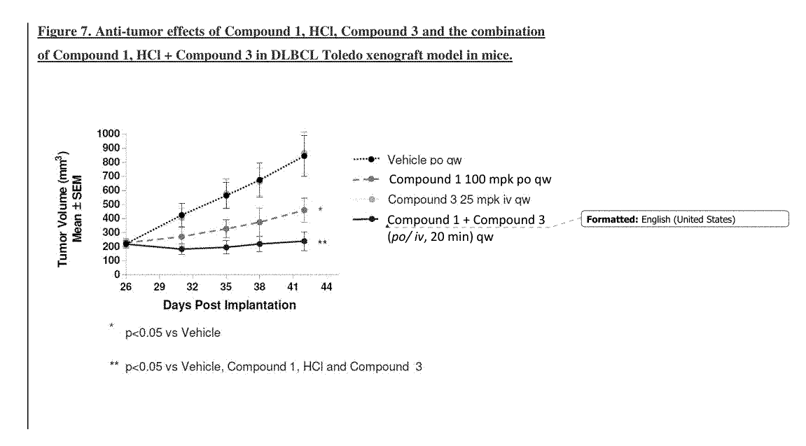

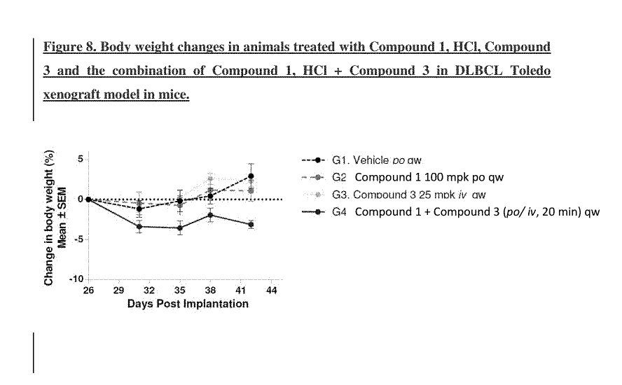

Combination treatment with Compound 1 free base at 150 mg/kg po 1 h before Compound 3 at 20 mg/kg iv infusion induces complete regression in all Karpas422 tumours by day 30 from start of treatment (FIG. 5). All animals in the treatment group have remained tumour free after treatment was stopped on day 35 up to day 90. A positive combination effect is observed in the combination group compared with single agent activity. On day 34 the tumour response in the single agent Compound 3 and the combination group are significantly different from the vehicle group (p<0.05).The combination treatment is well tolerated based on body weight changes (FIG. 6).

### EXAMPLE 7: In VIVO EFFICACY IN DLBCL TOLEDO XENOGRAFT WITH COMBINATION OF A MCL1 INHIBITOR (COMPOUND 3) AND A BCL-2 INHIBITOR (COMPOUND 1, HCL)

### Material and Method

### Cell Implantation

The xenograft model was established by direct subcutaneous (sc) implantation of 3 million Toledo cell suspension with 50% matrigel into the subcutaneous area of SCID/beige mice. All procedures were carried out using aseptic technique. The mice were anesthetized during the entire period of the procedure.

In general, a total of 6 animals per group were enrolled in efficacy study. For single-agent and combination studies, animals were dosed via oral gavage (po) for Compound 1 and intravenously (iv) via tail vein for Compound 3. Compound 1, HCl was formulated as solution in PEG300/EtOH/water (40/10/50), and Compound 3 was placed in solution. When tumors reached approximately 220 mm³ at day 26 post cell implantation, tumour-bearing mice were randomized into treatment groups.

The design of the study including dose schedule for all treatment groups are summarized in the table below. Animals were weighed at dosing day(s) and dose was body weight adjusted, dosing volume was 10 ml/kg. Tumour dimensions and body weights were collected at the time of randomization and twice weekly thereafter for the study duration. The following data was provided after each day of data collection: incidence of mortality, individual and group average body weights, and individual and group average tumour volume.

| | | Dose (expressed | | | Number |
|---|---|---|---|---|---|
| Groups | Treatment | as the free base) | | Schedule | of mice |
| 1 | Vehicle | PEG300/EtOH/water | | QW, po | 6 |
| | | (40/10/50) | | | |
| 2 | Compound 1, | 100 | mg/kg | QW, po | 6 |
| | HCl | | | | |
| 3 | Compound 3 | 25 | mg/kg | QW, iv | 6 |
| 4 | Compound 1, | 100 | mg/kg | QW, po | 6 |
| | HCl + | 25 | mg/kg | QW, iv | |
| | Compound 3 | | | | |

For the study in Toledo model, treatments were initiated on day 26 following cell implantation, when the average tumour volume was ˜218 to 228 mm³.

QW means once-weekly.

### Body Weight (BW)

The % change in body weight was calculated as (BW_{current}−BWᵢₙᵢₜᵢₐₗ)/(BWᵢₙᵢₜᵢₐₗ)×100. Data is presented as percent body weight change from the day of treatment initiation.

### Tumour Volume and Percent Mice Remaining on the Study

Percent treatment/control (T/C) values were calculated using the following formula:

% T/C=100×ΔT/ΔC if ΔT>0

% Regression=100×ΔT/T₀ if ΔT<0

where:
T=mean tumour volume of the drug-treated group on the final day of the study;
ΔT=mean tumour volume of the drug-treated group on the final day of the study—mean tumour volume of the drug-treated group on initial day of dosing;
T₀=mean tumour volume of the drug-treated group on the day of cohort;
C=mean tumour volume of the control group on the final day of the study; and
ΔC=mean tumour volume of the control group on the final day of the study—mean tumour volume of the control group on initial day of dosing.

Percent mice remaining on the study=6−number of mice reaching end point/6*100

### Statistical Analysis

All data were expressed as mean±standard error of the mean (SEM). Delta tumour volume and percent body weight changes were used for statistical analysis. Between group comparisons were carried out using the One way ANOVA followed by a post hoc Tukey test. For all statistical evaluations, the level of significance was set at p<0.05. Significance compared to the vehicle control group is reported unless otherwise stated.

### Results

| Treatment | T/C % at day 42 |
|---|---|
| Vehicle | 100 |
| Compound 1, HCl | 37 |
| Compound 3 | 102 |
| Compound 1, HCl + Compound 3 | 3 |

In Toledo model, Compound 1 free base at 100 mg/kg produced statistically significant anti-tumour effects with 37% T/C. Compound 3 at 25 mg/kg resulted in no anti-tumour effects with 102% T/C (FIG. 7). Combination of Compound 1+Compound 3 led to tumour stasis with 3% T/C, which is statistically significant compared to Vehicle, Compound 1 and Compound 3 treated tumors (p<0.05, by one-way ANOVA test).

Therefore, combined inhibition of BCL-2 and MCL1 in DLBCL may provide a therapeutic benefit in the clinic. In addition, the mean body weight change for Toledo is shown in FIG. 8. Treatment of mice with Compound 1, HCl and Compound 3 exhibit body weight gain (1.081% and 2.3%, respectively). The combination group showed slight body weight loss (−3.2%). No other signs of adverse events were observed in this study. All 6 animals survived throughout the study.

Taken altogether, Examples 2, 6 and 7 show that the combination of a MCL1 inhibitor and a BCL-2 inhibitor is efficacious at tolerated doses in mice and rats bearing xenografts of acute myeloid leukemia and lymphoma human derived cell lines, suggesting that a suitable therapeutic window is achievable with this combination in these diseases.

### EXAMPLE 8: In VITRO EFFECT ON PROLIFERATION OF COMBINING A MCL1 INHIBITOR WITH A BCL-2 INHIBITOR IN A PANEL OF 13 ACUTE MYELOID LEUKEMIA (AML) CELL LINES

### Material and Method

Cell lines were sourced and maintained in the basic media supplemented with FBS (Fetal Bovine Serum) as indicated in Table 1. In addition, all media contained penicillin (100 IU/ml), streptomycin (100 μg/ml) and L-glutamine (2 mM).

Cell lines were cultured at 37° C. in a humidified atmosphere containing 5% CO₂ and expanded in T-150 flasks. In all cases cells were thawed from frozen stocks, expanded through ≥1 passage using appropriate dilutions, counted and assessed for viability using a CASY cell counter prior to plating 150 ul/well at the densities indicated in Table 1 into 96-well plates. All cell lines were determined to be free of mycoplasma contamination in-house.

Stock solutions of compounds were prepared at a concentration of 5 mM in DMSO and stored at −20° C.

In order to analyse the activity of the compounds as single agents, cells were seeded and treated with nine 2-fold serial dilutions of each compound dispensed individually directly into the cell assay plates. Effects of the compounds on cell viability were assessed after 3 days of incubation at 37° C./5% CO₂ by quantification of cellular ATP levels using CellTiterGlo at 75 μL reagent/well. All the experiments were performed in triplicates. Luminescence was quantified on a multipurpose plate reader. Single agent IC₅₀s were calculated using standard four-parametric curve fitting. IC₅₀ is defined as the compound concentration at which the CTG signal is reduced to 50% of that measured for the vehicle (DMSO) control.

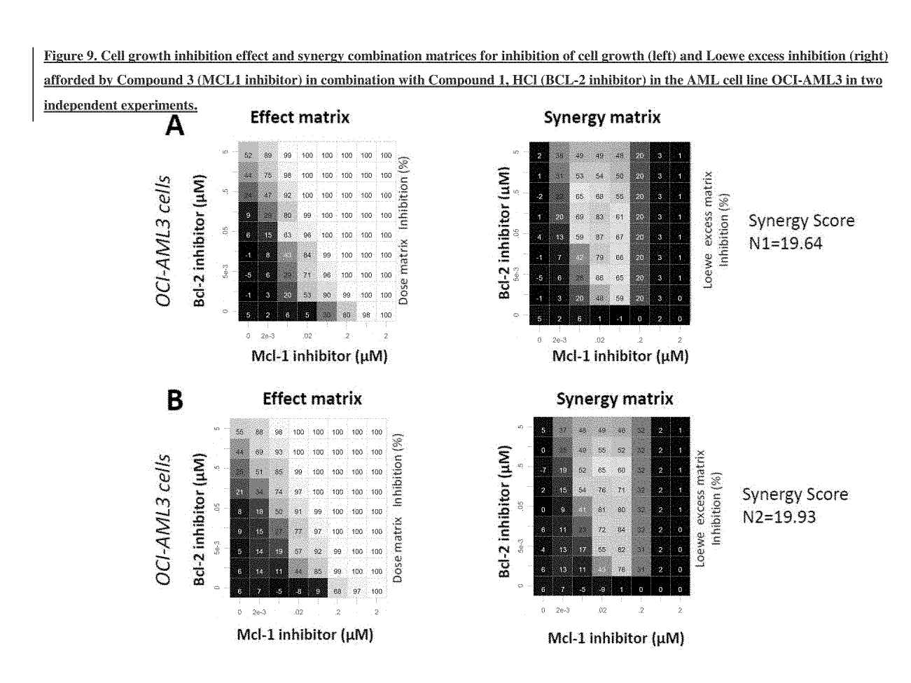

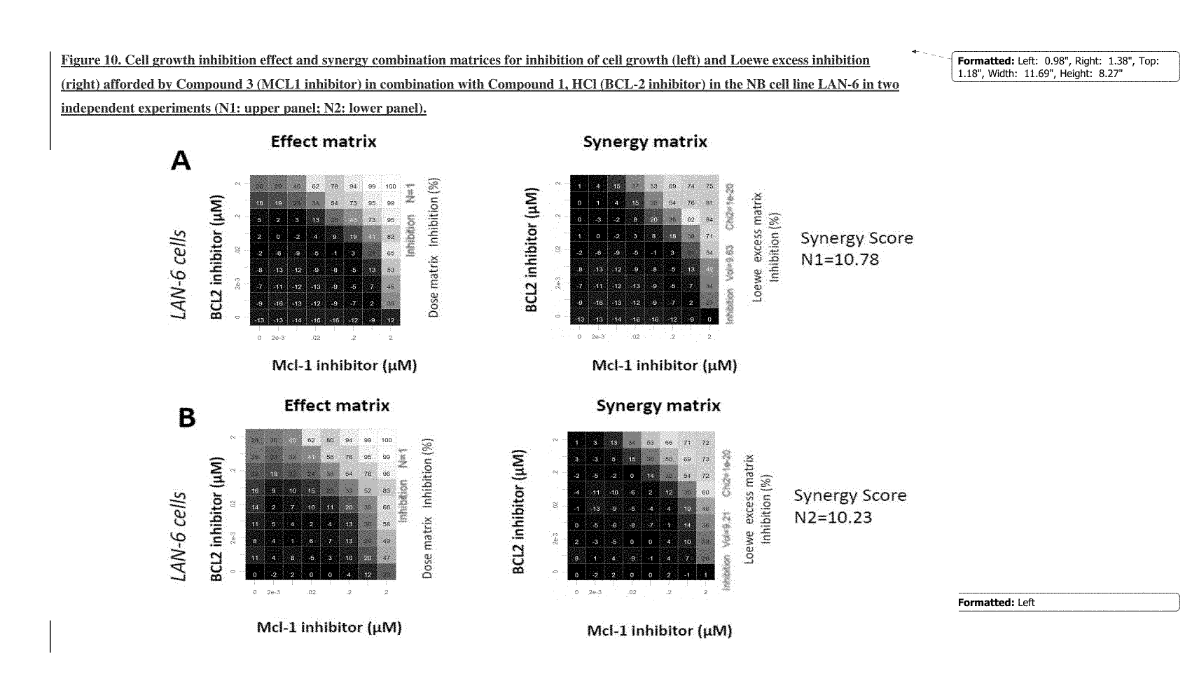

In order to analyse the activity of the compounds in combination, cells were seeded and treated with seven or eight 3.16-fold serial dilutions of each compound dispensed, either individually or in all possible permutations in a checkerboard fashion, directly into the cell assay plates as indicated in FIG. 9. Effects of the single agents as well as their checkerboard combinations on cell viability were assessed after 3 days of incubation at 37° C./5% CO₂ by quantification of cellular ATP levels using CellTiterGlo at 75 μL reagent/well. Two independent experiments, each one performed in duplicates, were performed. Luminescence was quantified on a multipurpose plate reader.

Potential synergistic interactions between compound combinations were assessed using the Excess Inhibition 2D matrix according to the Loewe additivity model and are reported as Synergy Score (Lehar et al, Nat Biotechnol. 2009 July; 27(7): 659-666). All calculations were performed using Clalice™ Bioinformatics Software.

The doubling time indicated in Table 3 is the mean of the doubling time obtained in the different passages (in T-150 flasks) performed from the thawing of the cells to their seeding in the 96-weel plates.

The interpretation of the Synergy Score is as follows:

### SS˜0→Additive

### SS>1→Weak Synergy

### SS>2→Synergy

TABLE 3
Identity and assay conditions for the 13 Acute Myeloid Leukemia
(AML) cell lines used in the combination experiments.

| Cell | Medium | | Doubling | Cell number |
|---|---|---|---|---|
| line | (source) | % FBS | time (hours) | seeded/well |
| MV4;11 | RPMI (ATCC) | 10 | 31.0 | 56520 |
| MOLM-13 | RPMI (DSMZ) | 10 | 32.4 | 56520 |
| PL-21 | RPMI (DSMZ) | 10 | 32.4 | 56520 |
| ML-2 | RPMI (DSMZ) | 10 | 31.6 | 56520 |
| Nomo-1 | RPMI (DSMZ) | 10 | 43.5 | 56520 |
| THP-1 | RPMI (ATCC) | 10 | 49.6 | 56520 |
| HL-60 | IMDM (ATCC) | 20 | 34.8 | 56520 |
| Kasumi-1 | RPMI (ATCC) | 20 | 59.4 | 56520 |
| OCI-AML3 | MEM alpha | 20 | 25.7 | 56520 |
| | (DSMZ) | | | |
| EOL-1 | RPMI (DSMZ) | 10 | 37.6 | 113040 |
| GDM-1 | RPMI (ATCC) | 10 | 31.6 | 56520 |
| KG1 | IMDM (ATCC) | 20 | 45.7 | 56520 |
| KG1a | IMDM (ATCC) | 20 | 36.5 | 56520 |

TABLE 4a
Single agent IC₅₀ values for Compound 3, Compound 1,
HCl and ABT-199 in 13 AML cell lines are indicated. Compounds
were incubated with the cells during 3 days.

| | | | Compound 1, | | | |
|---|---|---|---|---|---|---|
| | Compound 3 | | HCl | | ABT-199 | |
| | Start conc | IC₅₀ | Start conc | IC₅₀ | Start conc | IC₅₀ |
| Cell Line | [uM] | [uM] | [uM] | [uM] | [uM] | [uM] |
| MV4;11 | 0.01 | 0.001 | 0.1 | 0.03 | n.d. | n.d. |
| MOLM-13 | 0.01 | 0.002 | 0.1 | 0.04 | n.d. | n.d. |
| PL-21 | 0.10 | 0.065 | 30.0 | 2.78 | 15.0 | 3.300 |
| ML-2 | 0.10 | 0.005 | 2.0 | 0.04 | n.d. | n.d. |
| Nomo-1 | 0.05 | 0.013 | 30.0 | 7.45 | 15.0 | 5.000 |
| THP-1 | 0.10 | 0.017 | 30.0 | 0.75 | 2.0 | 0.900 |
| HL-60 | 0.10 | 0.025 | 30.0 | 1.42 | 15.0 | 2.100 |
| Kasumi-1 | 2.00 | 0.033 | 30.0 | 0.77 | n.d. | n.d. |
| OCI-AML3 | 2.00 | 0.146 | 30.0 | 8.09 | 15.0 | 8.500 |
| EOL-1 | 0.10 | 0.001 | 2.0 | 0.04 | 0.2 | 0.004 |
| GDM-1 | 0.10 | 0.008 | 2.0 | 0.06 | n.d. | n.d. |
| KG1 | 30.00 | 0.390 | 30.0 | 4.70 | 15.0 | 3.400 |
| KG1a | 30.00 | 2.000 | 30.0 | 1.75 | 15.0 | 0.900 |

TABLE 4b
Single agent IC₅₀ values for Compound 4, HCl
in 5 AML cell lines are indicated. Compound
was incubated with the cells during 3 days.

Compound 4, HCl

| Cell | Start conc | IC₅₀ |
|---|---|---|
| Line | [uM] | [uM] |
| MV4; 11 | 0.5 | 0.01 |
| MOLM-13 | 0.5 | 0.012 |
| ML-2 | 0.5 | 0.01 |
| OCI-AML3 | 15 | 5.41 |
| GDM-1 | 0.5 | 0.002 |

TABLE 5c
Synergy scores for Compound 3 and Compound 4, HCl
combination in 5 AML cell lines are indicated. Interactions
were deemed synergistic when scores ≥ 2.0 where observed.
Start concentrations of compounds, mean of max inhibition
and the standard deviation (sd) of the synergy scores are
indicated. Compounds were incubated with the
cells during 3 days.

| | | | Compound 4, | | | |
|---|---|---|---|---|---|---|
| | Compound 3 | | HCl | | Combination (c) | |
| | | Mean of | | Mean of | | Synergy |
| | Start | Max | Start | Max | | Score |
| | conc | Inh | conc | Inh | Mean of | Error |
| Cell Line | [uM] | [%] | [uM] | [%] | SynergyScore | (sd) |
| MV4;11 | 0.01 | 100.0 | 0.03 | 70 | 3.37 | 0.75 |
| MOLM-13 | 0.1 | 100 | 0.1 | 99 | 3.84 | 0.02 |
| ML-2 | 0.1 | 100 | 0.1 | 99 | 7.09 | 0.96 |
| OCI-AML3 | 2.0 | 100.0 | 5.0 | 53.5 | 16.53 | 1.62 |
| GDM-1 | 0.1 | 100 | 0.1 | 99 | 7.03 | 0.52 |

### Results

Combination (a). The effect on proliferation of combining the MCL1 inhibitor Compound 3 with the BCL-2 inhibitor Compound 1 was assessed in a panel of 13 Acute Myeloid Leukemia (AML) cell lines.

Compound 3 as single agent strongly inhibited the growth of the majority of the 13 AML lines tested (Table 4a). Thus, 10 cell lines displayed IC₅₀s below 100 nM, and an additional 2 cell lines displayed IC₅₀s between 100 nM and 1 uM. Only 1 cell lines displayed an IC₅₀ above 1 uM.

Compound 1, HCl as single agent also inhibited the growth of the several AML lines tested, although slightly less potent (Table 4a). Thus, 5 cell lines displayed IC₅₀s below 100 nM, and 2 cell lines displayed IC₅₀s between 100 nM and 1 uM. Six cell lines displayed an IC₅₀ above 1 uM.

In combination, Compound 3 and Compound 1, HCl treatment caused synergistic growth inhibition (i.e. Synergy Scores above 2) in the entire 13 cell lines tested (Table 5a). In 2 cell lines, the synergy effect was marked, with synergy scores between 5 and 10. In 10 cell lines, the synergy effect was exceptional, achieving synergy scores between 10 and 19.8.

Importantly, the synergy was not dependent on single agent anti-proliferative effects, and in fact was particularly strong at concentrations of Compound 3 and Compound 1 that did not have an anti-proliferative effect on their own. For example, in OCI-AML3 cells, Compound 3 and Compound 1 at the third lowest concentration tested elicited a growth inhibition of 5 and 1%, respectively, while the respective combination of the two compounds afforded a growth inhibition of 84% (FIG. 9A, top left panel), thus being 79% above the additivity calculated based on the single agent activities (FIG. 9A, top right panel).

Furthermore, it is noteworthy that the synergistic effects occurred across a broad range of single agent concentrations, which should prove beneficial in vivo with respect to flexibility concerning dosing levels and scheduling.

In summary, the combination of Compound 3 and Compound 1 afforded synergistic growth inhibition in all the 13 AML cell lines tested. Importantly, exceptional synergistic growth inhibition was observed in the majority AML cell lines tested (10/13).

Combination (b). The effect on proliferation of combining the MCL1 inhibitor Compound 3 with the BCL-2 inhibitor ABT-199 was assessed in a panel of 8 Acute Myeloid Leukemia (AML) cell lines.

Compound 3 as single agent strongly inhibited the growth of the majority of the 8 AML lines tested (Table 4a). Thus, 5 cell lines displayed IC₅₀s below 100 nM, and an additional 2 cell lines displayed IC₅₀s between 100 nM and 1 uM. Only 1 cell lines displayed an IC₅₀ above 1 uM.

ABT-199 as single agent also inhibited the growth of AML lines, although with less potency (Table 4a). Thus, only one cell line displayed IC₅₀s below 100 nM, and 2 cell lines displayed IC₅₀s between 100 nM and 1 uM. Five cell lines displayed IC₅₀ above 1 uM.

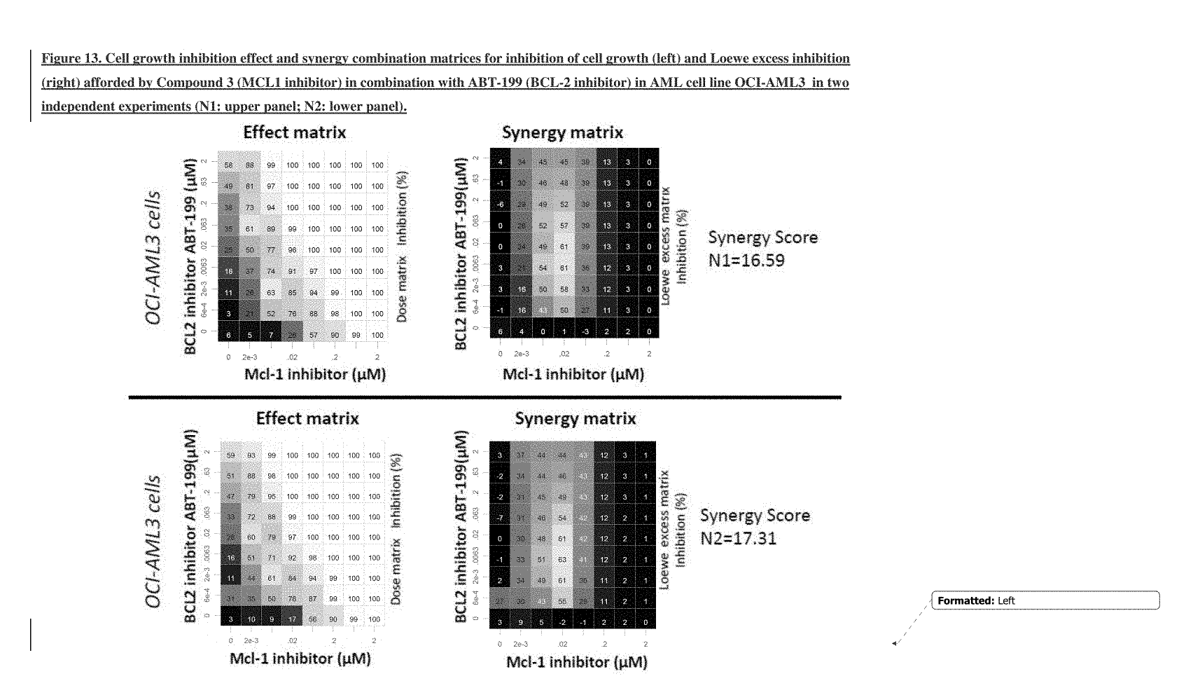

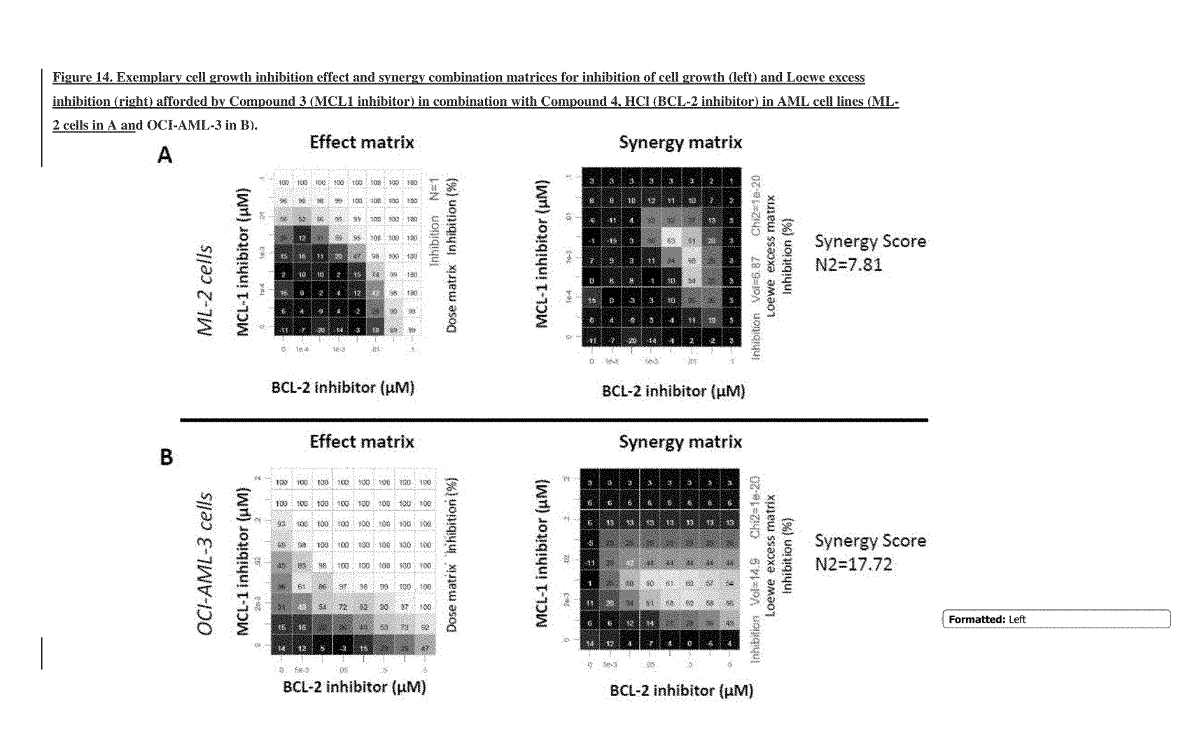

In combination, MCL1 inhibitor and ABT-199 treatment caused synergistic growth inhibition (i.e. Synergy Scores above 2) in the entire panel of 8 cell lines tested (Table 5b). In the majority of the cell lines, the synergy effect was exceptional, achieving synergy scores between 10 and 17.6. Importantly, the synergy was not dependent on single agent anti-proliferative effects, and in fact was particularly strong at concentrations of MCL1 inhibitor and ABT-199 that did not have an anti-proliferative effect on their own. For example, in OCI-AML3 cells, MCL1 and ABT-199 at the third lowest concentration tested elicited a growth inhibition of 26% and 18%, respectively, while the respective combination of the two compounds afforded a growth inhibition of 91% (FIG. 13, top left panel).

Furthermore, it is noteworthy that the synergistic effects occurred across a broad range of single agent concentrations, which should prove beneficial in vivo with respect to flexibility concerning dosing levels and scheduling.

In summary, the combination of Compound 3 and ABT-199 afforded synergistic growth inhibition in all the 8 AML cell lines tested. Importantly, exceptional synergistic growth inhibition was observed in the majority AML cell lines tested (7/8).

Combination (c). The effect on proliferation of combining the MCL1 inhibitor Compound 3 with the BCL-2 inhibitor Compound 4 was assessed in a panel of 5 Acute Myeloid Leukemia (AML) cell lines.

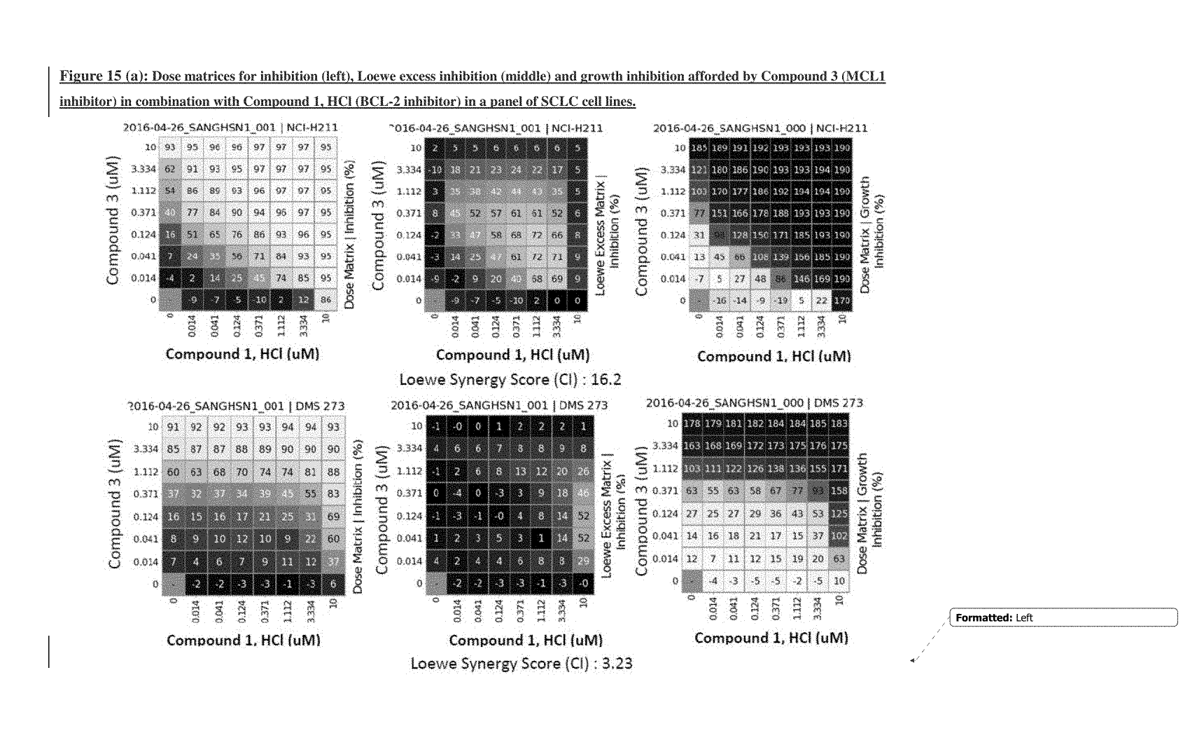

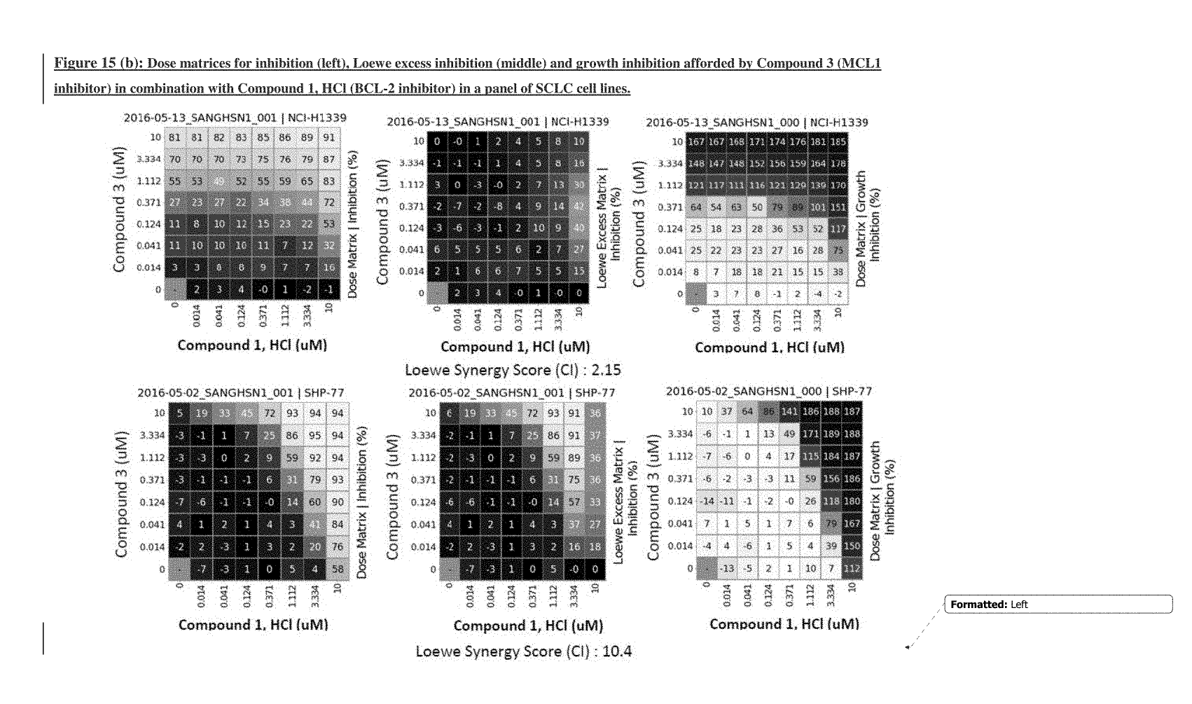

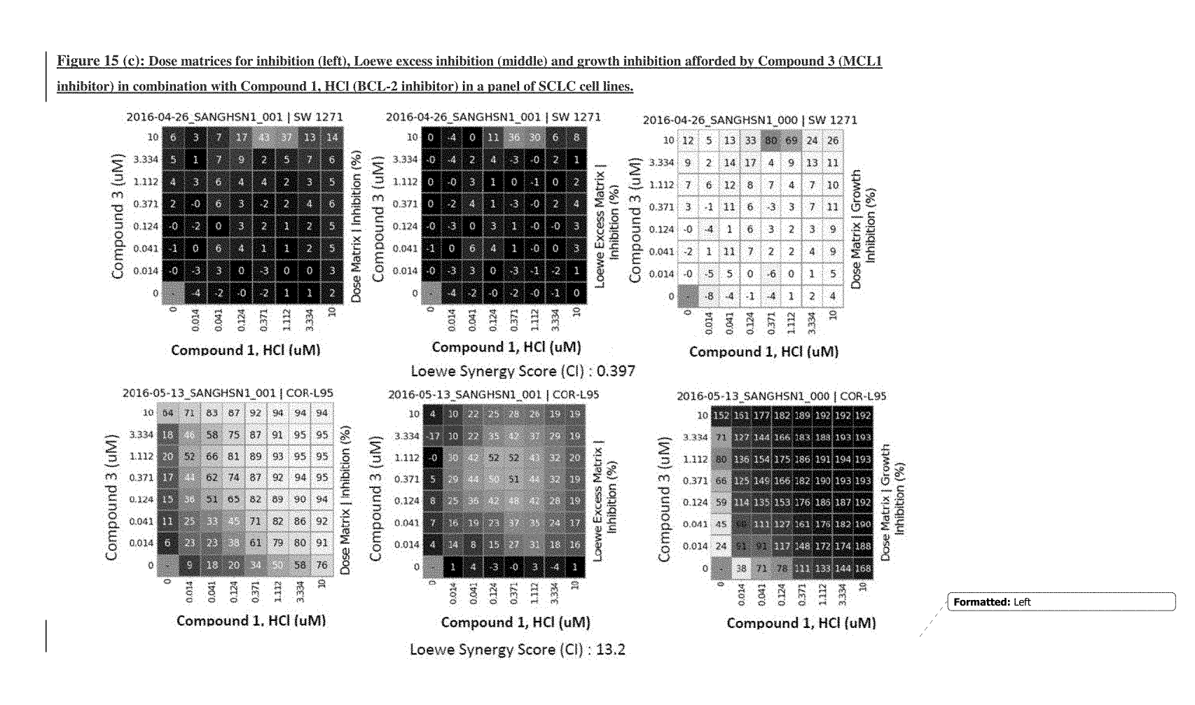

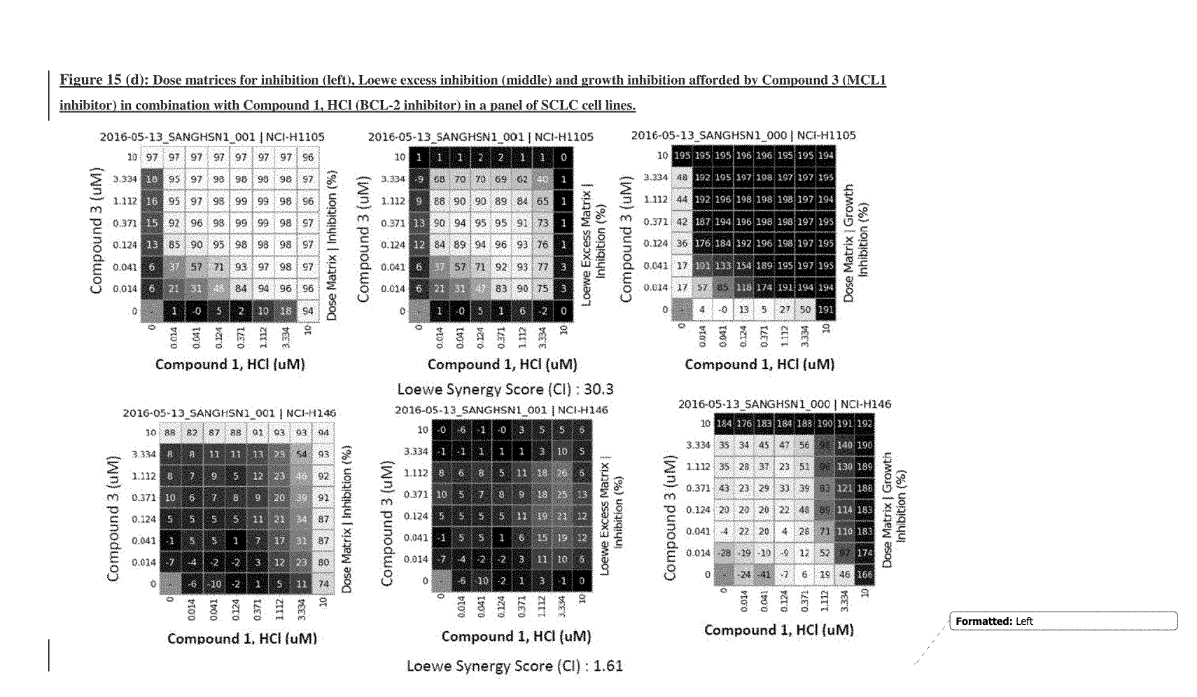

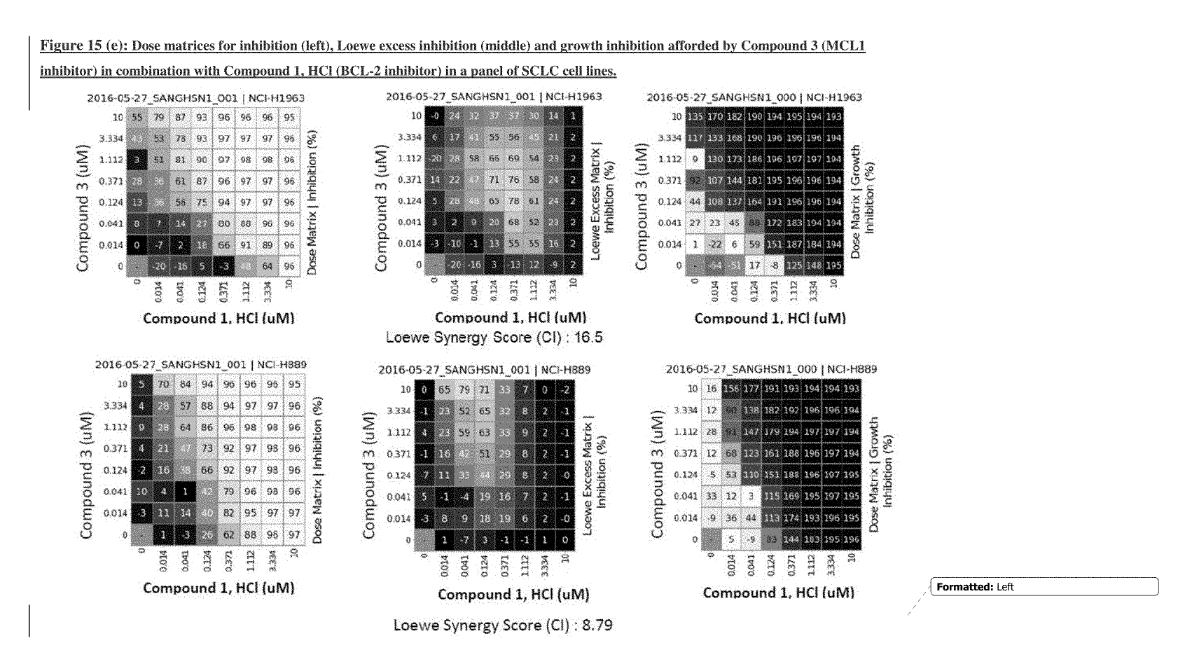

Compound 3 as single agent strongly inhibited the growth of the 5 AML lines tested (Table 4b). Thus, all cell lines displayed IC₅₀s below 200 nM. Compound 4, HCl as single agent also inhibited the growth of the 4 out of 5 cell lines tested with IC₅₀ below or equal to 40 nM, one cell line being resistant to Compound 4 with an IC₅₀ of 10 μM. In combination, Compound 3 and Compound 4, HCl treatment caused synergistic growth inhibition (i.e. Synergy Scores above 2) in the entire 5 cell lines tested (Table 5c). In 2 cell lines, the synergy effect was marked, with synergy scores between 5 and 10. In 1 cell line, the synergy effect was exceptional, achieving synergy score of 16.5. Importantly, the synergy was not dependent on single agent anti-proliferative effects, and in fact was particularly strong at concentrations of Compound 4, HCl and Compound 3 that have no or low anti-proliferative effect on their own. For example, in OCI-AML3 cells, Compound 4, HCl and Compound 3 at the third lowest concentration tested elicited a growth inhibition of 1 and 40%, respectively, while the respective combination of the two compounds afforded a growth inhibition of 98% (FIG. 1A, left panel; representative of two independent experiments), thus being 53% above the additivity calculated based on the single agent activities (FIG. 14A, right panel). In ML-2, Compound 4, HCl and Compound 3 at the fifth lowest concentration tested elicited a growth inhibition of 18 and 26%, respectively, while the respective combination of the two compounds afforded a growth inhibition of 100% (FIG. 14B, left panel; representative of two independent experiments), thus being 51% above the additivity calculated based on the single agent activities (FIG. 15, right panel)

In summary, the combination of Compound 4 and Compound 3 afforded synergistic growth inhibition in all the 5 AML cell lines tested.

### EXAMPLE 9: IN VITRO EFFECT ON PROLIFERATION OF COMBINING A MCL1 INHIBITOR WITH A BCL-2 INHIBITOR IN A PANEL OF OF 12 NEUROBLASTOMA (NB) CELL LINES

### Materials and Methods

Cell lines were sourced and maintained in the basic media supplemented with FBS as indicated in Table 1. In addition, all media contained penicillin (100 IU/ml), streptomycin (100 μg/ml) and L-glutamine (2 mM). Cell lines were cultured at 37° C. in a humidified atmosphere containing 5% CO₂ and expanded in T-150 flasks. In all cases cells were thawed from frozen stocks, expanded through ≥1 passage using appropriate dilutions, counted and assessed for viability using a CASY cell counter prior to plating 150 ul/well at the densities indicated in Table 6 into 96-well plates. All cell lines were determined to be free of mycoplasma contamination in-house.

Stock solutions of compounds were prepared at a concentration of 5 mM in DMSO and stored at −20° C. In order to analyse the activity of the compounds as single agents, cells were seeded and treated with nine 3.16-fold serial dilutions of each compound dispensed individually directly into the cell assay plates. Effects of the compounds on cell viability were assessed after 2 or 3 days of incubation (as indicated in Table 6) at 37° C./5% CO₂ by quantification of cellular ATP levels using CellTiterGlo at 150 μL reagent/well. Two independent experiments, each one performed in duplicates were performed. All the experiments were performed in triplicates. Luminescence was quantified on a multipurpose platereader. Single agent IC₅₀s were calculated using standard four-parametric curve fitting. IC₅₀ is defined as the compound concentration at which the CTG signal is reduced to 50% of that measured for the vehicle (DMSO) control.

Identical experiments were performed to assess potential synergistic interactions between compound combinations. Synergy Score were assessed using the Excess Inhibition 2D matrix according to the Loewe additivity model (Lehar et al, Nat Biotechnol. 2009 July; 27(7): 659-666). All calculations were performed using Chalice TM Bioinformatics Software.

The doubling time indicated in Table 6 is the mean of the doubling time obtained in the different passages (in T-150 flasks) performed from the thawing of the cells to their seeding in the 96-weel plates.

The interpretation of the Synergy Score is as follows:

### SS˜0→Additive

### SS>1→Weak Synergy

### SS>2→Synergy

TABLE 6
Identity and assay conditions for the 12 neuroblastoma
(NB) cell lines used in the combination experiments.

| | | | | | Days of |
|---|---|---|---|---|---|
| Cell | Medium | | Doubling | Cell number | incubation |
| line | (source) | % FBS | time (hours) | seeded/well | with cpds |
| SK-N-AS | DMEM (ATCC) | 10 | 33 | 9375 | 3 |
| SK-N-BE | EMEM/Ham F12 (ATCC) | 10 | 50 | 37500 | 3 |
| SK-N-DZ | DMEM (ATCC) | 10 | 42 | 37500 | 3 |
| LAN-6 | DMEM (DSMZ) | 20 | 100 | 9375 | 3 |
| NBL-S | Iscove's MDM (DSMZ) | 10 | 46 | 18750 | 3 |
| SIMA | RPMI (DSMZ) | 10 | 60 | 18750 | 3 |
| KELLY | RPMI (ECACC) | 10 | 34 | 3750 | 2 |
| IMR-32 | EMEM (ATCC) | 10 | 55 | 28125 | 2 |
| SH-SY-5Y | 1/2 EMEM no glutamin + 1/2 | 15 | 35 | 3750 | 2 |
| | Ham F12 + 2 mM Glutamin + | | | | |
| | NEAA (ECACC) | | | | |
| SK-N-SH | EMEM (ATCC) | 10 | 65 | 3750 | 2 |
| NB-1 | RPMI (JCRB) | 10 | 35 | 15000 | 2 |
| SK-N-FI | DMEM (ATCC) | 10 | 60 | 7500 | 2 |

TABLE 7
Single agent IC₅₀ values for Compound 3 and Compound
1, HCl, are indicated. Compounds were incubated
with the cells during 2 or 3 days.

| Cell | Compound 3 | Compound 1, HCl |
|---|---|---|
| Line | IC₅₀ [uM] | IC₅₀ [uM] |
| SK-N-AS | 0.26 | >1 |
| SK-N-BE | >2 | >2 |
| SK-N-DZ | >2 | >2 |
| LAN-6 | >2 | >2 |
| NBL-S | >2 | >2 |
| SIMA | >2 | >2 |
| NB1 | 0.123 | >3 |
| SK-N-SH | >3 | >3 |
| SH-SY5Y | >3 | >3 |
| Kelly | 0.031 | >3 |
| SK-N-FI | >3 | >3 |

TABLE 8
Synergy scores for combination with Compound 3
and Compound 1, HCl are indicated. Interactions
were deemed synergistic when scores ≥ 2.0 where
observed. Compounds were incubated with the
cells during 2 or 3 days.

| | | | Compound 1, | | | |
|---|---|---|---|---|---|---|
| | Compound 3 | | HCl | | Combination | |
| | | Mean | Start | | | Synergy |
| | Start conc | of Max | conc | Max Inh | Synergy | Score |
| Cell Line | [uM] | Inh [%] | [uM] | [%] | Score | Error |
| SK-N-AS | 2 | 84 | 1 | 9 | 2.78 | 0.46 |
| SK-N-BE | 2 | 27 | 2 | 27 | 10.72 | 0.78 |
| SK-N-DZ | 2 | 2.5 | 2 | 10 | 0.34 | 0.06 |
| LAN-6 | 2 | 17.5 | 2 | 26 | 10.51 | 0.39 |
| NBL-S | 2 | 13 | 2 | 10 | 17.81 | 3.7 |
| SIMA | 2 | 0 | 2 | 48.5 | 2.41 | 0.75 |
| NB1 | 3 | 99 | 3 | 11 | 10.72 | 4.33 |
| SK-N-SH | 3 | 40 | 3 | 15 | 4.07 | 0.23 |
| SH-SY5Y | 3 | 24 | 3 | 10 | 10.21 | 0.54 |
| Kelly | 3 | 99 | 3 | 27 | 9.62 | 0.48 |
| SK-N-FI | 3 | 33 | 3 | 6 | 4.35 | 0.91 |

### Results

The effect on proliferation of combining the MCL1 inhibitor Compound 3 with the BCL-2 inhibitor Compound 1 was assessed in a panel of 12 neuroblastoma cell lines. Three out of the 12 cell lines tested are sensitive to Compound 3 as single agent (Table 7). One cell lines displayed IC₅₀s below 100 nM, and an additional 2 cell lines displayed IC₅₀s between 100 nM and 1 uM.

All cell lines are resistant to Compound 1, HCl as single agent with all cell lines tested displaying an IC₅₀ above 1 μM. In combination, Compound 3 and Compound 1 treatment caused synergistic growth inhibition (i.e. Synergy Scores above 2—Lehar et al, Nat Biotechnol. 2009 July; 27(7): 659-666) in 11 out of 12 NB cell lines tested (Table 8). In 5 cell lines, the synergy effect was exceptional, achieving synergy scores between 10 and 17.81. Importantly, the synergy was not dependent on single agent anti-proliferative effects, and in fact was particularly strong at concentrations of Compound 3 and Compound 1, HCl that did not have an anti-proliferative effect on their own. For example, in LAN-6 cells, Compound 3 and Compound 1, HCl at 630 nM elicited a growth inhibition of only 12% and 0%, respectively, while the respective combination of the two compounds afforded a growth inhibition of 95% (FIG. 10, upper left panel), thus being 76% above the additivity calculated based on the single agent activities (FIG. 10, upper right panel). In summary, the combination of Compound 3 and Compound 1 afforded strong to exceptional synergistic growth inhibition in the majority of neuroblastoma cell lines tested.

### EXAMPLE 10: IN VITRO EFFECT ON PROLIFERATION OF COMBINING A MCL1 INHIBITOR WITH A BCL-2 INHIBITOR IN A PANEL OF 8 B-CELL ACUTE LYMPHOBLASTIC LEUKAEMIA (B-ALL) AND 10 T-CELL ACUTE LYMPHOBLASTIC LEUKAEMIA (T-ALL) CELL LINES

### Materials and Methods

Cell lines were sourced and maintained in the basic media supplemented with FBS as indicated in Table 1. In addition, all media contained penicillin (100 IU/ml), streptomycin (100 μg/ml) and L-glutamine (2 mM). Cell lines were cultured at 37° C. in a humidified atmosphere containing 5% CO₂ and expanded in T-150 flasks. In all cases cells were thawed from frozen stocks, expanded through ≥1 passage using appropriate dilutions, counted and assessed for viability using a CASY cell counter prior to plating 150 ul/well at the densities indicated in Table 9 into 96-well plates. All cell lines were determined to be free of mycoplasma contamination in-house.

Stock solutions of compounds were prepared at a concentration of 5 mM in DMSO and stored at −20° C. In order to analyse the activity of the compounds as single agents, cells were seeded and treated with nine 2-fold serial dilutions of each compound dispensed individually directly into the cell assay plates. Effects of the compounds on cell viability were assessed after 3 days of incubation at 37° C./5% CO₂ by quantification of cellular ATP levels using CellTiterGlo at 75 μL reagent/well. All the conditions were tested in triplicates. Luminescence was quantified on a multipurpose plate reader. Single agent IC₅₀s were calculated using standard four-parametric curve fitting. IC₅₀ is defined as the compound concentration at which the CTG signal is reduced to 50% of that measured for the vehicle (DMSO) control.

In order to analyse the activity of the compounds in combination, cells were seeded and treated with seven or eight 3.16-fold serial dilutions of each compound dispensed, either individually or in all possible permutations in a checkerboard fashion, directly into the cell assay plates as indicated in FIG. 1. Effects of the single agents as well as their checkerboard combinations on cell viability were assessed after 3 days of incubation at 37° C./5% CO₂ by quantification of cellular ATP levels using CellTiterGlo at 75 μL reagent/well. For B-ALL cell lines, two independent experiments, each one performed in duplicates, were performed. For T-ALL cell lines, one experiment performed in triplicate was performed. Luminescence was quantified on a multipurpose plate reader.

Potential synergistic interactions between compound combinations were assessed using the Excess Inhibition 2D matrix according to the Loewe additivity model and are reported as Synergy Score (Lehar et al, Nat Biotechnol. 2009 July; 27(7): 659-666). All calculations were performed using Chalice TM Bioinformatics Software available in Horizon website. The doubling time indicated in Table 9 is the mean of the doubling time obtained in the different passages (in T-150 flasks) performed from the thawing of the cells to their seeding in the 96-well plates.

The interpretation of the Synergy Score is as follows:

### SS˜0→Additive

### SS>1→Weak Synergy

### SS>2→Synergy

TABLE 9
Identity and assay conditions for the 8 B-ALL and 10
T-ALL cell lines used in the combination experiments.

| Cell | Cancer | Medium | | Doubling | Cell number |
|---|---|---|---|---|---|
| line | type | (source) | % FBS | time (hours) | seeded/well |
| TOM-1 | BALL | RPMI (DSMZ) | 20 | 70.0 | 112500 |
| SUP-B15 | BALL | McCoy (DSMZ) | 20 | 35.0 | 112500 |
| NALM-21 | BALL | RPMI (DSMZ) | 10 | 50.0 | 112500 |
| NALM-6 | BALL | RPMI (DSMZ) | 10 | 27.0 | 56250 |
| TANOUE | BALL | RPMI (DSMZ) | 10 | 26.0 | 30000 |
| Kasumi-2 | BALL | RPMI (DSMZ) | 10 | 52.0 | 112500 |
| RS4; 11 | BALL | RPMI (ATCC) | 10 | 42.0 | 90000 |
| BALL-1 | BALL | RPMI (DSMZ) | 10 | 38.0 | 112500 |
| BE-13 | T-ALL | RPMI 1640 (DSMZ) | 10 | 37.0 | 13875 |
| MOLT-4 | T-ALL | RPMI 1640 (ATCC) | 10 | 24.0 | 28125 |
| TALL-104 | T-ALL | IMDM (ATCC) | 20 | 68.0 | 13875 |
| HPB-ALL | T-ALL | RPMI 1640 (DSMZ) | 20 | 42.0 | 56250 |
| DND-41 | T-ALL | RPMI 1640 (DSMZ) | 10 | 38.0 | 56250 |
| CML-T1 | T-ALL | RPMI 1640 (DSMZ) | 10 | 32.0 | 112500 |
| J45.01 | T-ALL | RPMI 1640 (ATCC) | 10 | 25.0 | 56250 |
| CCRF- | T-ALL | RPMI 1640 (ATCC) | 10 | 24.0 | 56250 |
| CEM | | | | | |
| J.RT3 T3.5 | T-ALL | RPMI 1640 (ATCC) | 10 | 24.0 | 56250 |
| Loucy | T-ALL | RPMI 1640 (ATCC) | 10 | 61.0 | 112500 |

TABLE 10
Single agent IC₅₀ values for Compound 3 and Compound 1,
HCl in the 8 B-ALL and 10 T-ALL cell lines are indicated.
Compounds were incubated with the cells during 3 days.

| | | | | | Compound 1, | |
|---|---|---|---|---|---|---|
| | | | Compound 3 | | HCl | |
| | | Treatment | Start | | Start | |
| | Cancer | duration | conc | | conc | |
| Cell Line | type | (h) | [uM] | IC₅₀ [uM] | [uM] | IC₅₀ [uM] |
| TOM-1 | B-ALL | 72 | 0.10 | 0.024 | 0.15 | 0.019 |
| SUP-B15 | B-ALL | 72 | 2.00 | 0.078 | 0.90 | 0.025 |
| NALM-21 | B-ALL | 72 | 0.10 | 0.012 | 0.50 | 0.095 |
| NALM-6 | B-ALL | 72 | 2.00 | 0.120 | 30.00 | 3.630 |
| TANOUE | B-ALL | 72 | 30.00 | 6.540 | 30.00 | 17.000 |
| Kasumi-2 | B-ALL | 72 | 2.00 | 0.030 | 2.00 | 0.209 |
| RS4;11 | B-ALL | 72 | 0.90 | 0.079 | 9.00 | 0.020 |
| BALL-1 | B-ALL | 72 | 0.25 | 0.063 | 0.10 | 0.019 |
| BE-13 | T-ALL | 72 | 0.15 | 0.015 | 30.00 | 6.700 |
| MOLT-4 | T-ALL | 72 | 2.00 | 0.026 | 30.00 | 3.290 |
| TALL-104 | T-ALL | 72 | 2.00 | 0.044 | 30.00 | 15.900 |
| HPB-ALL | T-ALL | 72 | 2.00 | 0.660 | 30.00 | 4.500 |
| DND-41 | T-ALL | 72 | 30.00 | 7.000 | 30.00 | 9.000 |
| CML-T1 | T-ALL | 72 | 30.00 | 6.000 | 30.00 | 15.000 |
| J45.01 | T-ALL | 48 | 0.60 | 0.029 | 30.00 | 9.000 |
| CCRF-CEM | T-ALL | 48 | 0.90 | 0.047 | 30.00 | 7.500 |
| J.RT3 T3.5 | T-ALL | 48 | 1.88 | 0.063 | 30.00 | 10.000 |
| Loucy | T-ALL | 48 | 0.90 | 0.064 | 3.75 | 0.231 |

### Results

The effect on proliferation of combining the MCL1 inhibitor with the BCL-2 inhibitor was assessed in a panel of 8 B-ALL and 10 T-ALL cell lines.

MCL1 inhibitor as single agent strongly inhibited the growth of the majority of the ALL cell lines tested (Table 10). Thus, 13 ALL cell lines displayed IC₅₀s below 100 nM, and an additional 2 ALL cell lines displayed IC₅₀s between 100 nM and 1 uM. Only 3 ALL cell lines displayed IC₅₀ above 1 uM.

BCL-2 inhibitor as single agent also inhibited the growth of several ALL cell lines tested, although it was less potent (Table 10). Thus, 5 cell lines displayed IC₅₀s below 100 nM, and 2 cell lines displayed IC₅₀s between 100 nM and 1 uM. Eleven ALL cell lines displayed an IC₅₀ above 1 uM.

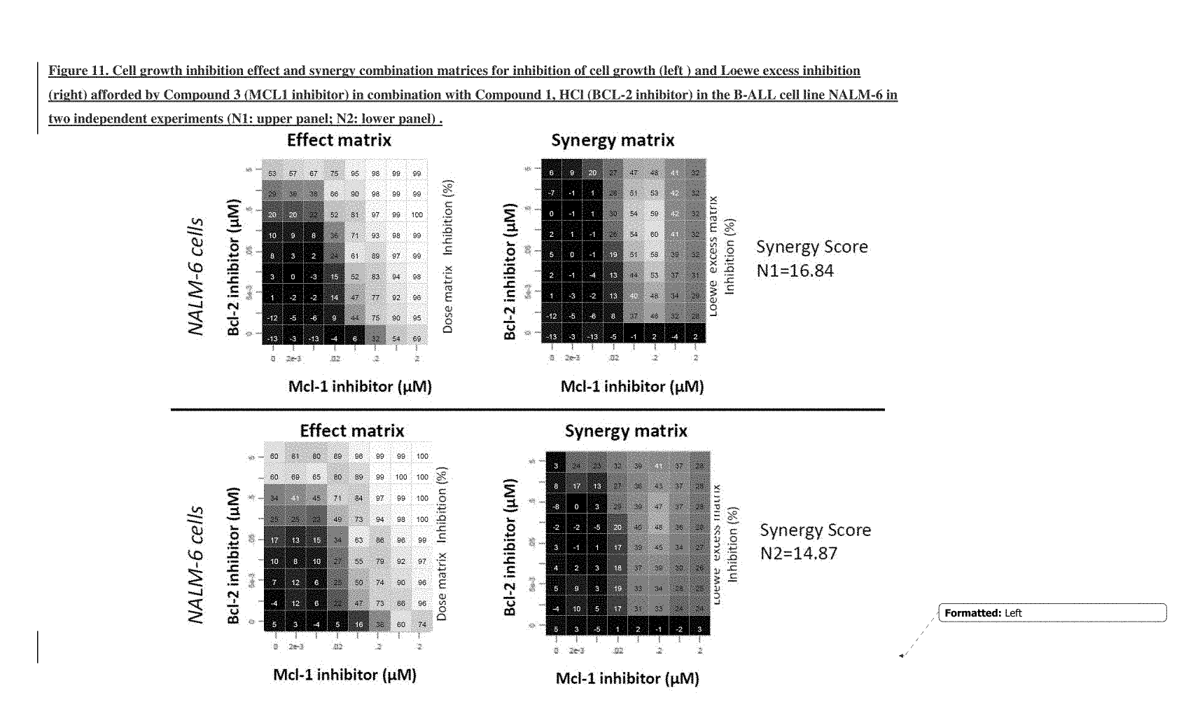

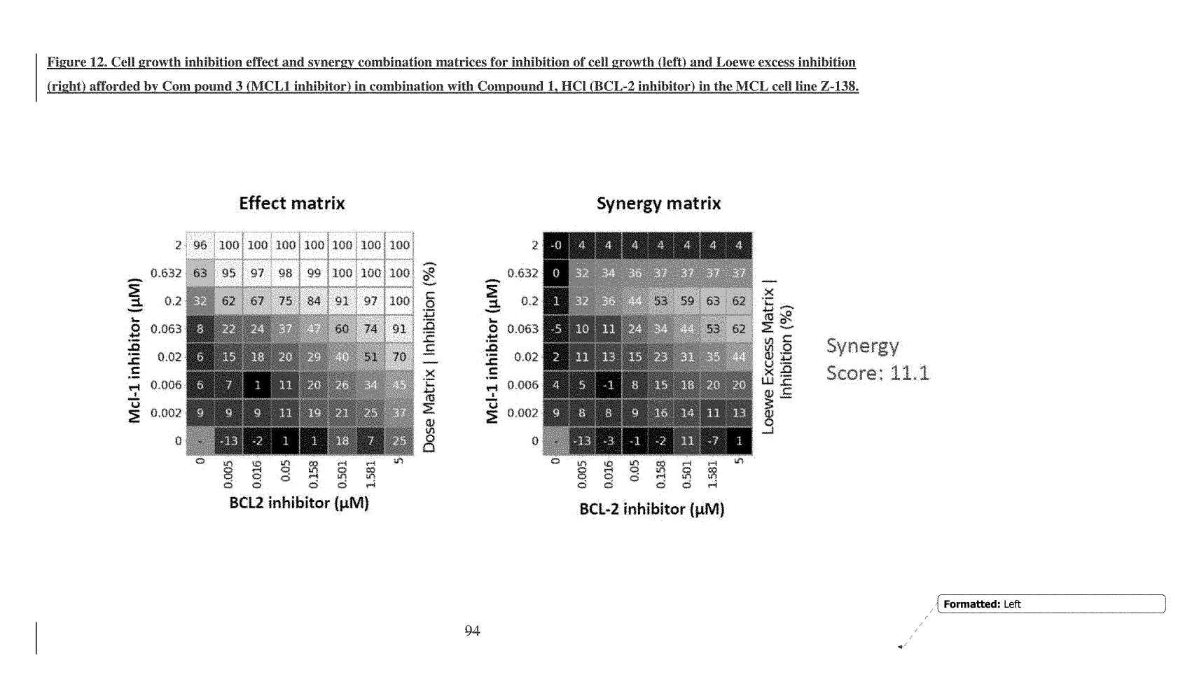

In combination, MCL1 inhibitor and BCL-2 inhibitor treatment caused synergistic growth inhibition (i.e. Synergy Scores above 2—Lehar et al, Nat Biotechnol. 2009 July; 27(7): 659-666) in the entire 17/18 ALL cell lines tested (Table 11). In 6 cell lines, the synergy effect was marked, with synergy scores between 5 and 10. In 5 cell lines, the synergy effect was exceptional, achieving synergy scores between 10 and 15.9. Importantly, the synergy was not dependent on single agent anti-proliferative effects, and in fact was particularly strong at concentrations of MCL1 inhibitor and BCL-2 inhibitor that did not have an anti-proliferative effect on their own. For example, in NALM-6 cells, MCL1 inhibitor and BCL-2 inhibitor at the fourth lowest concentration tested elicited a growth inhibition of 6 and 8%, respectively, while the respective combination of the two compounds afforded a growth inhibition of 61% (FIG. 11, top left panel).

Furthermore, it is noteworthy that the synergistic effects occurred across a broad range of single agent concentrations, which should prove beneficial in vivo with respect to flexibility concerning dosing levels and scheduling.

In summary, the combination of MCL1 inhibitor and BCL-2 inhibitor afforded synergistic growth inhibition in the majority (17/18) of ALL cell lines tested. Importantly, exceptional synergistic growth inhibition was observed in 5/18 ALL cell lines tested.

### EXAMPLE 11: IN VITRO EFFECT ON PROLIFERATION OF COMBINING A MCL1 INHIBITOR WITH A BCL-2 INHIBITOR IN A PANEL OF 5 MANTLE CELL LYMPHOMA (MCL) CELL LINES

### Materials and Methods

Cell lines were sourced and maintained in the basic media supplemented with FBS as indicated in Table 12. In addition, all media contained penicillin (100 IU/ml), streptomycin (100 μg/ml) and L-glutamine (2 mM).

Cell lines were cultured at 37° C. in a humidified atmosphere containing 5% CO₂ and expanded in T-150 flasks. In all cases cells were thawed from frozen stocks, expanded through ≥1 passage using appropriate dilutions, counted and assessed for viability using a CASY cell counter prior to plating 150 ul/well at the densities indicated in Table 12 into 96-well plates. All cell lines were determined to be free of mycoplasma contamination in-house.

Stock solutions of compounds were prepared at a concentration of 5 mM in DMSO and stored at −20° C. In order to analyse the activity of the compounds as single agents or in combination, cells were seeded and treated with seven or eight 3.16-fold serial dilutions of each compound dispensed, either individually or in all possible permutations in a checkerboard fashion, directly into the cell assay plates. Effects of the single agents as well as their checkerboard combinations on cell viability were assessed after 2 days of incubation at 3720 C./5% CO₂ by quantification of cellular ATP levels using CellTiterGlo at 150 μL reagent/well. All the conditions were tested in triplicates. Luminescence was quantified on a multipurpose plate reader.

Potential synergistic interactions between compound combinations were assessed using the Excess Inhibition 2D matrix according to the Loewe additivity model and are reported as Synergy Score (Lehar et al, Nat Biotechnol. 2009 July; 27(7): 659-666). All calculations were performed using Chalice™ Bioinformatics Software available in Horizon website.

Single agent IC₅₀s were calculated using standard four-parametric curve fitting. IC₅₀ is defined as the compound concentration at which the CTG signal is reduced to 50% of that measured for the vehicle (DMSO) control.

The doubling time indicated in Table 12 is the mean of the doubling time obtained in the different passages (in T-150 flasks) performed from the thawing of the cells to their seeding in the 96-weel plates.

### Synergy Score

### SS˜0→Additive

### SS>1→Weak Synergy

### SS>2→Synergy

TABLE 12
Identity and assay conditions for the 5 Mantle Cell Lymphoma
cell lines used in the combination experiments.

| Cell | | | | Doubling | Cell number |
|---|---|---|---|---|---|
| line | Medium | % FBS | Source | time (hours) | seeded/well |
| Z-138 | RPMI | 10 | ATCC | 22.5 | 37500 |
| Jeko | RPMI | 20 | ATCC | 26.0 | 27000 |
| Mino | RPMI | 15 | ATCC | 31.1 | 56250 |
| JVM-2 | RPMI | 10 | ATCC | 76.0 | 56250 |
| REC-1 | RPMI | 10 | ATCC | 36.0 | 56250 |

TABLE 13
Single agent IC₅₀ values for Compound 3 and Compound 1, HCl
in the 5 Mantle Cell Lymphoma cell lines are indicated. Compounds
were incubated with the cells during 2 days.

| | |
|---|---|
| Compound 3 | Compound 1, HCl |

| Cell | Start conc | IC₅₀ | Start conc | IC₅₀ |
|---|---|---|---|---|
| Line | [uM] | [uM] | [uM] | [uM] |
| Z-138 | 2 | 0.448 | 5 | >5 |
| Jeko | 2 | 0.023 | 5 | >5 |
| Mino | 2 | 0.008 | 2 | 0.091 |
| JVM-2 | 2 | >2 | 5 | >5 |
| REC-1 | 2 | 0.077 | 2 | 0.703 |

TABLE 14
Synergy scores for Compound 3 and Compound 1, HCl combination
in 5 Mantle Cell Lymphoma cell lines are indicated. Interactions
were deemed synergistic when scores ≥ 2.0 where observed.
Start concentrations of compounds, max inhibition and
the synergy scores are indicated. Compounds were incubated
with the cells during 2 days.

| | Compound 3 | | Compound 1, HCl | | |
|---|---|---|---|---|---|
| Cell | Start conc | Max Inh | Start conc | Max Inh | Combination |
| Line | [uM] | [%] | [uM] | [%] | Synergy Score |
| Z-138 | 2.0 | 96.0 | 5.0 | 25.0 | 11.1 |
| Jeko | 2.0 | 100.0 | 5.0 | 35.0 | 9.7 |
| Mino | 2.0 | 100.0 | 2.0 | 91.0 | 5.7 |
| JVM-2 | 2.0 | 19.0 | 5.0 | 38.0 | 3.4 |
| REC-1 | 2.0 | 99.0 | 2.0 | 78.0 | 5.1 |

### Results

The effect on proliferation of combining the MCL1 inhibitor with the BCL-2 inhibitor was assessed in a panel of 5 Mantle Cell Lymphoma cell lines.

As single agents, MCL1 inhibitors displayed superior activity as compared with BCL-2 inhibitor. Thus, 3 cell lines displayed IC₅₀s below 100 nM for MCL1 inhibitor while only one cell line displayed IC₅₀s below 100 nM for BCL-2 inhibitor (Table 13).

In combination, MCL1 inhibitor and BCL-2 inhibitor treatment caused synergistic growth inhibition (i.e. Synergy Scores above 2—Lehar et al, Nat Biotechnol. 2009 July; 27(7): 659-666) in all cell lines tested (Table 14), as examplified in FIG. 12. Importantly, in 4/5 cell lines, the synergy effect was marked, with synergy scores above 5.

### EXAMPLE 12: IN VITRO EFFECT ON PROLIFERATION OF COMBINING A MCL1 INHIBITOR WITH A BCL-2 INHIBITOR IN A PANEL OF 5 SMALL CELL LUNG CANCER (SCLC) CELL LINES

All cell lines were obtained from ATCC. Culture media containing RPMI1640 (Invitrogen) supplemented with 10% FBS (HyClone) was used for COR-L95, NCI-H146, NCI-H211, SHP-77, SW1271, NCI-H1339, NCI-H1963, and NCI-H889. Culture media containing Waymouth's MB 752/1 (Invitrogen) with 10% FBS was used for DMS-273. Culture media containing DMEM/F12 (Invitrogen) containing 5% FBS, and supplemented with 0.005 mg/ml insulin, 0.01 mg/ml transferrin, and 30 nM sodium selenite solution (Invitrogen), 10 nM hydrocortisone (Sigma), 10 nM beta-estradiol (Sigma), and 2 mM L-glutamine (HyClone) was used for NCI-H1105.

Cell lines were cultured in 37° C. and 5% CO2 incubator and expanded in T-75 flasks. In all cases cells were thawed from frozen stocks, expanded through >1 passage using 1:3 dilutions, counted and assessed for viability using a ViCell counter (Beckman-Coulter), prior to plating in 384-well. To split and expand cell lines, cells were dislodged from flasks using 0.25% Trypsin-EDTA (GIBCO). All cell lines were determined to be free of mycoplasma contamination as determined by a PCR detection methodology performed at Idexx Radil (Columbia, Mo., USA) and correctly identified by detection of a panel of SNPs.

Cell proliferation was measured in 72hr CellTiter-Glo™ (CTG) assays (Promega G7571) and all results shown are the result of at least triplicate measurements. For CellTiter-Glo™ assays, cells were dispensed into tissue culture treated 384-well plates (Corning 3707) with a final volume of 35 μL of medium and at density of 5000 cells per well. 24 hrs after plating, 5 μL of each compound dilution series were transferred to plates containing the cells, resulting in compound concentration ranges from 0-10 uM and a final DMSO (Sigma D8418) concentration of 0.16%. Plates were incubated for 72 hrs and the effects of compounds on cell proliferation was determined using the CellTiter-Glo™ Luminescent Cell Viability Assay (Promega G7571) and a Envision plate reader (Perkin Elmer).

The CellTiter-Glo® Luminescent Cell Viability Assay is a homogeneous method to determine the number of viable cells in culture based on quantitation of the ATP present, which signals the presence of metabolically active cells. The method is described in detail in the Technical Bulletin, TB288 Promega. Briefly, cells were plated in Opaque-walled multiwell plates in culture medium as described above. Control wells containing medium without cells were also prepared to obtain a value for background luminescence. 15 uL of CellTiter-Glo® Reagent was then added and contents mixed for 10 minutes on an orbital shaker to induce cell lysis. Next, luminescence was recorded using the plate reader.

The percent growth inhibition and excess inhibition were analysed using the Chalice software (CombinatoRx, Cambridge Mass. The percentage of growth inhibition relative to DMSO is displayed in the panel labelled inhibition, and the amount of inhibition in excess of the expected amount in the panel labelled ADD Excess Inhibition (FIGS. 15 (a)-(e)). Concentrations of Compound 1, HCl are shown along the bottom row from left to right and increasing concentrations of Compound 3 along the leftmost column from bottom to top. All remaining points in the grids display results from a combination of the two inhibitors that correspond to the single agent concentrations denoted on the two axes. Data analysis of cell proliferation was performed using Chalice Analyser as described in Lehar et al, Nat Biotechnol. 2009 July; 27(7): 659-666. Excess inhibition was calculated using the Loewe synergy model which measures the effect on growth relative to what would be expected if two drugs behave in a dose additive manner. Positive numbers represent areas of increasing synergy.

### Synergy Score

### SS˜0→Dose Additive

### SS>2→Synergy

### SS>1→Weak Synergy

### Results

In combination, Compound 1 and Compound 3 treatment caused synergistic growth inhibition (i.e. Synergy Scores above 2) in 8/10 small cell lung cancer cell lines. Importantly, in 6 cell lines, the synergy effect was marked, with synergy scores above 6.

### EXAMPLE 13: IN VIVO EFFICACY IN PATIENT-DERIVED PRIMARY AML MODEL HAMLX5343 WITH COMBINATION OF A MCL1 INHIBITOR (COMPOUND 3) AND A BCL-2 INHIBITOR (COMPOUND 1, HCL OR ABT-199)

### Materials and Methods

### Materials

### Animals

NOD scid gamma (NSG) female mice weighing 17-27 grams (Jackson Laboratories) were allowed to acclimate with access to food and water ad libitum for 3 days prior to manipulation.

### Primary Tumor Models

Patient-derived primary AML model HAMLX5343 carrying KRAS mutation and wild type FLT3 were obtained from Dana Farber Cancer Institute.

### Test Compounds, Formulations

Compound 1, HCl was formulated in 5% Ethanol, 20% Dexolve-7 as a solution for intravenous administration or formulated in PEG300/EtOH/water (40/10/50) for oral administration. ABT-199 was formulated in PEG300/EtOH/water (40/10/50) for oral administration. All of them are stable for at least one week at 4° C. Compound 3 was formulated in Liposomal formulation as a solution for intravenous formulation, which is stable for three weeks at 4° C. Vehicle and compound dosing solutions were prepared as needed. All animals were dosed at 10 mL/kg with Compound 1 (expressed as the free base) or ABT-199, or 5 mL/kg with Compound 3.

### Methods

### Study Design

Eight treatment groups were used in study 7844HAMLX5343-XEF as summarized in Table 15. All treatments were initiated when the average tumor burden (% CD-45 positive cells) was between 8% and 15%.

In this study, Compound 1 was administered by oral gavage (po) or intravenous administration at 50 mg/kg once a week, ABT-199 was administered at 25 mg/kg by oral gavage (po) once a week, either as a single agent or in combination with Compound 3 at 12.5 mg/kg once a week, respectively, for 18 days.

Both Compound 1 (expressed as the free base) and ABT-199 were administered at 10 mL/kg. Compound 3 was administered at 5 mL/kg. The dose was body weight adjusted. Bodyweights were recorded twice/week and tumor burden was recorded once/week.

TABLE 15
Doses* and dose schedules for 7844HAMLX5343-XEF

| | Number of | |
|---|---|---|
| Treatment groups | animals | Dosing regimen |
| Vehicle (10 mL/kg) | 4 | QW |
| Compound 1 (50 mg/kg po) | 4 | QW |
| Compound 1 (50 mg/kg iv) | 4 | QW |
| ABT-199 (25 mg/kg po) | 4 | QW |
| Compound 3 (12.5 mg/kg iv) | 4 | QW |
| Compound 1 + Compound 3 (po/iv) | 4 | QW + QW |
| Compound 1 + Compound 3 (iv/iv) | 4 | QW + QW |
| ABT-199 + Compound 3 (po/iv) | 4 | QW + QW |

*Doses are expressed as the free base

### Primary AML Model

For this experiment, 32 mice were implanted with primary AML line HAMLX5343. Mice were injected intravenously with 2.0 million leukemia cells. When the tumor burden was between 8%-15%, animals were randomized into eight groups of four mice each for vehicle, Compound 1 (po), Compound 1 (iv), ABT-199, Compound 3, or combination treatment. After 18 days of treatment, the study was terminated when the tumor burden reached 99%. Tumor burden was measured by FACS analysis.

### Animal Monitoring

Animal well-being and behavior, including grooming and ambulation were monitored twice daily. General health of mice was monitored and mortality recorded daily. Any moribund animals were sacrificed.

### Tumor Measurement

Mice were bled via tail snip once per week. Blood was split into an IgG control well and a CD33/CD45 well of a 96-well plate. Blood was lysed with 200 μl RBC lysis buffer twice at RT, then washed once with FACS buffer (5% FBS in PBS). Samples were then incubated for 10-30 minutes at 4 C in 100 μl blocking buffer (5% mouse Fc Block+5% human Fc Block+90% FACS buffer). 20 μl IgG control mix (2.5 μl Mouse igG1 K isotype control-PE+2.5 μl Mouse igG1 K isotype control-APC+15 μl FACS buffer) were added to the IgG control wells and 20 ul CD33/CD45 mix (2.5 μl Mouse anti-human CD33-PE+2.5 μl Mouse anti-human CD45-APC+15 μl FACS buffer). Samples were incubated for 30-60 minutes at 4 C then washed twice prior to analysis. Samples were run on Canto with FACSDiva software. Analysis was performed with FloJo software. The percent of CD45-positive, live, single cells was reported as the tumor burden.

### Data Analysis

Percent treatment/control (T/C) values were calculated using the following formula:

% T/C=100×ΔT/ΔC if ΔT>0

% Regression=100×ΔT/Tᵢₙᵢₜᵢₐₗ if ΔT<0

where:
T=mean tumor burden of the drug-treated group on the final day of the study;
ΔT=mean tumor burden of the drug-treated group on the final day of the study—mean tumor burden of the drug-treated group on initial day of dosing;
Tᵢₙᵢₜᵢₐₗ=mean tumor burden of the drug-treated group on initial day of dosing;
C=mean tumor burden of the control group on the final day of the study; and
ΔC=mean tumor burden of the control group on the final day of the study—mean tumor burden of the control group on initial day of dosing.

All data were expressed as Mean±SEM. Delta tumor burden and body weight were used for statistical analysis. Between-groups comparisons for final measurements were performed using ANOVA with Tukey's test. Statistical analysis was carried out using GraphPad Prism.

### Statistical Analysis

All data were expressed as mean±standard error of the mean (SEM). Delta tumor volume and body weight were used for statistical analysis. Between-group comparisons were carried out using the Kruskal-Wallis ANOVA followed by a post hoc Dunn's test or Tukey's test. For all statistical evaluations, the level of significance was set at p<0.05. Significance compared to the vehicle control group is reported unless otherwise stated. The standard protocols used in pharmacology studies are not pre-powered to demonstrate statistically significant superiority of a combination over the respective single agent treatment. The statistical power is often limited by potent single agent response and/or model variability. The p-values for combination vs single agent treatments are, however, provided.

### Results

### Synergistic Anti-Tumor Effect of Combined MCL1 and BCL-2 Inhibition

In the 7844HAMLX5343-XEF study, Compound 1, ABT-199 or Compound 3 alone did not show anti-tumor activity in the HAMLX5343 model carrying the KRAS mutation, when administered at 50 mg/kg (oral or iv), 25 mg/kg (oral) or 12.5 mg/kg (iv) once a week, respectively (% T/C of 98, 92, 98 or 99%, respectively, p>0.05).

When orally administered, Compound 1 at 50 mg/kg or ABT-199 at 25 mg/kg in combination with Compound 3 (12.5 mg/kg iv) once a week resulted in tumor stasis (% T/C of 3% or 6%, respectively, p<0.05) in this model.

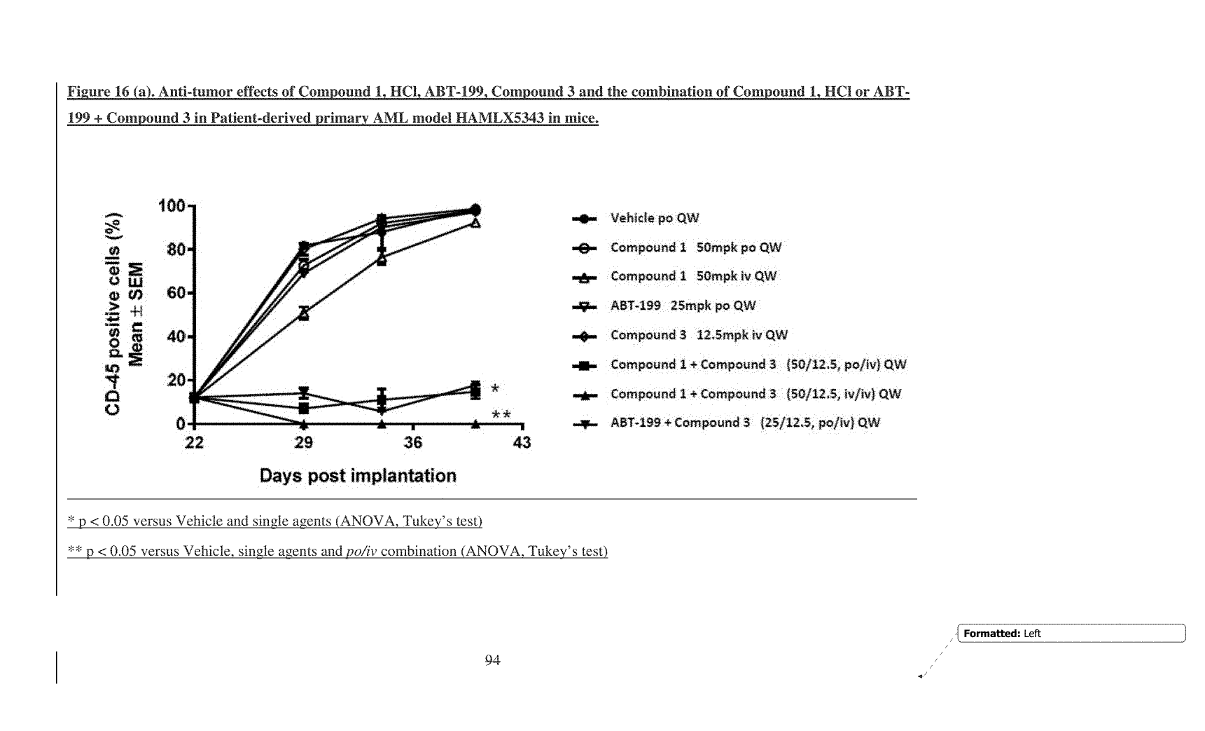

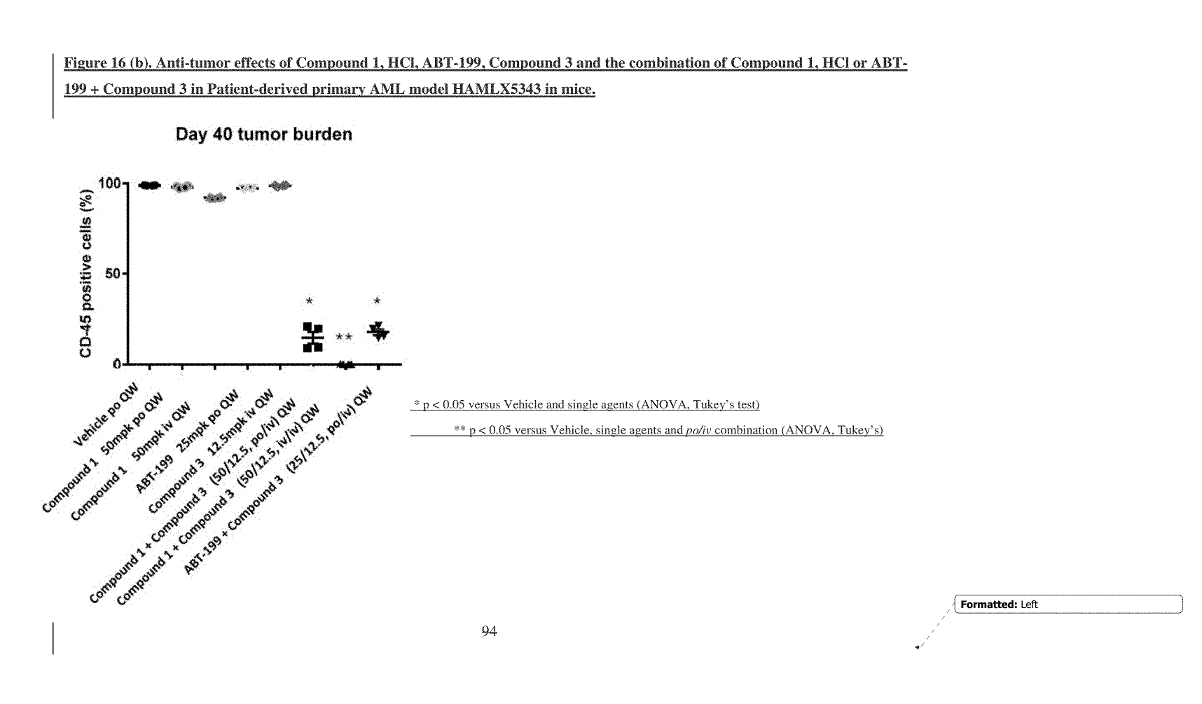

On the other hand, the combination of intravenously administered Compound 1 with Compound 3 induced near complete tumor regression (% Regression of 100%), which is significantly different from either single agent (p<0.05) or Compound 1/Compound 3 po/iv combination. The mean tumor burden for each treatment group is plotted against time for the 18 day treatment period, as shown in FIG. 1. The change in tumor burden, % T/C or % Regression is presented in Table 16 and in FIGS. 16 (a)-(b).

TABLE 16
Summary of anti-tumor effect in 7844HAMLX5343-XEF study

| Treatment | T/C % | Regression % |
|---|---|---|
| Vehicle | 100 | |
| Compound 1 50 mpk po | 98 | |
| Compound 1 50 mpk iv | 92 | |
| ABT-199 25 mpk po | 98 | |
| Compound 3 12.5 mpk iv | 99 | |
| Compound 1 + Compound 3 (po/iv) | 3* | |
| Compound 1 + Compound 3 (iv/iv) | | 100** |
| ABT-199 + Compound 3 (po/iv) | 6* | |

*p < 0.05 versus Vehicle and single agents (ANOVA. Tukey's test)
**p < 0.05 versus poliv combination (ANOVA. Tukey's test)

### CONCLUSION

AML is an aggressive and heterogeneous hematologic malignancy, caused by the transformation of hematopoietic progenitor cells due to acquisition of genetic alterations (Patel et al, New England Journal of Medicine 2012 366:1079-1089). The 5-year survival rate of AML has been low due to lack of effective therapies. Evasion of apoptosis is a hallmark of cancer (Hanahan et al Cell 2000 100:57-70). One of the primary means by which cancer cells evade apoptosis is by up-regulating the pro-survival BCL-2 family proteins such as BCL-2, BCL-xL and MCL1.

MCL1 gene is of the most commonly amplified gene in cancer patients (Beroukhim et al, Nature 2010 463:899-905). Moreover, both BCL-2 and MCL1 are highly expressed in AML. Therefore, the combination of Compound 1 (BCL-2i) and Compound 3 (MCL1) may provide synergy by enhancing pro-apoptotic signals as a general mechanism against AML.

We show here that BCL-2 inhibitor Compound 1 or ABT-199 in combination with Compound 3 (MCL1 inhibitor) has a dramatic synergistic effect in treating AML in an

AML xenograft model with KRAS mutation (wt FLT3). The iv/iv Compound 1/Compound 3 combination is superior to the po/iv combination treatment at the same dose level. The results indicate that the combination of and MCL1 inhibitors would be an effective therapy for AML.

1-46. (canceled)

47. A combination comprising:
(a) a BCL-2 inhibitor of formula (I): 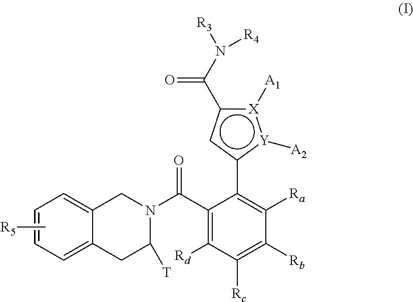
wherein:
X and Y represent a carbon atom or a nitrogen atom, it being understood that they may not simultaneously represent two carbons atoms or two nitrogen atoms,
A₁ and A₂, together with the atoms carrying them, form an optionally substituted, aromatic or non-aromatic heterocycle Het having 5, 6 or 7 ring members which may have, in addition to the nitrogen represented by X or by Y, from 1 to 3 hetero atoms selected independently from oxygen, sulphur and nitrogen, wherein the nitrogen in question may be substituted by a a hydrogen atom, a linear or branched (C₁-C₆)alkyl group or a group —C(O)—O-Alk wherein Alk is a linear or branched (C₁-C₆)alkyl group,
or A₁ and A₂ independently of one another represent a hydrogen atom, a linear or branched (C₁-C₆)polyhaloalkyl, a linear or branched (C₁-C₆)alkyl group or a cycloalkyl,
T represents a hydrogen atom, a linear or branched (C₁-C₆)alkyl group optionally substituted by from one to three halogen atoms, a group (C₁-C₄)alkyl-NR₁R₂, or a group (C₁-C₄)alkyl-OR₆,
R₁ and R₂ independently of one another represent a hydrogen atom or a linear or branched (C₁-C₆)alkyl group,
or R₁ and R₂, together with the nitrogen atom carrying them, form a heterocycloalkyl,
R₃ represents a linear or branched (C₁-C₆)alkyl group, a linear or branched (C₂-C₆)alkenyl group, a linear or branched (C₂-C₆)alkynyl group, a cycloalkyl group, a (C₃-C₁₀)cycloalkyl-(C₁-C₆)alkyl group, wherein the alkyl moiety is linear or branched, a heterocycloalkyl group, an aryl group or a heteroaryl group, wherein one or more of the carbon atoms of the preceding groups, or of their possible substituents, may be deuterated,
R₄ represents an aryl group, a heteroaryl group, a cycloalkyl group or a linear or branched (C₁-C₆)alkyl group, wherein one or more of the carbon atoms of the preceding groups, or of their possible substituents, may be deuterated,
R₅ represents a hydrogen or halogen atom, a linear or branched (C₁-C₆)alkyl group, or a linear or branched (C₁-C₆)alkoxy group,
R₆ represents a hydrogen atom or a linear or branched (C₁-C₆)alkyl group,
Rₐ, R_{b}, R_{c}and R_{d}, each independently of the others, represent R₇, a halogen atom, a linear or branched (C₁-C₆)alkoxy group, a hydroxy group, a linear or branched (C₁-C₆)polyhaloalkyl group, a trifluoromethoxy group, —NR₇R₇′, nitro, R₇—CO—(C₀-C₆)alkyl-, R₇—CO—NH—(C₀-C₆)alkyl-, NR₇R₇′—CO—(C₀-C₆)alkyl-, NR₇R₇′—CO—(C₀-C₆)alkyl-O—, R₇—SO₂—NH—(C₀-C₆)alkyl-, R₇—NH—CO—NH—(C₀-C₆)alkyl-, R₇—O—CO—NH—(C₀-C₆)alkyl-, a heterocycloalkyl group, or the substituents of one of the pairs (Rₐ,R_{b}), (R_{b},R_{c}) or (R_{c},R_{d}), together with the carbon atoms carrying them, form a ring having from 5 to 7 ring members, which may have from 1 to 2 hetero atoms selected from oxygen and sulphur, wherein one or more carbon atoms of the ring defined hereinbefore may be deuterated or substituted by from one to 3 groups selected from halogen and linear or branched (C₁-C₆)alkyl,
R₇ and R₇′ independently of one another represent a hydrogen, a linear or branched (C₁-C₆)alkyl, a linear or branched (C₂-C₆)alkenyl, a linear or branched (C₂-C₆)alkynyl, an aryl or a heteroaryl, or R₇ and R₇′, together with nitrogen atom carrying them, form a heterocycle composed of from 5 to 7 ring members,
wherein when the compound of formula (I) contains a hydroxy group, the latter may be optionally converted into one of the following groups: —OPO(OM)(OM′), —OPO(OM)(O⁻M₁⁺), —OPO(O⁻M₁⁺)(O⁻M₂⁺), —OPO(O)(O⁻)M₃²⁺, —OPO (OM)(O[CH₂CH₂O]ₙCH₃), or —OPO(O⁻M₁⁺)(O[CH₂CH₂O]ₙCH₃), wherein M and M′ independently of one another represent a hydrogen atom, a linear or branched (C₁-C₆)alkyl group, a linear or branched (C₂-C₆)alkenyl group, a linear or branched (C₂-C₆)alkynyl group, a cycloalkyl or a heterocycloalkyl, both having from 5 to 6 ring members, while M₁⁺ and M₂⁺ independently of one another represent a pharmaceutically acceptable monovalent cation, M₃²⁺ represents a pharmaceutically acceptable divalent cation, and n is an integer from 1 to 5,
wherein
“aryl” means a phenyl, naphthyl, biphenyl or indenyl group,
“heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens),
“cycloalkyl” means any mono- or bi-cyclic, non-aromatic, carbocyclic group containing from 3 to 10 ring members,
“heterocycloalkyl” means any mono- or bi-cyclic, non-aromatic, condensed or spiro group composed of 3 to 10 ring members and containing from 1 to 3 hetero atoms selected from oxygen, sulphur, SO, SO₂ and nitrogen,
wherein the aryl, heteroaryl, cycloalkyl and heterocycloalkyl groups so defined and the alkyl, alkenyl, alkynyl and alkoxy groups may be substituted by from 1 to 3 groups selected from the group consisting of: linear or branched (C₁ -C₆)alkyl optionally substituted by a hydroxyl, a morpholine, 3,3-difluoropiperidine or a ,3-difluoropyrrolidine; (C₃-C₆)spiro; linear or branched (C₁-C₆)alkoxy optionally substituted by a morpholine; (C₁-C₆)alkyl-S-; hydroxyl; oxo; N-oxide; nitro; cyano; —COOR′; —OCOR′; NR′R″; linear or branched (C₁-C₆)polyhaloalkyl; trifluoromethoxy; (C₁-C₆)alkylsulphonyl; halogen; aryl optionally substituted by one or more halogens; heteroaryl; aryloxy; arylthio; cycloalkyl; heterocycloalkyl optionally substituted by one or more halogen atoms or alkyl groups, wherein R′ and R″ independently of one another represent a hydrogen atom or a linear or branched (C₁-C₆)alkyl group optionally substituted by a methoxy,
wherein the Het group defined in formula (I) may be substituted by from one to three groups selected from linear or branched (C₁-C₆)alkyl, hydroxy, linear or branched (C₁-C₆)alkoxy, NR₁′R₁″ and halogen, it being understood that R₁′ and R₁′ are as defined for the groups R′ and R″ mentioned hereinbefore,
or its enantiomers, diastereoisomers, or addition salts thereof with a pharmaceutically acceptable acid or base,
and (b) an MCL1 inhibitor,
for simultaneous, sequential or separate use.

48. A combination comprising:
(a) a BCL-2 inhibitor and
(b) an MCL1 inhibitor of formula (II): 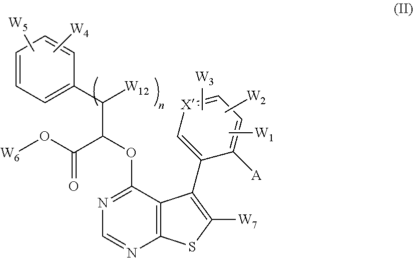
wherein:
A represents a linear or branched (C₁-C₆)alkyl group, a linear or branched (C₂-C₆)alkenyl group, a linear or branched (C₂-C₆)alkynyl group, a linear or branched (C₁-C₆)alkoxy group, —S—(C₁-C₆)alkyl group, a linear or branched (C₁-C₆)polyhaloalkyl, a hydroxy group, a cyano, —NW₁₀W₁₀′, -Cy₆ or a halogen atom,
W₁, W₂, W₃, W₄ and W₅ independently of one another represent a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)polyhaloalkyl, a hydroxy group, a linear or branched (C₁-C₆)alkoxy group, —S—(C₁-C₆)alkyl group, a cyano, a nitro group, -alkyl(C₀-C₆)—NW₈W₈′, —O-Cy₁, -alkyl(C₀-C₆)-Cy₁, -alkenyl(C₂-C₆)-Cy₁, -alkynyl(C₂-C₆)-Cy₁, —O-alkyl(C₁-C₆)—W₉, —C(O)—OW₈, —O—C(O)—W₈, —C(O)—NW₈W₈′, —NW₈—C(O)—W₈′, —NW₈—C(O)—OW₈′, -alkyl(C₁-C₆)—NW₈—C(O)—W₈′, —SO₂— NW₈W₈′, —SO₂-alkyl(C₁-C₆),
or the substituents of one of the pairs (W₁, W₂), (W₂, W₃), (W₁, W₃), (W₄, W₅) when grafted onto two adjacent carbon atoms, together with the carbon atoms carrying them, form an aromatic or non-aromatic ring having from 5 to 7 ring members, which may have from 1 to 3 heteroatoms selected from oxygen, sulphur and nitrogen, wherein the resulting ring may be substituted by a linear or branched (C₁-C₆)alkyl group, —NW₁₀W₁₀′, -alkyl(C₀-C₆)-Cy₁ or an oxo,
X′ represents a carbon or a nitrogen atom,
W₆ represents a hydrogen, a linear or branched (C₁-C₈)alkyl group, an aryl group, a heteroaryl group, an arylalkyl(C₁-C₆) group, or a heteroarylalkyl(C₁-C₆) group,
W₇ represents a linear or branched (C₁-C₆)alkyl group, a linear or branched (C₂-C₆)alkenyl group, a linear or branched (C₂-C₆)alkynyl group, -Cy₃, -alkyl(C₁-C₆)-Cy₃, -alkenyl(C₂-C₆)-Cy₃, -alkynyl(C₂-C₆)-Cy₃, -Cy₃-Cy₄, -alkynyl(C₂-C₆)—O-Cy₃, -Cy₃-alkyl(C₀-C₆)—O-alkyl(Cₒ-C₆)-Cy₄, an halogen atom, a cyano, —C(O)—W₁₁, or —C(O)—NW₁₁W₁₁′,
W₈ and W₈′ independently of one another represent a hydrogen atom, a linear or branched (C₁-C₆)alkyl group, or -alkyl(C₀-C₆)-Cy₁,
or (W₈, W₈′), together with the nitrogen atom carrying them, form an aromatic or non-aromatic ring having from 5 to 7 ring members, which may have, in addition to the nitrogen atom, from 1 to 3 heteroatoms selected from oxygen, sulphur and nitrogen, wherein the nitrogen in question may be substituted by a hydrogen atom, or a linear or branched (C₁-C₆)alkyl group, wherein one or more of the carbon atoms of the possible substituents, may be deuterated,
W₉ represents -Cy₁, -Cy₁-alkyl(C₀-C₆)-Cy₂, -Cy₁-alkyl(C₀-C₆)—O-alkyl(C₀-C₆)-Cy₂, -Cy₁-alkyl(C₀-C₆)—NW₈-alkyl(C₀-C₆)-Cy₂, -Cy₁-Cy₂-O-alkyl(C₀-C₆)-Cy₅, —C(O)—NW₈W₈′, —NW₈W₈′, —OW₈, —NW₈—C(O)—W₈′, —O-alkyl(C₁-C₆)—OW₈, —SO₂—W₈, —C(O)—OW₈, —NH—C(O)—NH—W₈, 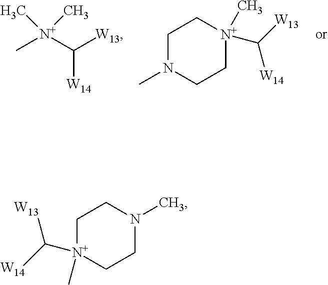
it being possible for the ammonium so defined to exist as a zwitterionic form or to have a monovalent anionic counterion,
W₁₀, W₁₀′, W₁₁ and W₁₁′ independently of one another represent a hydrogen atom or an optionally substituted linear or branched (C₁-C₆)alkyl group,
W₁₂ represents a hydrogen or a hydroxy group,
W₁₃ represents a hydrogen atom or a linear or branched (C₁-C₆)alkyl group,
W₁₄ represents a —O—P(O)(O⁻)(O⁻) group, a —O—P(O)(O⁻)(OW₁₆) group, a —O—P(O)(OW₁₆)(OW_{16′}) group, a —O—SO₂—O⁻ group, a —O—SO₂—OW₁₆ group, -Cy₇, a —O—C(O)—W₁₅ group, a —O—C(O)—OW₁₅ group or a —O—C(O)—NW₁₅W₁₅′ group,
W₁₅ and W₁₅′ independently of one another represent a hydrogen atom, a linear or branched (C₁-C₆)alkyl group or a linear or branched amino(C₁-C₆)alkyl group,
W₁₆ and W₁₆′ independently of one another represent a hydrogen atom, a linear or branched (C₁-C₆)alkyl group or an arylalkyl(C₁-C₆) group,
Cy₁, Cy₂, Cy₃, Cy₄, Cy₅, Cy₆ and Cy₇ independently of one another, represent a cycloalkyl group, a heterocycloalkyl group, an aryl or a heteroaryl group,
n is an integer equal to 0 or 1,
wherein
“aryl” means a phenyl, naphthyl, biphenyl, indanyl or indenyl group,
“heteroaryl” means any mono- or bi-cyclic group having from 5 to 10 ring members, having at least one aromatic moiety and having from 1 to 3 heteroatoms selected from oxygen, sulphur and nitrogen,
“cycloalkyl” means any mono- or bi-cyclic non-aromatic carbocyclic group having from 3 to 10 ring members,
“heterocycloalkyl” means any mono- or bi-cyclic non-aromatic carbocyclic group having from 3 to 10 ring members, and having from 1 to 3 heteroatoms selected from oxygen, sulphur and nitrogen, which may include fused, bridged or spiro ring systems,
wherein the aryl, heteroaryl, cycloalkyl and heterocycloalkyl groups so defined, and the alkyl, alkenyl, alkynyl, alkoxy groups, may be substituted by from 1 to 4 groups selected from the group consisting of linear or branched (C₁-C₆)alkyl which may be substituted by a linear or branched (C₁-C₆)alkoxy which may be substituted by a linear or branched (C₁-C₆)alkoxy, a linear or branched (C₁-C₆)polyhaloalkyl, hydroxy, halogen, oxo, —NW′W″, —O—C(O)—W′, or —CO—NW′W″; a linear or branched (C₂-C₆)alkenyl group; a linear or branched (C₂-C₆)alkynyl group which may be substituted by a linear or branched (C₁-C₆)alkoxy; linear or branched (C₁-C₆)alkoxy which may be substituted by a linear or branched (C₁-C₆)alkoxy, a linear or branched (C₁-C₆)polyhaloalkyl, a linear or branched (C₂-C₆)alkynyl, —NW′W″, or hydroxy; (C₁-C₆)alkyl-S— which may be substituted by a group representing a linear or branched (C₁-C₆)alkoxy; hydroxy; oxo; N-oxide; nitro; cyano; —C(O)—OW′; —O—C(O)—W′; —CO—NW′W″; —NW′W″; —(C═NW′)—OW″; linear or branched (C₁-C₆)polyhaloalkyl; trifluoromethoxy; or halogen, wherein W′ and W″ independently of one another represent a hydrogen atom or a linear or branched (C₁-C₆)alkyl group which may be substituted by a linear or branched (C₁-C₆)alkoxy; and wherein one or more of the carbon atoms of the preceding possible substituents, may be deuterated,
its enantiomers, diastereoisomers and atropisomers, and addition salts thereof with a pharmaceutically acceptable acid or base,
for simultaneous, sequential or separate use.

49. The combination according to claim 47, wherein the MCL1 inhibitor is a compound of formula (II): 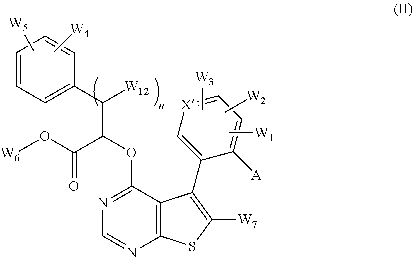
wherein:
A represents a linear or branched (C₁-C₆)alkyl group, a linear or branched (C₂-C₆)alkenyl group, a linear or branched (C₂-C₆)alkynyl group, a linear or branched (C₁-C₆)alkoxy group, —S—(C₁-C₆)alkyl group, a linear or branched (C₁-C₆)polyhaloalkyl, a hydroxy group, a cyano, —NW₁₀W₁₀′, -Cy₆ or a halogen atom,
W₁, W₂, W₃, W₄ and W₅ independently of one another represent a hydrogen atom, a halogen atom, a linear or branched (C₁-C₆)alkyl group, a linear or branched (C₂-C₆)alkenyl group, a linear or branched (C₂-C₆)alkynyl group, a linear or branched (C₁-C₆)polyhaloalkyl, a hydroxy group, a linear or branched (C₁-C₆)alkoxy group, —S—(C₁-C₆)alkyl group, a cyano, a nitro group, -alkyl(C₀-C₆)—NW₈W₈′, —O-Cy₁, -alkyl(C₀-C₆)-Cy₁, -alkenyl(C₂-C₆)-Cy₁, -alkynyl(C₂-C₆)-Cy₁, —O-alkyl(C₁-C₆)—W₉, —C(O)—OW₈, —O—C(O)—W₈, —C(O)—NW₈W₈′, —NW₈—C(O)—W₈′, —NW₈—C(O)—OW₈′, -alkyl(C₁-C₆)—NW₈—C(O)—W₈′, —SO₂— NW₈W₈′, —SO₂-alkyl(C₁-C₆),
or the substituents of one of the pairs (W₁, W₂), (W₂, W₃), (W₁, W₃), (W₄, W₅) when grafted onto two adjacent carbon atoms, together with the carbon atoms carrying them, form an aromatic or non-aromatic ring having from 5 to 7 ring members, which may have from 1 to 3 heteroatoms selected from oxygen, sulphur and nitrogen, wherein the resulting ring may be substituted by a linear or branched (C₁-C₆)alkyl group, —NW₁₀W₁₀′, -alkyl(C₀-C₆)-Cy₁ or an oxo,
X′ represents a carbon or a nitrogen atom,
W₆ represents a hydrogen, a linear or branched (C₁-C₈)alkyl group, an aryl group, a heteroaryl group, an arylalkyl(C₁-C₆) group, or a heteroarylalkyl(C₁-C₆) group,
W₇ represents a linear or branched (C₁-C₆)alkyl group, a linear or branched (C₂-C₆)alkenyl group, a linear or branched (C₂-C₆)alkynyl group, -Cy₃, -alkyl(C₁-C₆)-Cy₃, -alkenyl(C₂-C₆)-Cy₃, -alkynyl(C₂-C₆)-Cy₃, -Cy₃-Cy₄, -alkynyl(C₂-C₆)—O-Cy₃, -Cy₃-alkyl(C₀-C₆)—O-alkyl(C₀-C₆)-Cy₄, an halogen atom, a cyano, —C(O)—W₁₁, or —C(O)—NW₁₁ₗW₁₁′,
W₈ and W₈′ independently of one another represent a hydrogen atom, a linear or branched (C₁-C₆)alkyl group, or -alkyl(C₀-C₆)-Cy₁,
or (W₈, W₈′), together with the nitrogen atom carrying them, form an aromatic or non-aromatic ring having from 5 to 7 ring members, which may have, in addition to the nitrogen atom, from 1 to 3 heteroatoms selected from oxygen, sulphur and nitrogen, wherein the nitrogen in question may be substituted by a hydrogen atom, or a linear or branched (C₁-C₆)alkyl group, wherein one or more of the carbon atoms of the possible substituents, may be deuterated,
W₉ represents -Cy₁, -Cy₁-alkyl(C₀-C₆)-Cy₂, -Cy₁-alkyl(C₀-C₆)—O-alkyl(C₀-C₆)-Cy₂, -Cy₁-alkyl(C₀-C₆)—NW₈-alkyl(C₀-C₆)-Cy₂, -Cy₁-Cy₂-O-alkyl(C₀-C₆)-Cy₅, —C(O)—NW₈W₈′, —NW₈W₈′, —OW₈, —NW₈—C(O)—W₈′, —O-alkyl(C₁-C₆)—OW₈, —SO₂—W₈, —C(O)—OW₈, —NH—C(O)—NH—W₈, 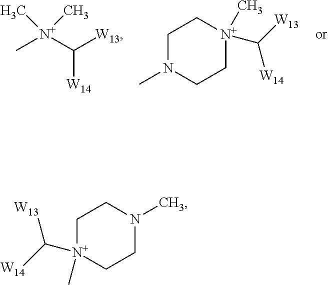
it being possible for the ammonium so defined to exist as a zwitterionic form or to have a monovalent anionic counterion,
W₁₀, W₁₀′, W₁₁ and W₁₁′ independently of one another represent a hydrogen atom or an optionally substituted linear or branched (C₁-C₆)alkyl group,
W₁₂ represents a hydrogen or a hydroxy group,
W₁₃ represents a hydrogen atom or a linear or branched (C₁-C₆)alkyl group,
W₁₄ represents a —O—P(O)(O⁻)(O⁻) group, a —O—P(O)(O⁻)(OW₁₆) group, a —O—P(O)(OW₁₆)(OW₁₆′) group, a —O—SO₂—O⁻ group, a —O—SO₂—OW₁₆ group, -Cy₇, a —O—C(O)—W₁₅ group, a —O—C(O)—OW₁₅ group or a —O—C(O)—NW₁₅W_{15′} group,
W₁₅ and W₁₅′ independently of one another represent a hydrogen atom, a linear or branched (C₁-C₆)alkyl group or a linear or branched amino(C₁-C₆)alkyl group,
W₁₆ and W₁₆′ independently of one another represent a hydrogen atom, a linear or branched (C₁-C₆)alkyl group or an arylalkyl(C₁-C₆) group,
Cy₁, Cy₂, Cy₃, Cy₄, Cy₅, Cy₆ and Cy₇ independently of one another, represent a cycloalkyl group, a heterocycloalkyl group, an aryl or a heteroaryl group,
n is an integer equal to 0 or 1,
wherein
“aryl” means a phenyl, naphthyl, biphenyl, indanyl or indenyl group,
“heteroaryl” means any mono- or bi-cyclic group having from 5 to 10 ring members, having at least one aromatic moiety and having from 1 to 3 heteroatoms selected from oxygen, sulphur and nitrogen,
“cycloalkyl” means any mono- or bi-cyclic non-aromatic carbocyclic group having from 3 to 10 ring members,
“heterocycloalkyl” means any mono- or bi-cyclic non-aromatic carbocyclic group having from 3 to 10 ring members, and having from 1 to 3 heteroatoms selected from oxygen, sulphur and nitrogen, which may include fused, bridged or spiro ring systems,
wherein the aryl, heteroaryl, cycloalkyl and heterocycloalkyl groups so defined, and the alkyl, alkenyl, alkynyl, alkoxy groups, may be substituted by from 1 to 4 groups selected from the group consisting of linear or branched (C₁-C₆)alkyl which may be substituted by a linear or branched (C₁-C₆)alkoxy which may be substituted by a linear or branched (C₁-C₆)alkoxy, a linear or branched (C₁-C₆)polyhaloalkyl, hydroxy, halogen, oxo, —NW′W″, —O—C(O)—W′, or —CO—NW′W″; a linear or branched (C₂-C₆)alkenyl group; a linear or branched (C₂-C₆)alkynyl group which may be substituted by a linear or branched (C₁-C₆)alkoxy; linear or branched (C₁-C₆)alkoxy which may be substituted by a linear or branched (C₁-C₆)alkoxy, a linear or branched (C₁-C₆)polyhaloalkyl, a linear or branched (C₂-C₆)alkynyl, —NW′W″, or hydroxy; (C₁-C₆)alkyl-S— which may be substituted by a group representing a linear or branched (C₁-C₆)alkoxy; hydroxy; oxo; N-oxide; nitro; cyano; —C(O)—OW′; —O—C(O)—W′; —CO—NW′W″; —NW′W″; —(C═NW′)—OW″; linear or branched (C₁-C₆)polyhaloalkyl; trifluoromethoxy; or halogen, wherein W′ and W″ independently of one another represent a hydrogen atom or a linear or branched (C₁-C₆)alkyl group which may be substituted by a linear or branched (C₁-C₆)alkoxy; and wherein one or more of the carbon atoms of the preceding possible substituents, may be deuterated,
its enantiomers, diastereoisomers and atropisomers, and addition salts thereof with a pharmaceutically acceptable acid or base.

50. The combination according to claim 47, wherein the BCL-2 inhibitor is N-(4-hydroxyphenyl)-3-{6-[((3S)-3-(4-morpholinylmethyl)-3,4-dihydro-2(1H)-isoquinolinyl) carbonyl]-1,3-benzodioxol-5-yl}-N-phenyl-5,6,7,8-tetrahydro-1-indolizine carboxamide.

51. The combination according to claim 47, wherein the BCL-2 inhibitor is 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide.

52. The combination according to claim 50, wherein N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide is in the form of the hydrochloride salt.

53. The combination according to claim 51, wherein 5-(5-chloro-2-{[(3S)-3-(morpholin-4-ylmethyl)-3,4-dihydroisoquinolin-2(1H)-yl]carbonyl}phenyl)-N-(5-cyano-1,2-dimethyl-1H-pyrrol-3 -yl)-N-(4-hydroxyphenyl)-1,2-dimethyl-1H-pyrrole-3 -carboxamide is in the form of the hydrochloride salt.

54. The combination according to claim 50, wherein the dose of N-(4-hydroxyphenyl)-3-{6-[((3S)-3-(4-morpholinylmethyl)-3,4-dihydro-2(1H)-isoquinolinyl)carbonyl]-1,3-benzodioxol -5-yl}-N-phenyl-5,6,7,8-tetrahydro-1-indolizine carboxamide is from 50 mg to 1500 mg.

55. The combination according to claim 47, wherein the BCL-2 inhibitor is administered once a week.

56. The combination according to claim 52, wherein N-(4-hydroxyphenyl)-3-{6-[((3S)-3-(4-morpholinylmethyl)-3,4-dihydro-2(1H)-isoquinolinyl)carbonyl]-1,3-benzodioxol-5-yl}-N-phenyl-5,6,7,8-tetrahydro-1-indolizine carboxamide is administered during the combination treatment once a day.

57. The combination according to claim 47, wherein the BCL-2 inhibitor is ABT-199.

58. The combination according to claim 47, wherein the MCL1 inhibitor is (2R)-2-{[(5Sₐ)-5-{3-chloro-2-methyl-4-[2-(4-methylpiperazin-1-yl)ethoxy]phenyl}-6-(5-fluorofuran-2-yl)thieno[2,3-d]pyrimidin-4-yl]oxy}-3-(2-{[1-(2,2,2-trifluoroethyl)-1H-pyrazol-5-yl]methoxy}phenyl)propanoic acid.

59. The combination according to claim 47, wherein the MCL1 inhibitor is (2R)-2-{[(5Sₐ)-5-{3-chloro-2-methyl-4-[2-(4-methylpiperazin-1-yl)ethoxy]phenyl}-6-(4-fluorophenyl)thieno[2,3-d]pyrimidin-4-yl]oxy}-3-(2-{[2-(2-methoxyphenyl)pyrimidin-4-yl]methoxy}phenyl)propanoic acid.

60. The combination according to claim 47, wherein the BCL-2 inhibitor and the MCL1 inhibitor are administered orally.

61. The combination according to claim 47, wherein the BCL-2 inhibitor is administered orally and the MCL1 inhibitor is administered intravenously.

62. The combination according to claim 47, wherein the BCL-2 inhibitor and the MCL1 inhibitor are administered intravenously.

63. A method of treating cancer in a subject in need thereof, comprising administration of an effective amount of the combination according to claim 47, alone or in combination with one or more pharmaceutically acceptable excipients.

64. The method according to claim 63, wherein the BCL-2 inhibitor and the MCL1 inhibitor are provided in amounts which are jointly therapeutically effective for the treatment of cancer.

65. The method according to claim 63, wherein the BCL-2 inhibitor and the MCL1 inhibitor are provided in amounts which are synergistically effective for the treatment of cancer.

66. The method according to claim 63, wherein the BCL-2 inhibitor and the MCL1 inhibitor are provided in synergistically effective amounts which enable a reduction of the dose required for each compound in the treatment of cancer, whilst providing an efficacious cancer treatment, with eventually a reduction in side effects.

67. The method according to claim 63, wherein the cancer is leukaemia.

68. The method according to claim 67, wherein the leukaemia is acute myeloid leukaemia, T-ALL or B-ALL.

69. The method according to claim 63, wherein the cancer is myelodysplastic syndrome or myeloproliferative disease.

70. The method according to claim 63, wherein the cancer is lymphoma.

71. The method according to claim 70, wherein the lymphoma is a non-Hodgkin lymphoma.

72. The method according to claim 71, wherein the non-Hodgkin lymphoma is diffuse large B-cell lymphoma or mantle-cell lymphoma.

73. The method according to claim 63, wherein the cancer is multiple myeloma.

74. The method according to claim 63, wherein the cancer is neuroblastoma.

75. The method according to claim 63, wherein the cancer is small cell lung cancer.

76. A composition comprising the combination according to claim 47 and one or more excipients.

77. A pharmaceutical composition containing, separately or together,
(a) the BCL-2 inhibitor of formula (I) as defined in claim 47, and
(b) an MCL1 inhibitor,
for simultaneous, sequential or separate administration, wherein the BCL-2 inhibitor and the MCL1 inhibitor are provided in effective amounts for the treatment of cancer.

78. A pharmaceutical composition containing, separately or together,
(a) a BCL-2 inhibitor, and
(b) the MCL1 inhibitor of formula (II) as defined in claim 48,
for simultaneous, sequential or separate administration, and wherein the BCL-2 inhibitor and the MCL1 inhibitor are provided in effective amounts for the treatment of cancer.

79. A method of treating cancer, comprising administering a jointly therapeutically effective amount of (a) the BCL-2 inhibitor of formula (I) as defined in claim 47, and
(b) an MCL1 inhibitor,
to a subject in need thereof.

80. A method of treating cancer, comprising administering a jointly therapeutically effective amount of (a) a BCL-2 inhibitor, and
(b) the MCL1 inhibitor of formula (II) as defined in claim 48,
to a subject in need thereof.

81. A method for sensitizing a patient who is (i) refractory to at least one chemotherapy treatment, or (ii) in relapse after treatment with chemotherapy, or both (i) and (ii), wherein the method comprises administering a jointly therapeutically effective amount of (a) the BCL-2 inhibitor of formula (I) as defined in claim 47, and (b) an MCL1 inhibitor, to the patient.

82. A method for sensitizing a patient who is (i) refractory to at least one chemotherapy treatment, or (ii) in relapse after treatment with chemotherapy, or both (i) and (ii), wherein the method comprises administering a jointly therapeutically effective amount of (a) a BCL-2 inhibitor, and (b) the MCL1 inhibitor of formula (II) as defined in claim 48, to the patient.
